(19) United States
(12) Patent Application Publication
(10) Pub. No.: US 2024/0366587 A1
(43) Pub. Date: Nov. 7, 2024

(54) USES OF A SOMATOSTATIN MODULATOR FOR THE TREATMENT OF DISEASE

(71) Applicant: Crinetics Pharmaceuticals, Inc., San Diego, CA (US)

(72) Inventors: Ajay MADAN, San Diego, CA (US); Sha Rosa LUO, San Diego, CA (US); Alan S. KRASNER, San Diego, CA (US)

(21) Appl. No.: 18/762,266

(22) Filed: Jul. 2, 2024

Related U.S. Application Data
(63) Continuation of application No. 17/752,526, filed on May 24, 2022.
(60) Provisional application No. 63/193,010, filed on May 25, 2021.
(60) Provisional application No. 63/274,409, filed on Nov. 1, 2021.

(51) Int. Cl. A61K 31/4709; A61K 9/00; A61P 5/06

(52) U.S. Cl. CPC A61K 31/4709; A61K 9/0053; A61P 5/06

(57) ABSTRACT

Described herein are formulations of a somatostatin modulator, methods of making such formulations, and methods of using such formulations in the treatment of conditions, diseases, or disorders that would benefit from modulation of somatostatin activity.

### CROSS-REFERENCE TO RELATED APPLICATIONS

This application is a continuation of U.S. patent application Ser. No. 17/752,526, filed on May 24, 2022, which claims benefit of U.S. Provisional Patent Application No. 63/193,010, filed on May 25, 2021; and U.S. Provisional Patent Application No. 63/274,409, filed on Nov. 1, 2021; each of which is incorporated herein by reference in its entirety.

### FIELD OF THE INVENTION

Described herein are pharmaceutical compositions and medicaments comprising a somatostatin modulator, methods of making such pharmaceutical compositions and medicaments and methods of using such pharmaceutical compositions and medicaments in the treatment of conditions, diseases, or disorders that would benefit from modulating somatostatin activity.

### BACKGROUND OF THE INVENTION

Somatostatin is a peptide hormone that regulates the endocrine system and affects neurotransmission and cell proliferation via interaction with G-protein-coupled somatostatin receptors and inhibition of the release of numerous secondary hormones. Six subtype somatostatin receptor proteins have been identified (SSTR1, SSTR2a, SSTR2b, SSTR3, SSTR4, SSTR5) and are encoded by five different somatostatin receptor genes. Modulation of a particular subtype somatostatin receptor, or combination thereof, is attractive for the treatment of conditions, diseases, or disorders that would benefit from modulating somatostatin activity.

### SUMMARY OF THE INVENTION

In one aspect, described herein is a method of treating acromegaly in a human comprising orally administering to the human with acromegaly a daily dose of 3-[4-(4-amino-piperidin-1-yl)-3-(3,5-difluoro-phenyl)-quinolin-6-yl]-2-hydroxy-benzonitrile (Compound A), or a pharmaceutically acceptable salt, or solvate thereof, sufficient to achieve a trough blood plasma concentration of Compound A of at least about 20 ng/mL, at least about 25 ng/mL, at least about 30 ng/mL, at least about 35 ng/mL, or at least about 40 ng/mL. In some embodiments, the trough blood plasma concentration of Compound A is about 20 ng/mL, about 21 ng/mL, about 22 ng/mL, about 23 ng/mL, about 24 ng/mL, about 25 ng/mL, about 26 ng/mL, about 27 ng/mL, about 28 ng/mL, about 29 ng/mL, about 30 ng/mL, about 31 ng/mL, about 32 ng/mL, about 33 ng/mL, about 34 ng/mL, about 35 ng/mL, about 36 ng/mL, about 37 ng/mL, about 38 ng/mL, about 39 ng/mL, or about 40 ng/mL.

In another aspect, described herein is a method of treating acromegaly in a human comprising: orally administering to the human with acromegaly a daily dose of Compound A, or a pharmaceutically acceptable salt, or solvate thereof; determining the blood plasma trough concentration of Compound A in the human; and increasing the daily dose of Compound A, or a pharmaceutically acceptable salt, or solvate thereof if the human does not have at least a threshold trough blood plasma concentration of Compound A. In some embodiments, the threshold trough blood plasma concentration of Compound A is about 20 ng/mL, about 21 ng/mL, about 22 ng/mL, about 23 ng/mL, about 24 ng/mL, about 25 ng/mL, about 26 ng/mL, about 27 ng/mL, about 28 ng/mL, about 29 ng/mL, about 30 ng/mL, about 31 ng/mL, about 32 ng/mL, about 33 ng/mL, about 34 ng/mL, about 35 ng/mL, about 36 ng/mL, about 37 ng/mL, about 38 ng/mL, about 39 ng/mL, about 40 ng/mL, about 41 ng/mL, about 42 ng/mL, about 43 ng/mL, about 44 ng/mL, about 45 ng/mL, about 46 ng/mL, about 47 ng/mL, about 48 ng/mL, about 49 ng/mL, about 50 ng/mL, about 51 ng/mL, about 52 ng/mL, about 53 ng/mL, about 54 ng/mL, about 55 ng/mL, about 56 ng/mL, about 57 ng/mL, about 58 ng/mL, about 59 ng/mL, or about 60 ng/mL. In some embodiments, the threshold trough blood plasma concentration of Compound A is about 20 ng/mL, about 21 ng/mL, about 22 ng/mL, about 23 ng/mL, about 24 ng/mL, about 25 ng/mL, about 26 ng/mL, about 27 ng/mL, about 28 ng/mL, about 29 ng/mL, about 30 ng/mL, about 31 ng/mL, about 32 ng/mL, about 33 ng/mL, about 34 ng/mL, about 35 ng/mL, about 36 ng/mL, about 37 ng/mL, about 38 ng/mL, about 39 ng/mL, or about 40 ng/mL. In some embodiments, increasing the daily dose of Compound A, or a pharmaceutically acceptable salt, or solvate thereof comprises increasing the daily dose by an amount equivalent to about 10 mg of Compound A-monohydrochloride.

In some embodiments, the daily dose of Compound A, or a pharmaceutically acceptable salt, or solvate thereof is equivalent to about 10 mg/day to about 80 mg/day of Compound A-monohydrochloride. In some embodiments, the daily dose of Compound A, or a pharmaceutically acceptable salt, or solvate thereof is equivalent to about 20 mg/day to about 60 mg/day of Compound A-monohydrochloride. In some embodiments, the daily dose of Compound A, or a pharmaceutically acceptable salt, or solvate thereof is equivalent to about 10 mg/day, about 20 mg/day, about 30 mg/day, about 40 mg/day, about 50 mg/day, about 60 mg/day, about 70 mg/day, or about 80 mg/day of Compound A-monohydrochloride. In some embodiments, the daily dose of Compound A, or a pharmaceutically acceptable salt, or solvate thereof is equivalent to 20 mg/day, about 30 mg/day, about 40 mg/day, about 50 mg/day, or about 60 mg/day of Compound A-monohydrochloride.

In another aspect, described herein is a method of treating acromegaly in a human comprising improving serum insulin-like growth factor-1 (IGF-1) concentrations, growth hormone (GH) concentrations, or both, wherein the method comprises orally administering once daily to the human with acromegaly a pharmaceutical composition comprising Compound A, or a pharmaceutically acceptable salt, or solvate thereof. In some embodiments, improvements in serum insulin-like growth factor-1 (IGF-1) concentrations, growth hormone (GH) concentrations, or both comprises reductions in IGF-1 concentrations, GH concentrations, or both. In some embodiments, improving serum insulin-like growth factor-1 (IGF-1) concentrations comprises achieving IGF-1 times upper limit of normal (ULN) of less than about 2.5. In some embodiments, improvements in serum insulin-like growth factor-1 (IGF-1) concentrations, growth hormone (GH) concentrations, or both comprises achieving a trough blood plasma concentration of Compound A of at least about 20 ng/mL, at least about 25 ng/mL, at least about 30 ng/mL, at least about 35 ng/mL, or at least about 40 ng/mL. In some embodiments, improvements in serum insulin-like growth factor-1 (IGF-1) concentrations, growth hormone (GH) concentrations, or both comprises achieving a trough blood plasma concentration of Compound A is about 20 ng/mL, about 21 ng/mL, about 22 ng/mL, about 23 ng/mL, about 24 ng/mL, about 25 ng/mL, about 26 ng/mL, about 27 ng/mL, about 28 ng/mL, about 29 ng/mL, about 30 ng/mL, about 31 ng/mL, about 32 ng/mL, about 33 ng/mL, about 34 ng/mL, about 35 ng/mL, about 36 ng/mL, about 37 ng/mL, about 38 ng/mL, about 39 ng/mL, or about 40 ng/mL. In some embodiments, improving serum insulin-like growth factor-1 (IGF-1) concentrations, growth hormone (GH) concentrations, or both comprises administering a daily dose of Compound A, or a pharmaceutically acceptable salt, or solvate thereof equivalent to about 10 mg/day to about 80 mg/day of Compound A-monohydrochloride. In some embodiments, improving serum insulin-like growth factor-1 (IGF-1) concentrations, growth hormone (GH) concentrations, or both comprises administering a daily dose of Compound A, or a pharmaceutically acceptable salt, or solvate thereof equivalent to about 10 mg/day, about 20 mg/day, about 30 mg/day, about 40 mg/day, about 50 mg/day, about 60 mg/day, about 70 mg/day, or about 80 mg/day of Compound A-monohydrochloride.

In some embodiments, Compound A, or a pharmaceutically acceptable salt, or solvate thereof is orally administered by following a titration schedule.

In a further aspect, described herein is a method of treating acromegaly in a human comprising orally administering to the human with acromegaly a pharmaceutical composition comprising Compound A, or a pharmaceutically acceptable salt, or solvate thereof, via a titration schedule. In some embodiments, the titration schedule comprises daily administration of an initial dose of Compound A, or a pharmaceutically acceptable salt, or solvate thereof, for an initial period of time followed by daily administration of a dose of Compound A, or a pharmaceutically acceptable salt, or solvate thereof, that is higher than the initial dose. In some embodiments, the initial period of time comprises one day, about one week, about two weeks, about three weeks, about four weeks, about five weeks, about six weeks, about seven weeks, or about eight weeks. In some embodiments, the initial period of time comprises about two weeks.

In some embodiments, the titration schedule comprises the up-titration, or down-titration followed by an optional re-up-titration of Compound A, or a pharmaceutically acceptable salt, or solvate thereof. In some embodiments, the titration schedule comprises administering Compound A, or a pharmaceutically acceptable salt, or solvate thereof, at an initial dose for about one week to about four weeks and, provided that the patient tolerates the initial dose, increasing the dose by an amount equal to a first incremental value or provided that the patient does not tolerate the initial dose, decreasing the dose by an amount equal to a first incremental value.

In some embodiments, the titration schedule further comprises: administering Compound A, or a pharmaceutically acceptable salt, or solvate thereof, at the increased dose for about one week to about four weeks and provided that the patient tolerates the increased dose, further increasing the dose by an amount equal to a second incremental value; or administering Compound A, or a pharmaceutically acceptable salt, or solvate thereof, at the decreased dose for about one week to about four weeks and provided that the patient tolerates the decreased dose, optionally increasing the dose by an amount equal to a second incremental value. In some embodiments, the initial dose of Compound A, or a pharmaceutically acceptable salt, or solvate thereof is equivalent to about 10 mg/day or about 20 mg/day of Compound A-monohydrochloride. In some embodiments, the first incremental is equal to an amount of Compound A, or a pharmaceutically acceptable salt, or solvate thereof that is equivalent to about 10 mg of Compound A-monohydrochloride. In some embodiments, the first incremental is equal to the second incremental value. In some embodiments, the method further comprises: assessing the serum insulin-like growth factor-1 (IGF-1) concentrations of the human with acromegaly prior to each dose increase; and increasing the daily dose amount of Compound A, or a pharmaceutically acceptable salt, or solvate thereof, if the serum IGF-1 concentrations is above upper limit of normal (ULN). In some embodiments, the method further comprises: assessing the serum insulin-like growth factor-1 (IGF-1) concentrations of the human with acromegaly prior to each dose increase; and increasing the daily dose amount of Compound A, or a pharmaceutically acceptable salt, or solvate thereof, if the serum IGF-1 concentrations is about 1.1 times, about 1.2 times, about 1.3 times, about 1.4 times, about 1.5 times, about 1.6 times, about 1.7 times, about 1.8 times, about 1.9 times, about 2 times, about 2.1 times, about 2.2 times, about 2.3 times, about 2.4 times, about 2.5 times, or greater than 2.5 times upper limit of normal (ULN).

In some embodiments, the titration schedule is repeated until an optimized dose is obtained. In some embodiments, the optimized dose is equivalent to about 30 mg/day, about 40 mg/day, about 50 mg/day, about 60 mg/day, about 70 mg/day, or about 80 mg/day of Compound A-monohydrochloride. In some embodiments, the optimized dose provides a steady state plasma trough concentration of Compound A of about 20 ng/mL, about 21 ng/mL, about 22 ng/mL, about 23 ng/mL, about 24 ng/mL, about 25 ng/mL, about 26 ng/mL, about 27 ng/mL, about 28 ng/mL, about 29 ng/mL, about 30 ng/mL, about 31 ng/mL, about 32 ng/mL, about 33 ng/mL, about 34 ng/mL, about 35 ng/mL, about 36 ng/mL, about 37 ng/mL, about 38 ng/mL, about 39 ng/mL, or about 40 ng/mL.

In another aspect, described herein is a method of treating acromegaly in a human comprising orally administering to the human with acromegaly a pharmaceutical composition comprising Compound A, or a pharmaceutically acceptable salt, or solvate thereof, wherein the human has not responded to, tolerated therapy, or lost response to treatment with a somatostation analog. In some embodiments, the somatostation analog is octreotide, lanreotide, or pasireotide.

In some embodiments, Compound A, or a pharmaceutically acceptable salt, or solvate thereof is orally administered by following a titration schedule.

In yet another aspect, described herein is a method of evaluating the clinical response to treatment with Compound A, or a pharmaceutically acceptable salt, or solvate thereof, in a subject with acromegaly comprising: (a) assessing serum insulin-like growth factor-1 (IGF-1) concentrations of the subject with acromegaly prior to the initiation of treatment with Compound A; (b) administering Compound A, or a pharmaceutically acceptable salt, or solvate thereof, at an initial daily dose for an initial period of time; (c) re-assessing the serum IGF-1 concentrations of the subject with acromegaly; and (d) continuing the daily administrations of Compound A, or a pharmaceutically acceptable salt, or solvate thereof, and optionally increasing the dialy dose amount of Compound A, or a pharmaceutically acceptable salt, or solvate thereof, if the serum IGF-1 concentrations in step (a) is higher than the serum IGF-1 concentrations of step (c) or optionally discontinuing the daily administrations of the Compound A, or a pharmaceutically acceptable salt, or solvate thereof, if the serum IGF-1 concentrations in step (c) is substantially similar to the serum IGF-1 concentrations in step (a). In some embodiments, the initial period of time is about two weeks, about three weeks, or about four weeks. In some embodiments, the initial period of time is about two weeks.

In some embodiments, Compound A, or a pharmaceutically acceptable salt, or solvate thereof, is administered to the subject by following a titration schedule. In some embodiments, the titration schedule comprises one or more cycles of: administration of Compound A, or a pharmaceutically acceptable salt, or solvate thereof, at a first daily amount for a period of about two weeks, followed by: administration of Compound A, or a pharmaceutically acceptable salt, or solvate thereof, at an increased daily amount or administration of Compound A, or a pharmaceutically acceptable salt, or solvate thereof.

In some embodiments, the daily dose of Compound A, or a pharmaceutically acceptable salt, or solvate thereof, is increased if the serum IGF-1 concentrations is about 1.1 times, about 1.2 times, about 1.3 times, about 1.4 times, about 1.5 times, about 1.6 times, about 1.7 times, about 1.8 times, about 1.9 times, about 2 times, about 2.1 times, about 2.2 times, about 2.3 times, about 2.4 times, about 2.5 times, or greater than 2.5 times upper limit of normal (ULN).

In some embodiments, the daily dose of Compound A, or a pharmaceutically acceptable salt, or solvate thereof, is increased at an incremental dose amount that is equivalent to about 10 mg/day of Compound A-monohydrochloride. In some embodiments, the daily dose of Compound A, or a pharmaceutically acceptable salt, or solvate thereof, is increased at a frequency of about two weeks.

In some embodiments, the cycles of incremental dose increases are repeated until an optimized dose is obtained. In some embodiments, the optimized dose is equivalent to about 30 mg/day, about 40 mg/day, about 50 mg/day, about 60 mg/day, about 70 mg/day, or about 80 mg/day of Compound A-monohydrochloride. In some embodiments, the optimized dose provides a steady state plasma trough concentration of Compound A of about 20 ng/mL, about 21 ng/mL, about 22 ng/mL, about 23 ng/mL, about 24 ng/mL, about 25 ng/mL, about 26 ng/mL, about 27 ng/mL, about 28 ng/mL, about 29 ng/mL, about 30 ng/mL, about 31 ng/mL, about 32 ng/mL, about 33 ng/mL, about 34 ng/mL, about 35 ng/mL, about 36 ng/mL, about 37 ng/mL, about 38 ng/mL, about 39 ng/mL, or about 40 ng/mL. In some embodiments, the optimized dose maintains a normal serum IGF-1 concentration in the subject.

In some embodiments, a normal serum IGF-1 concentration in the subject is less than about 1 times upper limit of normal (ULN).

In another aspect, described herein is a method of treating acromegaly in a human comprising orally administering to the human with acromegaly a pharmaceutical composition comprising 3-[4-(4-amino-piperidin-1-yl)-3-(3,5-difluoro-phenyl)-quinolin-6-yl]-2-hydroxy-benzonitrile (Compound A), or a pharmaceutically acceptable salt, or solvate thereof; wherein the human with acromegaly was previously treated with a somatostation analog; and wherein treatment is initiated at a daily dose equivalent to about 40 mg/day of Compound A-monohydrochloride. In some embodiments, the human with acromegaly responded to and tolerated treatment with a somatostation analog. In some embodiments, the somatostation analog is octreotide, lanreotide, or pasireotide. In some embodiments, acromegaly signs and symptoms were previously controlled on octreotide or lanreotide depot monotherapy. In some embodiments, control of acromegaly signs and symptoms on octreotide or lanreotide depot monotherapy comprises IGF-1≤1.0×ULN. In some embodiments, IGF-1 levels and acromegaly signs and symptoms are assessed at a frequency of about one month. In some embodiments, the daily dose of Compound A, or a pharmaceutically acceptable salt, or solvate thereof, is increased to a daily dose equivalent to about 60 mg/day of Compound A-monohydrochloride if IGF-1>0.9×ULN. In some embodiments, if the human does not tolerate the daily dose amount of Compound A, or a pharmaceutically acceptable salt, or solvate thereof, then the daily dose is decreased by an amount equivalent to about 20 mg/day of Compound A-monohydrochloride.

In another aspect, described herein is a method of treating acromegaly in a human comprising orally administering to the human with acromegaly a pharmaceutical composition comprising 3-[4-(4-amino-piperidin-1-yl)-3-(3,5-difluoro-phenyl)-quinolin-6-yl]-2-hydroxy-benzonitrile (Compound A), or a pharmaceutically acceptable salt, or solvate thereof; wherein the human with acromegaly is treatment naïve; or wherein the human with acromegaly was untreated for acromagly within the last 4 months; or wherein the human with acromegaly was previously treated with a somatostation analog and the treatment with the somatostatin anolog is terminated and a sufficient period of time lapses to allow the somatostatin anolog to washout of the human; and wherein treatment is initiated at a daily dose equivalent to about 20 mg/day of Compound A-monohydrochloride. In some embodiments, the somatostation analog is octreotide, lanreotide, or pasireotide. In some embodiments, the daily dose of Compound A, or a pharmaceutically acceptable salt, or solvate thereof, is increased to a daily dose equivalent to about 40 mg/day of Compound A-monohydrochloride over a period of about 1 week, about 2 weeks, about 3 weeks, about 4 weeks, about 5 weeks, about 6 weeks, about 7 weeks, about 8 weeks, or about 9 weeks. In some embodiments, IGF-1 levels and acromegaly signs and symptoms are assessed at a frequency of about one month. In some embodiments, the daily dose of Compound A, or a pharmaceutically acceptable salt, or solvate thereof, is increased to a daily dose equivalent to about 60 mg/day of Compound A-monohydrochloride if IGF-1>0.9×ULN. In some embodiments, if the human with acromegaly does not tolerate the daily dose amount of Compound A, or a pharmaceutically acceptable salt, or solvate thereof, then the daily dose is decreased by an amount equivalent to about 20 mg/day of Compound A-monohydrochloride. In some embodiments, the daily dose provides a steady state plasma trough concentration of Compound A of about 20 ng/mL, about 21 ng/mL, about 22 ng/mL, about 23 ng/mL, about 24 ng/mL, about 25 ng/mL, about 26 ng/mL, about 27 ng/mL, about 28 ng/mL, about 29 ng/mL, about 30 ng/mL, about 31 ng/mL, about 32 ng/mL, about 33 ng/mL, about 34 ng/mL, about 35 ng/mL, about 36 ng/mL, about 37 ng/mL, about 38 ng/mL, about 39 ng/mL, or about 40 ng/mL. In some embodiments, treatment with Compound A is discontinued if IGF-1 levels remain above the upper normal limit after treatment with a daily dose amount of Compound A-monohydrochloride, or solvate, equivalent to about 60 mg/day or more of Compound A-monohydrochloride.

In some embodiments, treatment with Compound A, or a pharmaceutically acceptable salt, or solvate thereof, is discontinued if the human does not tolerate Compound A, Compound A-monohydrochloride, or solvate thereof.

In some embodiments, the daily dose of Compound A, or a pharmaceutically acceptable salt, or solvate thereof, is increased at an incremental dose amount that is equivalent to about 10 mg/day of Compound A-monohydrochloride.

In some embodiments, in any of the methods of treatment with Compound A described herein, the method further comprises measuring the serum glucose levels in the subject. In some embodiments, the serum glucose levels in the subject are measured when: treatment with Compound A, or a pharmaceutically acceptable salt, or solvate thereof, is initiated; when the dose of Compound A, or a pharmaceutically acceptable salt, or solvate thereof, is adjusted; or both. In some embodiments, the method further comprises measuring the serum glucose levels in the subject when: treatment with Compound A, or a pharmaceutically acceptable salt, or solvate thereof, is initiated; when the dose of Compound A, or a pharmaceutically acceptable salt, or solvate thereof, is increased; or both. In some embodiments, measuring the serum glucose levels in the subject comprises administering an oral glucose tolerance test (OGTT). In some embodiments, antidiabetic treatment is optionally initiated, or antidiabetic treatment is optonally adjusted if peak serum glucose concentrations in the subject are >150 mg/dl as measured with an oral glucose tolerance test (OGTT).

In some embodiments, Compound A, or a pharmaceutically acceptable salt, or solvate thereof, is administered in the form of one or more capsules or tablets.

In some embodiments, Compound A, or a pharmaceutically acceptable salt, or solvate thereof, is administered in the form of one or more capsules. In some embodiments, Compound A, or a pharmaceutically acceptable salt, or solvate thereof, is administered in the form of one or more capsules, wherein the capsules are the HMG capsules described herein.

In some embodiments, Compound A, or a pharmaceutically acceptable salt, or solvate thereof, is administered in the form of one or more tablets. In some embodiments, Compound A, or a pharmaceutically acceptable salt, or solvate thereof, is administered in the form of one or more tablets, wherein the tablets are as described herein.

In some embodiments, Compound A, or a pharmaceutically acceptable salt, or solvate thereof, is administered in the form of one or more tablets, wherein each tablet comprises: a spray-dried solid dispersion of Compound A monohydrochloride, or solvate thereof; one or more additional pharmaceutically acceptable ingredients; and optionally one or more film coating agents. In some embodiments, the spray-dried solid dispersion comprises: (a) Compound A monohydrochloride, or solvate thereof; and (b) a pharmaceutically acceptable polymer; wherein Compound A monohydrochloride, or solvate thereof, is dispersed in a polymer matrix formed from the pharmaceutically acceptable polymer. In some embodiments, the one or more pharmaceutically acceptable ingredients are selected from the group consisting of one or more diluents, one or more disintegrants, one or more lubricants, and one or more glidants. In some embodiments, the one or more pharmaceutically acceptable ingredients comprise microcrystalline cellulose, mannitol, pregelatinized starch croscarmellose sodium crospovidone, sodium chloride, 1:1 sodium chloride:potassium chloride, colloidal silicon dioxide, and magnesium stearate.

In some embodiments, each tablet comprises about 2% by weight to about 20% by weight of Compound A monohydrochloride, or solvate thereof. In some embodiments, each tablet comprises about 2% by weight, about 3% by weight, about 4% by weight, about 5% by weight, about 6% by weight, about 7% by weight, about 8% by weight, about 9% by weight, about 10% by weight, about 11% by weight, about 12% by weight, about 13% by weight, about 14% by weight, or about 15% by weight of Compound A monohydrochloride, or solvate thereof. In some embodiments, each tablet comprises about 10% by weight to about 35% by weight of the polymer matrix formed from the pharmaceutically acceptable polymer. In some embodiments, each tablet comprises: about 2% by weight to about 15% by weight of Compound A monohydrochloride, or solvate thereof, dispersed in a polymer matrix formed from a pharmaceutically acceptable polymer, wherein the dispersed Compound A monohydrochloride, or solvate thereof, in the polymer matrix is about about 20% by weight to about 35% by weight of the tablet; about 40% by weight to about 80% by weight of one or more pharmaceutically acceptable ingredients selected from the group consisting of one or more diluents, one or more disintegrants, one or more lubricants, and one or more glidants; and optionally less than about 5% by weight of one or more film coating agents.

In some embodiments, the tablet comprises: about 20% by weight to about 40% of a spray dried dispersion of Compound A monohydrochloride, or solvate thereof, dispersed in a polymer matrix formed from a pharmaceutically acceptable polymer; about 60% by weight to about 80% by weight of one or more pharmaceutical acceptable ingredients selected from the group consisting of one or more diluents, one or more disintegrants, one or more disintegrant aids, one or more lubricants, and one or more glidants; and optionally less than about 5% by weight of one or more film coating agents.

In some embodiments, the spray dried dispersion comprises an about 15/85 to about 35/65 ratio of Compound A monohydrochloride, or solvate thereof to a polymer matrix of hydroxypropyl methyl cellulose acetate succinate (HPMCAS), or polyvinylpyrrolidone polyvinyl acetate copolymers (PVP/VA).

In some embodiments, each tablet comprises: about 20% by weight to about 35% of by weight a spray dried dispersion of Compound A monohydrochloride, or solvate thereof, dispersed in a polymer matrix formed from a pharmaceutically acceptable polymer; wherein the spray dried dispersion comprises an about 15/85 to about 35/65 ratio of Compound A monohydrochloride, or solvate thereof to a polymer matrix of hydroxypropyl methyl cellulose acetate succinate (HPMCAS), or polyvinylpyrrolidone polyvinyl acetate copolymers (PVP/VA); about 60% by weight to about 80% by weight of one or more pharmaceutical acceptable ingredients selected from the group consisting of microcrystalline cellulose, mannitol, pregelatinized starch, croscarmellose sodium, crospovidone, sodium chloride, 1:1 sodium chloride:potassium chloride, silicon dioxide, and magnesium stearate; optionally less than about 5% by weight of one or more film coating agents.

In some embodiments, each tablet comprises: about 20%, about 21%, about 22%, about 23%, about 24%, about 25%, about 26%, about 27%, about 28%, about 29%, about 30%, about 31%, about 32%, about 33%, about 34%, or about 35% by weight of a spray dried dispersion of Compound A monohydrochloride, or solvate thereof, dispersed in a polymer matrix formed from a pharmaceutically acceptable polymer; wherein the spray dried dispersion comprises an about 15/85 or about 35/65 ratio of Compound A monohydrochloride, or solvate thereof to a polymer matrix of hydroxypropyl methyl cellulose acetate succinate (HPMCAS), or polyvinylpyrrolidone polyvinyl acetate copolymers (PVP/VA); about 60% by weight to about 80% by weight of one or more pharmaceutical acceptable ingredients selected from the group consisting of one or more diluents, one or more disintegrants, one or more disintegrant aids, one or more lubricants, and one or more glidants; and optionally less than about 5% by weight of one or more film coating agents.

In some embodiments, each tablet comprises: about 20%, about 21%, about 22%, about 23%, about 24%, about 25%, about 26%, about 27%, about 28%, about 29%, about 30%, about 31%, about 32%, about 33%, about 34%, or about 35% by weight of a spray dried dispersion of Compound A monohydrochloride, or solvate thereof, dispersed in a polymer matrix formed from a pharmaceutically acceptable polymer; wherein the spray dried dispersion comprises an about 15/85 or about 35/65 ratio of Compound A monohydrochloride, or solvate thereof to a polymer matrix of hydroxypropyl methyl cellulose acetate succinate (HPMCAS), or polyvinylpyrrolidone polyvinyl acetate copolymers (PVP/VA); about 60% by weight to about 80% by weight of one or more pharmaceutical acceptable ingredients selected from the group consisting of microcrystalline cellulose, mannitol, pregelatinized starch, croscarmellose sodium, crospovidone, sodium chloride, 1:1 sodium chloride:potassium chloride, silicon dioxide, and magnesium stearate; optionally less than about 5% by weight of one or more film coating agents.

In some embodiments, each tablet comprises: about 2% by weight to about 15% by weight of Compound A monohydrochloride, or solvate thereof, dispersed in a polymer matrix formed from a pharmaceutically acceptable polymer, wherein the dispersed Compound A monohydrochloride, or solvate thereof, in the polymer matrix is about 20% by weight to about 35% by weight of the tablet; about 40% by weight to about 80% by weight of one or more pharmaceutically acceptable ingredients selected from the group consisting of one or more diluents, one or more disintegrants, one or more lubricants, and one or more glidants; and optionally less than about 5% by weight of one or more film coating agents.

In some embodiments, each tablet comprises: about 20% by weight to about 40% of a spray dried dispersion of Compound A monohydrochloride, or solvate thereof, dispersed in a polymer matrix formed from a pharmaceutically acceptable polymer; about 60% by weight to about 80% by weight of one or more pharmaceutical acceptable ingredients selected from the group consisting of one or more diluents, one or more disintegrants, one or more disintegrant aids, one or more lubricants, and one or more glidants; and optionally less than about 5% by weight of one or more film coating agents.

In some embodiments, the spray dried dispersion comprises an about 15/85 to about 35/65 ratio of Compound A monohydrochloride, or solvate thereof to a polymer matrix of hydroxypropyl methyl cellulose acetate succinate (HPMCAS), or polyvinylpyrrolidone polyvinyl acetate copolymers (PVP/VA).

In some embodiments, each tablet comprises: about 20% by weight to about 35% of by weight a spray dried dispersion of Compound A monohydrochloride, or solvate thereof, dispersed in a polymer matrix formed from a pharmaceutically acceptable polymer; wherein the spray dried dispersion comprises an about 15/85 to about 35/65 ratio of Compound A monohydrochloride, or solvate thereof to a polymer matrix of hydroxypropyl methyl cellulose acetate succinate (HPMCAS), or polyvinylpyrrolidone polyvinyl acetate copolymers (PVP/VA); about 60% by weight to about 80% by weight of one or more pharmaceutical acceptable ingredients selected from the group consisting of one or more diluents, one or more disintegrants, one or more disintegrant aids, one or more lubricants, and one or more glidants; and optionally less than about 5% by weight of one or more film coating agents.

In some embodiments, each tablet comprises about 10 mg, about 20 mg, about 40 mg, about 60 mg or about 80 mg of Compound A monohydrochloride, or solvate thereof. In some embodiments, each tablet comprises about 10 mg of Compound A monohydrochloride, or solvate thereof. In some embodiments, each tablet comprises about 20 mg of Compound A monohydrochloride, or solvate thereof. In some embodiments, each tablet comprises about 30 mg of Compound A monohydrochloride, or solvate thereof. In some embodiments, each tablet comprises about 40 mg of Compound A monohydrochloride, or solvate thereof. In some embodiments, each tablet comprises about 50 mg of Compound A monohydrochloride, or solvate thereof. In some embodiments, each tablet comprises about 60 mg of Compound A monohydrochloride, or solvate thereof. In some embodiments, each tablet comprises about 70 mg of Compound A monohydrochloride, or solvate thereof. In some embodiments, each tablet comprises about 80 mg of Compound A monohydrochloride, or solvate thereof.

In some embodiments, Compound A, or a pharmaceutically acceptable salt, or solvate thereof, is administered once daily at least 30 minutes before a meal. In some embodiments, Compound A, or a pharmaceutically acceptable salt, or solvate thereof, is administered once daily at least 60 minutes before a meal. In some embodiments, Compound A is administered once daily on an empty stomach. In some embodiments, Compound A is administered at least 30 minutes before a meal. In some embodiments, Compound A is administered at least 60 minutes before a meal. In some embodiments, Compound A is administered at least 180 minutes after a meal. In some embodiments, Compound A is administered at least 60 minutes before a meal and at least 180 minutes after a meal. In some embodiments, Compound A is administered before bedtime. In some embodiments, Compound A is administered once daily with a glass of water on an empty stomach at least 30 minutes before a meal.

In some embodiments, the bioavailability of Compound A monohydrochloride, or solvate thereof, is not substantially affected by the coadministration of proton pump inhibitors, histamine H2-receptor antagonists, or antacids. In some embodiments, Compound A, or a pharmaceutically acceptable salt, or solvate thereof, is not co-administered with a drug that alters the pH of the upper gastrointestinal (GI) tract.

In some embodiments, if Compound A, or a pharmaceutically acceptable salt, or solvate thereof, is co-administered with a drug that alters the pH of the upper gastrointestinal (GI) tract, then the daily dose amount of Compound A, or a pharmaceutically acceptable salt, or solvate thereof, that is administered is increased.

In some embodiments, the daily dose amount of Compound A, or a pharmaceutically acceptable salt, or solvate thereof, is increased by an amount equivalent to about 10 mg/day or about 20 mg/day of Compound A-monohydrochloride.

In some embodiments, the drug that alters the pH of the upper gastrointestinal (GI) tract comprises proton pump inhibitors, histamine H2-receptor antagonists, or antacids.

In some embodiments, Compound A, or a pharmaceutically acceptable salt, or solvate thereof, is co-administered with a dopamine agonist. In some embodiments, the dopamine agonist is cabergoline, or a pharmaceutically acceptable salt thereof. In some embodiments, the dopamine agonist is cabergoline and is administered at a dose and frequency of about 0.5 mg/week to about 0.5 mg/day

In one aspect, described herein is a method of treating acromegaly or neuroendocrine tumors, or both, in a human comprising orally administering to the human with acromegaly or neuroendocrine tumors any one of the spray-dried dispersion tablets described herein.

In some embodiments, the tablet is administered once daily. In some embodiments, the tablet is administered at least 30 minutes before a meal. In some embodiments, the tablet is administered at least 60 minutes before a meal. In some embodiments, the tablet is administered at least 180 minutes after a meal. In some embodiments, the tablet is administered at least 60 minutes before a meal and at least 180 minutes after a meal. In some embodiments, the tablet is administered on an empty stomach. In some embodiments, the tablet is administered with a glass of water on an empty stomach at least 30 minutes before a meal. In some embodiments, the tablet is administered before bedtime. In some embodiments, the bioavailability of Compound A monohydrochloride, or solvate thereof, from the tablet is not substantially affected by the coadministration of proton pump inhibitors, histamine H2-receptor antagonists, or antacids.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### BRIEF DESCRIPTION OF THE DRAWINGS

FIG. 1. Illustrates the observed dose proportionality in humans administered the HMG capsule formulation or the SDD tablet formulation of Compound A-HCl.

FIG. 2. Illustrates the performance of the HMG capsule formulation and the SDD tablet formulation of Compound A-HCl in dogs with or without pentagastrin pretreatment.

FIG. 3. Illustrates the hormone levels observed in primary analysis population of the Acrobat Edge clinical trial.

FIG. 4. Illustrates the evidence of a dose response observed in the Acrobat Edge and Evolve clinical trials.

FIG. 5. Illustrates the estimated trough plasma paltusotine concentrations needed for therapeutic effect and projections of the dose proportional effects projected for the SDD tablet formulations.

FIG. 6. Illustrates the projected trough plasma paltusotine concentrations of acromegaly patients taking PPIs who are on 60 mg SDD tablets (1 hour post dose fast) compared to projected trough plasma paltusotine concentrations of acromegaly patients not taking PPIs and trough plasma paltusotine concentrations of acromegaly patients who are on 40 mg HMG capsule formualtions (2 h post dose fast).

FIG. 7a. Shows the interim results from the Acrobat Advance study for the subset of patients on paltusotine monotherapy.

FIG. 7b. Illustrates the interim results from the Acrobat Advance study for the subset of patients previously enrolled in the Evolve study.

FIG. 8a. Illustrates the interim results from the Acrobat Advance study for all patients enrolled in the study.

FIG. 8b. Illustrates the interim results from the Acrobat Advance study for the subset of patients on therapy with paltusotine and cabergoline.

### DETAILED DESCRIPTION OF THE INVENTION

Somatostatin (SST), also known as somatotropin release inhibiting factor (SRIF) was initially isolated as a 14-amino acid peptide from ovine hypothalamii (Brazeau et al., Science 179, 77-79, 1973). An N-terminal extended 28-amino acid peptide with similar biological activity to 14-amino acid somatostatin was subsequently isolated (Pradayrol et, al., FEBS Letters, 109, 55-58, 1980; Esch et al., Proc. Natl. Acad. Sci. USA, 77, 6827-6831, 1980). SST is a regulatory peptide produced by several cell types in response to other neuropeptides, neurotransmitters, hormones, cytokines, and growth factors. SST acts through both endocrine and paracrine pathways to affect its target cells. Many of these effects are related to the inhibition of secretion of other hormones, most notably growth hormone (GH). They are produced by a wide variety of cell types in the central nervous system (CNS) and gut, and have multiple functions including modulation of secretion of growth hormone (GH), insulin, glucagon, as well as many other hormones that are anti-proliferative.

These pleotropic actions of somatostatins are mediated by six somatostatin receptor proteins (SSTR1, SSTR2a, SSTR2b, SSTR3, SSTR4, SSTR5). The six somatostatin receptor proteins are encoded by five different somatostatin receptor genes (Reisine and Bell, Endocr Rev. 16, 427-442, 1995; Patel and Srikant, Trends Endocrinol Metab 8, 398-405, 1997). All the receptors are members of the class-A subgroup of the G protein-coupled receptor (GPCR) superfamily. SST2A receptor is the most widely expressed subtype in human tumors and is the dominant receptor by which GH secretion is suppressed. Unless otherwise stated, the term SSTR2 means SSTR2a.

It is possible to selectively modulate any one of the somatostatin receptor subtypes, or combination thereof. In some embodiments, selectively modulating any one of the somatostatin receptor subtypes relative to the other somatostatin receptor subtypes, or combination thereof, is useful in a variety of clinical applications. In some embodiments, selectively modulating any one of the somatostatin receptor subtypes relative to the other somatostatin receptor subtypes reduces unwanted side effects in a variety of clinical applications.

For example, modulation of SSTR2 activity mediates the inhibition of growth hormone (GH) release from the anterior pituitary and glucagon release from pancreas. SSTR2 is also implicated in many other biological functions such as, but not limited to, cell proliferation, nociception, inflammation, and angiogenesis. In some embodiments, a selective SSTR2 modulator is used in the treatment of acromegaly, pituitary gigantism, gut neuroendocrine tumors, pain, neuropathies, nephropathies, and inflammation, as well as retinopathies resulting from aberrant blood vessel growth. In some other embodiments, a selective SSTR2 modulator is used in the treatment of arthritis, pain, cancer, inflammatory bowel disease, irritable bowel syndrome, Crohn's disease, Cushing's disease, acute lung injury, acute respiratory distress syndrome, and ophthalmic disorders such as age-related macular degeneration (AMD), diabetic retinopathy, diabetic macular edema, and Graves ophthalmology, among others.

In some embodiments, SSTR3 agonists inhibit insulin secretion. In some embodiments, SSTR4 agonists exhibit anti-inflammatory and anti-nociceptive effects. In some embodiments, SSTR5 agonists inhibit insulin secretion. In addition, SSTR5 has also been implicated to modulate the release of growth hormone.

Somatostatin peptide and its receptor subtypes are also widely expressed in the brain and disruption or diminishment of their activity is potentially involved in several psychiatric and neurodegenerative diseases. For example, concentrations of somatostatin in the cebrebral cortex and hippocampus are reduced in schizophrenics and one of the most consistent neuropathologic findings in this patient group is a deficit in cortical inhibitory intemeurons expressing somatostatin. Somatostatin is also highly expressed in brain regions associated with seizures and has also been implicated as having an important role in epilepsy. Somatostatin levels are diminished in the hippocampi of Alzheimer's and Parkinson's patients, suggesting that restoration of its signaling as a potential drug target for neurodegeneration.

In one aspect, Compound A is a selective nonpeptide SST2 biased agonist that is amenable to oral administration to a mammal in need of treatment with a somatostatin modulator.

In some embodiments, the somatostatin receptor modulator described herein has utility over a wide range of therapeutic applications. In some embodiments, the somatostatin receptor modulator described herein is used in the treatment of a variety of diseases or conditions such as, but not limited to acromegaly, pituitary gigantism, neuroendocrine tumors, retinopathies and other ophthalmic disorders, neuropathy, nephropathy, respiratory diseases, cancers, pain, neurodegenerative diseases, inflammatory diseases, as well as psychiatric and neurodegenerative disorders. In some embodiments, the somatostatin receptor modulator described herein is used in the treatment of acromegaly, neuroendocrine tumors, or both in a mammal. In some embodiments, the somatostatin receptor modulator described herein is used in the treatment of acromegaly in a mammal. In some embodiments, the somatostatin receptor modulator described herein is used in the treatment of neuroendocrine tumors. In some embodiments, the somatostatin receptor modulatory described herein is used in the treatment of pituitary gigantism in a mammal.

In some embodiments, the somatostatin receptor modulator described herein inhibits the secretion of various hormones and trophic factors in mammals. In some embodiments, the somatostatin receptor modulator described herein is used to suppress certain endocrine secretions, such as, but not limited to GH, insulin, glucagon and prolactin. The suppression of certain endocrine secretions is useful in the treatment of disorders such as acromegaly; endocrine tumors such as carcinoids, VIPomas, insulinomas and glucagonomas; or diabetes and diabetes-related pathologies, including retinopathy, neuropathy and nephropathy. In some embodiments, the somatostatin receptor modulator described herein is used to suppress exocrine secretions in the pancreas, stomach and intestines, for the treatment of disorders such as pancreatitis, fistulas, bleeding ulcers and diarrhea associated with such diseases as AIDS or cholera. Disorders involving autocrine or paracrine secretions of trophic factors such as IGF-1 (as well as some endocrine factors) which may be treated by administration of the compounds described herein include cancers of the breast, prostate, and lung (both small cell and non-small cell epidermoids), as well as hepatomas, neuroblastomas, colon and pancreatic adenocarcinomas (ductal type), chondrosarcomas, and melanomas, diabetic retinopathy, and atherosclerosis associated with vascular grafts and restenosis following angioplasty.

In some embodiments, the somatostatin receptor modulator described herein is used to suppress the mediators of neurogenic inflammation (e.g. substance P or the tachykinins), and may be used in the treatment of rheumatoid arthritis; psoriasis; topical inflammation such as is associated with sunburn, eczema, or other sources of itching; inflammatory bowel disease; irritable bowel syndrome; allergies, including asthma and other respiratory diseases In some other embodiments, the somatostatin receptor modulators described herein function as neuromodulators in the central nervous system and are useful in the treatment of Alzheimer's disease and other forms of dementia, pain, and headaches. In some embodiments, the somatostatin receptor modulator described herein provides cytoprotection in disorders involving the splanchnic blood flow, including cirrhosis and oesophagal varices.

Compound A is a somatostatin modulator that is useful in the methods of treatment described herein.

### Compound A

As used herein, Compound A refers to 3-(4-(4-amino-piperidin-1-yl)-3-(3,5-difluoro-phenyl)-quinolin-6-yl)-2-hydroxy-benzonitrile, which has the chemical structure shown below.

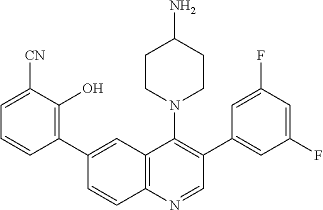 [SMILES: N#Cc1cccc(-c2ccc3ncc(-c4cc(F)cc(F)c4)c(N4CCC(N)CC4)c3c2)c1O]

Compound A is also known as “paltusotine”. Other names may be known or given to Compound A.

Compound A is a selective nonpeptide SST2 biased agonist. In clinical studies, Compound A was shown to have an estimated bioavailability of about 70% and an observed half-life of about 42 to about 50 hours. In some embodiments, Compound A is used to treat acromegaly, neuroendocrine tumors, or both. In some embodiments, Compound A is used to treat acromegaly. In some embodiments, Compound A is used to treat neuroendocrine tumors. In some embodiments, Compound A is used to treat pituitary gigantism.

In some embodiments, the free base form of Compound A is incorporated into the formulations described herein. In some embodiments, Compound A is incorporated into the formulations described herein as a pharmaceutically acceptable salt. In some embodiments, Compound A is incorporated into the formulations described herein as a pharmaceutically acceptable solvate.

“Pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich:Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts are obtained by reacting a compound disclosed herein with an acid. In some embodiments, the compound disclosed herein (i.e. free base form) is basic and is reacted with an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, Compound A is incorporated into the formulations described herein as a pharmaceutically acceptable salt form that is selected from Compound A hydrochloride and Compound A methanesulfonic acid. In some embodiments, the Compound A salt form is Compound A monohydrochloride. In some embodiments, the Compound A salt form is Compound A dihydrochloride. In some embodiments, the Compound A salt form is Compound A monomethanesulfonic acid. In some embodiments, the Compound A salt form is Compound A dimethanesulfonic acid.

In one aspect, Compound A monohydrochloride (Compound A-HCl) is incorporated into the pharmaceutical compositions described herein. Compound A monohydrochloride (Compound A-HCl), also known as 3-[4-(4-amino-piperidin-1-yl)-3-(3,5-difluoro-phenyl)-quinolin-6-yl]-2-hydroxy-benzonitrile monohydrochloride and paltusotine monohydrochloride, has the following structure:

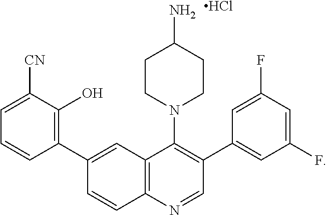 [SMILES: Cl ; N#Cc1cccc(-c2ccc3ncc(-c4cc(F)cc(F)c4)c(N4CCC(N)CC4)c3c2)c1O]

In some embodiments, the Compound A salt form is amorphous. In some embodiments, Compound A monohydrochloride is amorphous

In some embodiments, the Compound A salt form is crystalline. In some embodiments, Compound A monohydrochloride is crystalline.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

Therapeutic agents that are administrable to mammals, such as humans, must be prepared by following regulatory guidelines. Such government regulated guidelines are referred to as Good Manufacturing Practice (GMP). GMP guidelines outline acceptable contamination levels of active therapeutic agents, such as, for example, the amount of residual solvent in the final product. Preferred solvents are those that are suitable for use in GMP facilities and consistent with industrial safety concerns. Categories of solvents are defined in, for example, the International Conference on Harmonization of Technical Requirements for Registration of Pharmaceuticals for Human Use (ICH), “Impurities: Guidelines for Residual Solvents, Q3C(R3), (November 2005).

Solvents are categorized into three classes. Class 1 solvents are toxic and are to be avoided. Class 2 solvents are solvents to be limited in use during the manufacture of the therapeutic agent. Class 3 solvents are solvents with low toxic potential and of lower risk to human health. Data for Class 3 solvents indicate that they are less toxic in acute or short-term studies and negative in genotoxicity studies.

Class 1 solvents, which are to be avoided, include: benzene; carbon tetrachloride; 1,2-dichloroethane; 1,1-dichloroethene; and 1,1,1-trichloroethane.

Examples of Class 2 solvents are: acetonitrile, chlorobenzene, chloroform, cyclohexane, 1,2-dichloroethene, dichloromethane, 1,2-dimethoxyethane, N,N-dimethylacetamide, N,N-dimethylformamide, 1,4-dioxane, 2-ethoxyethanol, ethyleneglycol, formamide, hexane, methanol, 2-methoxyethanol, methylbutyl ketone, methylcyclohexane, N-methylpyrrolidine, nitromethane, pyridine, sulfolane, tetralin, toluene, 1,1,2-trichloroethene and xylene.

Class 3 solvents, which possess low toxicity, include: acetic acid, acetone, anisole, 1-butanol, 2-butanol, butyl acetate, tert-butylmethyl ether (MTBE), cumene, dimethyl sulfoxide, ethanol, ethyl acetate, ethyl ether, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, propyl acetate, and tetrahydrofuran.

Residual solvents in active pharmaceutical ingredients (APIs) originate from the manufacture of API. In most cases, the solvents are not completely removed by practical manufacturing techniques. Appropriate selection of the solvent for the synthesis of APIs may enhance the yield, or determine characteristics such as crystal form, purity, and solubility. Therefore, the solvent is a critical parameter in the synthetic process.

In some embodiments, compositions comprising Compound A-HCl include a residual amount of an organic solvent(s). In some embodiments, compositions comprising Compound A-HCl comprise a residual amount of a Class 2 or Class 3 solvent. In some embodiments, compositions comprising Compound A-HCl comprise a residual amount of a solvent selected from ethyl acetate, isopropyl acetate, tert-butylmethylether, heptane, isopropanol, methanol, acetone, dimethylformamide, tetrahydrofuran, 1,4-dioxane, 2-methyltetrahydrofuran, toluene, and ethanol.

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an agonist.

The terms “administer,” “administering”, “administration,” and the like, as used herein, refer to the methods that may be used to enable delivery of compounds or compositions to the desired site of biological action. These methods include, but are not limited to oral routes of administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound disclosed herein, or a pharmaceutically acceptable salt thereof, and a co-agent, are both administered to a patient simultaneously in the form of a single entity or dosage. The term “non-fixed combination” means that the active ingredients, e.g. a compound disclosed herein, or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “article of manufacture” and “kit” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

In some embodiments, treating acromegaly with Compound A, or a pharmaceutically acceptable salt or solvate thereof, comprises suppressing growth hormone (GH), insulin growth factor 1 (IGF-1), or both.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be effected by any method that enables delivery of the compounds to the site of action. These methods include, though are not limited to delivery via enteral routes (including oral) administration, although the most suitable route may depend upon for example the condition and disorder of the recipient.

In some embodiments, pharmaceutical compositions suitable for oral administration are presented as discrete units such as capsules or tablets each containing a predetermined amount of the active ingredient; or as a powder or granules.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets for identification or to characterize different combinations of active compound doses.

Conventional techniques to manufacture solid oral dosage forms include, but are not limited to, one or a combination of methods: (1) dry mixing, (2) direct compression, (3) milling, (4) dry or non-aqueous granulation, or (5) wet granulation. See, e.g., Lachman et al., The Theory and Practice of Industrial Pharmacy (1986). Other methods include, e.g., spray drying, pan coating, melt granulation, granulation, fluidized bed spray drying or coating (e.g., wurster coating), tangential coating, top spraying, tableting, extruding and the like.

It should be understood that in addition to the ingredients particularly mentioned above, the compounds and compositions described herein may include other agents conventional in the art having regard to the type of formulation in question, for example those suitable for oral administration may include flavoring agents.

Provided herein are tablets comprising Compound A, or a pharmaceutically acceptable salt thereof. In some embodiments, the tablet comprises: Compound A-HCl, or solvate thereof, dispersed in a polymer matrix formed from a pharmaceutically acceptable polymer; one or more pharmaceutical acceptable ingredients selected from the group consisting of one or more diluents, one or more disintegrants, one or more lubricants, and one or more glidants; and optionally one or more film coating agents.

In some embodiments, described herein is a spray-dried solid dispersion comprising (a) Compound A-HCl, or solvate thereof; and (b) a pharmaceutically acceptable polymer; wherein Compound A-HCl, or solvate thereof, is dispersed in a polymer matrix formed from the pharmaceutically acceptable polymer.

In some embodiments, described herein are tablets prepared with the spray-dried solid dispersions described herein.

### Spray-Dried Solid Dispersion (SDD)

The amorphous state for most small molecule drugs is thermodynamically unstable and, unless the glass transition temperature (Tg) is sufficiently high, also kinetically unstable. However, the amorphous state can be stabilized by dilution of the drug in an excipient matrix. When an amorphous molecule is dispersed in a high Tg matrix, low molecular mobility provides a diffusion barrier which inhibits molecular mobility that is required for phase separation upon storage. Phase separation into drug rich domains is the precursor to forming crystal nuclei and eventually widespread crystallization which results in a lost solubility advantage. In some embodiments, pharmaceutically acceptable polymers for use in preparing spray-dried solid dispersions are polymers with a high Tg. In cases when the active pharmaceutical ingredient (API) and excipient are not thermodynamically miscible with one another in the solid state, the spray-dried dispersion (SDD) is formulated such that the resulting Tg of the mixture, including absorbed water, is at least 10° C. to 20° C. greater than typical storage conditions. Further, considerations must be made with respect to water uptake during storage, either through selection of a non-hygroscopic polymer or packaging configuration, as adsorbed water will plasticize the dispersion and lower the Tg.

In some embodiments, Compound A-HCl, or solvate thereof, in the spray-dried solid dispersions described herein is substantially amorphous.

In some embodiments, the pharmaceutically acceptable polymer comprises polymers of: cellulose optionally functionalized with any combination of alkyl ethers, alkyl esters, phthalate esters; vinyl alcohol; vinyl acetate; propylene glycol; pyrrolidone; vinylpyrrolidone, oxyethylene; oxypropylene; methacrylic acid; methyl methacrylate; ethylene glycol; ethylene glycol glycerides; ethylene oxide; propylene oxide; 2-ethyl-2-oxazoline; maleic acid; methyl vinyl ether; vinyl caprolactam; or combinations thereof.

In some embodiments, the pharmaceutically acceptable polymer is hydroxypropyl methylcellulose (HPMC), hydroxypropyl methyl cellulose acetate succinate (HPMCAS), hydroxypropyl cellulose (HPC), methyl cellulose, hydroxyethyl methyl cellulose, hydroxyethyl cellulose acetate, hydroxyethyl ethyl cellulose, polyvinyl alcohol polyvinyl acetate copolymers, polyethylene glycol, polyethylene glycol polypropylene glycol copolymers, polyvinylpyrrolidone (PVP), polyvinylpyrrolidone polyvinyl acetate copolymers (PVP/VA), polyethylene polyvinyl alcohol copolymers, polyoxyethylene-polyoxypropylene block copolymers, cellulose acetate phthalate (CAP), hydroxypropyl methyl-cellulose phthalate (HPMCP), co-polymer of methacrylic acid and methyl methacrylate, polyethylene glycol glycerides composed of mono-, di- and triglycerides and mono- and diesters of polyethylene glycol, hydroxypropylcellulose, copolymers of ethylene oxide and propylene oxide blocks, poly(2-ethyl-2-oxazoline), poly(maleic acid/methyl vinyl ether), polyvinyl caprolactam-polyvinyl acetate-polyethylene glycol graft copolymer, ethylene oxide/propylene oxide tetra functional block copolymer, d-alpha tocopheryl polyethylene glycol 1000 succinate, or combinations thereof.

In some embodiments, the spray-dried dispersion further comprises a dispersion polymer. Dispersion polymers are selected from hydroxypropyl methylcellulose (HPMC), hypromellose acetate succinate (hydroxypropyl methyl cellulose acetate succinate; HPMCAS, such as HPMCAS-H, HPMCAS-L, or HPMCAS-M), hydroxypropyl cellulose (HPC), methyl cellulose, hydroxyethyl methyl cellulose, hydroxyethyl cellulose acetate, hydroxyethyl ethyl cellulose, polyvinyl alcohol polyvinyl acetate copolymers, polyethylene glycol, polyethylene glycol polypropylene glycol copolymers, polyvinylpyrrolidone (PVP), polyvinylpyrrolidone polyvinyl acetate copolymers (PVP/VA), polyethylene polyvinyl alcohol copolymers, polyoxyethylene-polyoxypropylene block copolymers, and combinations thereof.

HPMCAS is a cellulosic polymer with four types of substituents semirandomly substituted on the hydroxyls: methoxy, hydroxypropyloxy, acetate, and succinate. The polymer is available in three grades: L, M and H, based on the content of acetyl and succinoyl groups (wt %) in the HPMCAS molecule. Grade L: 5-9% by weight acetate, 14-18% by weight succinate, 20-24% by weight methoxy, 5-9% by weight hydroxypropyloxy. Grade M: 7-11% by weight acetate, 10-14% by weight succinate, 21-25% by weight methoxy, 5-9% by weight hydroxypropyloxy. Grade H: 10-14% by weight acetate, 4-8% by weight succinate, 22-26% by weight methoxy, 6-10% by weight hydroxypropyloxy.

In some embodiments, the pharmaceutically acceptable polymer is selected from PVP/VA 64, PVP 30, HPMCAS-L, HPMCAS-M, HPMCAS-H, Eudragit L100-55, poly(methacrylic acid-co-methyl methacrylate)(PMMAMA, or trade name Eudragit L100), Eudragit EPO, HPMC E15, HPMC E3, HPMC E5, HPMCP-HP55, and Soluplus.

In some embodiments, the pharmaceutically acceptable polymer is selected from PVP/VA 64 and HPMCAS-M. In some embodiments, the pharmaceutically acceptable polymer is PVP/VA 64. In some embodiments, the pharmaceutically acceptable polymer is HPMCAS-M.

In some embodiments, the weight ratio of Compound A-HCl, or solvate thereof, to the dispersion polymer is between about 1:10 and about 10:1. In some embodiments, the weight ratio of Compound A-HCl, or solvate thereof, to the dispersion polymer is between about 1:1 and about 1:10. In some embodiments, the weight ratio of Compound A-HCl, or solvate thereof, to the dispersion polymer is between about 1:3 and about 1:8. In some embodiments, the weight ratio of Compound A-HCl, or solvate thereof, to the dispersion polymer is between about 1:4 and about 1:7. In some embodiments, the weight ratio of Compound A-HCl, or solvate thereof, to the dispersion polymer is between about 1:4 and about 1:6. In some embodiments, the weight ratio of Compound A-HCl, or solvate thereof, to the dispersion polymer is between about 1:5 and about 1:6. In some embodiments, the weight ratio of Compound A-HCl, or solvate thereof, to the dispersion polymer is about 1:10. In some embodiments, the weight ratio of Compound A-HCl, or solvate thereof, to the dispersion polymer is about 1:9. In some embodiments, the weight ratio of Compound A-HCl, or solvate thereof, to the dispersion polymer is about 1:8. In some embodiments, the weight ratio of Compound A-HCl, or solvate thereof, to the dispersion polymer is about 1:7. In some embodiments, the weight ratio of Compound A-HCl, or solvate thereof, to the dispersion polymer is about 1:6. In some embodiments, the weight ratio of Compound A-HCl, or solvate thereof, to the dispersion polymer is about 1:5. In some embodiments, the weight ratio of Compound A-HCl, or solvate thereof, to the dispersion polymer is about 1:4. In some embodiments, the weight ratio of Compound A-HCl, or solvate thereof, to the dispersion polymer is about 1:3. In some embodiments, the weight ratio of Compound A-HCl, or solvate thereof, to the dispersion polymer is about 1:2. In some embodiments, the weight ratio of Compound A-HCl, or solvate thereof, to the dispersion polymer is about 1:1.

In some embodiments, the spray-dried solid dispersion comprises at least 5% by weight of Compound A-HCl, or solvate thereof. In some embodiments, the spray-dried solid dispersion comprises at least 10% by weight of Compound A-HCl, or solvate thereof. In some embodiments, the spray-dried solid dispersion comprises at least 15% by weight of Compound A-HCl, or solvate thereof. In some embodiments, the spray-dried solid dispersion comprises at least 20% by weight of Compound A-HCl, or solvate thereof. In some embodiments, the spray-dried solid dispersion comprises at least 25% by weight of Compound A-HCl, or solvate thereof. The % amounts are calculated based on the free base, i.e., Compound A.

In some embodiments, the spray-dried solid dispersion comprises about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25% by weight of Compound A-HCl, or solvate thereof. In some embodiments, the spray-dried solid dispersion comprises about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, or about 35% by weight of Compound A-HCl, or solvate thereof. In some embodiments, the spray-dried solid dispersion comprises about 15% of Compound A-HCl, or solvate thereof. In some embodiments, the spray-dried solid dispersion comprises about 35% of Compound A-HCl, or solvate thereof. The % amounts are calculated based on the free base, i.e. Compound A.

In some embodiments, the spray-dried solid dispersion comprises about 5% by weight of Compound A-HCl, or solvate thereof. In some embodiments, the spray-dried solid dispersion comprises about 6% by weight of Compound A-HCl, or solvate thereof. In some embodiments, the spray-dried solid dispersion comprises about 7% by weight of Compound A-HCl, or solvate thereof. In some embodiments, the spray-dried solid dispersion comprises about 8% by weight of Compound A-HCl, or solvate thereof. In some embodiments, the spray-dried solid dispersion comprises about 9% by weight of Compound A-HCl, or solvate thereof. In some embodiments, the spray-dried solid dispersion comprises about 10% by weight of Compound A-HCl, or solvate thereof. In some embodiments, the spray-dried solid dispersion comprises about 11% by weight of Compound A-HCl, or solvate thereof. In some embodiments, the spray-dried solid dispersion comprises about 12% by weight of Compound A-HCl, or solvate thereof. In some embodiments, the spray-dried solid dispersion comprises about 13% by weight of Compound A-HCl, or solvate thereof. In some embodiments, the spray-dried solid dispersion comprises about 14% by weight of Compound A-HCl, or solvate thereof. In some embodiments, the spray-dried solid dispersion comprises about 15% by weight of Compound A-HCl, or solvate thereof. In some embodiments, the spray-dried solid dispersion comprises about 16% by weight of Compound A-HCl, or solvate thereof. In some embodiments, the spray-dried solid dispersion comprises about 17% by weight of Compound A-HCl, or solvate thereof. In some embodiments, the spray-dried solid dispersion comprises about 18% by weight of Compound A-HCl, or solvate thereof. In some embodiments, the spray-dried solid dispersion comprises about 19% by weight of Compound A-HCl, or solvate thereof. In some embodiments, the spray-dried solid dispersion comprises about 20% by weight of Compound A-HCl, or solvate thereof. In some embodiments, the spray-dried solid dispersion comprises about 21% by weight of Compound A-HCl, or solvate thereof. In some embodiments, the spray-dried solid dispersion comprises about 22% by weight of Compound A-HCl, or solvate thereof. In some embodiments, the spray-dried solid dispersion comprises about 23% by weight of Compound A-HCl, or solvate thereof. In some embodiments, the spray-dried solid dispersion comprises about 24% by weight of Compound A-HCl, or solvate thereof. In some embodiments, the spray-dried solid dispersion comprises about 25% by weight of Compound A-HCl, or solvate thereof. In some embodiments, the spray-dried solid dispersion comprises about 26% by weight of Compound A-HCl, or solvate thereof. In some embodiments, the spray-dried solid dispersion comprises about 27% by weight of Compound A-HCl, or solvate thereof. In some embodiments, the spray-dried solid dispersion comprises about 28% by weight of Compound A-HCl, or solvate thereof. In some embodiments, the spray-dried solid dispersion comprises about 29% by weight of Compound A-HCl, or solvate thereof. In some embodiments, the spray-dried solid dispersion comprises about 30% by weight of Compound A-HCl, or solvate thereof. In some embodiments, the spray-dried solid dispersion comprises about 31% by weight of Compound A-HCl, or solvate thereof. In some embodiments, the spray-dried solid dispersion comprises about 32% by weight of Compound A-HCl, or solvate thereof. In some embodiments, the spray-dried solid dispersion comprises about 33% by weight of Compound A-HCl, or solvate thereof. In some embodiments, the spray-dried solid dispersion comprises about 34% by weight of Compound A-HCl, or solvate thereof. In some embodiments, the spray-dried solid dispersion comprises about 35% by weight of Compound A-HCl, or solvate thereof. The % amounts are calculated based on the free base, i.e. Compound A.

In some embodiments, the spray-dried solid dispersion further comprises a non-aqueous solvent. In some embodiments, the non-aqueous solvent is present in detectable amounts. In some embodiments, the spray-dried solid dispersion is non-aqueous solvent free.

In some embodiments, the spray-dried solid dispersion further comprises a non-aqueous solvent selected from the group consisting of tert-butanol, n-propanol, n-butanol, isopropanol, ethanol, methanol, acetone, ethyl acetate, acetonitrile, methyl ethyl ketone, methyl isobutyl ketone, methyl acetate, and mixtures thereof. In some embodiments, the spray-dried solid dispersion further comprises a non-aqueous solvent selected from the group consisting of methanol, acetone, and mixtures thereof. In some embodiments, the spray-dried solid dispersion further comprises methanol.

### Tablets

In one aspect, described herein is a tablet comprising: Compound A-HCl, or solvate thereof, dispersed in a polymer matrix formed from a pharmaceutically acceptable polymer; one or more pharmaceutical acceptable ingredients selected from the group consisting of one or more diluents, one or more disintegrants, one or more lubricants, and one or more glidants; and optionally one or more film coating agents.

In some embodiments, Compound A-HCl, or solvate thereof, dispersed in a polymer matrix formed from a pharmaceutically acceptable polymer is the spray-dried solid dispersion described herein.

In some embodiments, tablets comprise about 2% by weight to about 20% by weight of Compound A-HCl, or solvate thereof. In some embodiments, tablets comprise about 2% by weight to about 15% by weight of Compound A-HCl, or solvate thereof.

In some embodiments, tablets comprise about 10% by weight to about 30% by weight of the polymer matrix formed from the pharmaceutically acceptable polymer. In some embodiments, tablets comprise about 20% by weight to about 35% by weight of the polymer matrix formed from the pharmaceutically acceptable polymer.

In some embodiments, tablets comprise about 2% by weight to about 10% by weight of Compound A-HCl, or solvate thereof, dispersed in about 10% by weight to about 30% by weight of a polymer matrix formed from a pharmaceutically acceptable polymer.

In some embodiments, tablets comprise about 2% by weight to about 10% by weight of Compound A-HCl, or solvate thereof, dispersed in about 10% by weight to about 30% by weight of a polymer matrix formed from a pharmaceutically acceptable polymer; about 40% by weight to about 80% by weight of one or more pharmaceutical acceptable ingredients selected from the group consisting of one or more diluents, one or more disintegrants, one or more lubricants, and one or more glidants; and optionally less than about 5% by weight of one or more film coating agents.

In some embodiments, in addition to the spray-dried solid dispersion, additional excipients in the tablets comprise one or more diluents, one or more disintegrants, one or more lubricants, one or more glidants, or any combination thereof. In some embodiments, in addition to the spray-dried solid dispersion, additional excipients in the tablets comprise microcrystalline cellulose, mannitol, crospovidone, colloidal silicon dioxide, and magnesium stearate.

In some embodiments, the tablet comprises one or more fillers/binders/diluents. Fillers/binders/diluents are selected from celluloses (such as microcrystalline cellulose, carboxymethylcellulose, ethyl cellulose and methyl cellulose), starch, gelatin, sugars (such as sucrose, glucose, dextrose, mannitol, and lactose), natural and synthetic gums (such as acacia, sodium alginate, panwar gum, and ghatti gum), polyvinylpyrrolidinone, polyethylene glycol, waxes, and any combinations thereof. In some embodiments, tablets comprise microcrystalline cellulose, and mannitol.

In some embodiments, the one or more fillers/binders/diluents in the tablets described herein comprise between about 20% and about 80% by weight of the total tablet weight. In some embodiments, the one or more fillers/binders/diluents in the tablets described herein comprise between about 40% and about 65% by weight of the total tablet weight. In some embodiments, the one or more fillers/binders/diluents in the tablets described herein comprise between about 50% and about 65% by weight of the total tablet weight. In some embodiments, the one or more fillers/binders/diluents in the tablets described herein comprise about 45%, about 50%, about 55%, about 60%, about 65%, or about 70% by weight of the total tablet weight. In some embodiments, the one or more fillers/binders/diluents in the tablets described herein comprise about 58% by weight of the total tablet weight. In some embodiments, less than 70% by weight, less than 65% by weight, less than 60% by weight, less than 55% by weight, or less than 50% by weight of the total tablet weight comprise one or more fillers/binders/diluents. In some embodiments, less than 60% by weight of the total tablet weight comprise one or more fillers/binders/diluents.

In some embodiments, tablets comprise one or more disintegrants. Disintegrants are selected from croscarmellose sodium, crospovidone, sodium starch glycolate, veegum HV, methylcellulose, agar, bentonite, cellulose, carboxymethyl cellulose, and any combination thereof. In some embodiments, tablets comprise crospovidone.

In some embodiments, the one or more disintegrants in the tablets described herein comprise between about 2% and about 30% by weight of the total tablet weight. In some embodiments, the one or more disintegrants in the tablets described herein comprise between about 5% and about 20% by weight of the total tablet weight. In some embodiments, the one or more disintegrants in the tablets described herein comprise between about 10% and about 20% by weight of the total tablet weight. In some embodiments, the one or more disintegrants in the tablets described herein comprise about 5%, about 6%, about 7%, about 8%, about 9%, about 10%, about 11%, about 12%, about 13%, about 14%, about 15%, about 16%, about 17%, about 18%, about 19%, or about 20% by weight of the total tablet weight. In some embodiments, the one or more disintegrants in the tablets described herein comprise about 15% by weight of the total tablet weight. In some embodiments, less than 20% by weight of the total tablet weight comprise one or more disintegrants.

In some embodiments, tablets comprise one or more lubricants. Lubricants are selected from talc, magnesium stearate, calcium stearate, stearic acid, sodium stearyl fumarate, glyceryl behenate, hydrogenated vegetable oils, polyethylene glycol, and any combinations thereof. In some embodiments, tablets comprise magnesium stearate.

In some embodiments, the one or more lubricants in the tablets described herein comprise between about 0.1% and about 5% by weight of the total tablet weight. In some embodiments, the one or more lubricants in the tablets described herein comprise between about 0.1% and about 2% by weight of the total tablet weight. In some embodiments, the one or more lubricants in the tablets described herein comprise between about 0.1% and about 1% by weight of the total tablet weight. In some embodiments, the one or more lubricants in the tablets described herein comprise about 0.1%, about 0.2%, about 0.3%, about 0.4%, about 0.5%, about 0.6%, about 0.7%, about 0.8%, about 0.9%, or about 1% by weight of the total tablet weight. In some embodiments, the one or more lubricants in the tablets described herein comprise about 0.5% by weight of the total tablet weight. In some embodiments, less than 2% by weight of the total tablet weight comprise one or more lubricants. In some embodiments, less than 1% by weight of the total tablet weight comprise one or more lubricants.

In some embodiments, tablets comprise one or more glidants. A glidant is a substance that is added to a powder to improve its flowability. Examples of glidants include magnesium stearate, colloidal silicon dioxide, starch and talc. In some embodiments, tablets comprise colloidal silicon dioxide.

In some embodiments, the one or more lubricants in the tablets described herein comprise between about 0.1% and about 5% by weight of the total tablet weight. In some embodiments, the one or more lubricants in the tablets described herein comprise between about 0.1% and about 2% by weight of the total tablet weight. In some embodiments, the one or more lubricants in the tablets described herein comprise between about 0.5% and about 1.5% by weight of the total tablet weight. In some embodiments, the one or more lubricants in the tablets described herein comprise about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, or about 2% by weight of the total tablet weight. In some embodiments, the one or more lubricants in the tablets described herein comprise about 1% by weight of the total tablet weight. In some embodiments, less than 2% by weight of the total tablet weight comprise one or more lubricants. In some embodiments, less than 1.5% by weight of the total tablet weight comprise one or more lubricants.

### Additional Excipients

In some embodiments, the tablet described herein comprises additional excipients including, but not limited, to buffering agents, glidants, preservatives, and coloring agents. Additional excipients such as bulking agents, tonicity agents, and chelating agents are within the scope of the embodiments.

Non-limiting examples of buffering agents include, but are not limited to, sodium bicarbonate, potassium bicarbonate, magnesium hydroxide, magnesium lactate, magnesium glucomate, aluminum hydroxide, aluminum hydroxide/sodium bicarbonate co precipitate, a mixture of an amino acid and a buffer, a mixture of aluminum glycinate and a buffer, a mixture of an acid salt of an amino acid and a buffer, and a mixture of an alkali salt of an amino acid and a buffer. Additional buffering agents include sodium citrate, sodium tartarate, sodium acetate, sodium carbonate, sodium polyphosphate, potassium polyphosphate, sodium pyrophosphate, potassium pyrophosphate, disodium hydrogenphosphate, dipotassium hydrogenphosphate, trisodium phosphate, tripotassium phosphate, sodium acetate, potassium metaphosphate, magnesium oxide, magnesium hydroxide, magnesium carbonate, magnesium silicate, calcium acetate, calcium glycerophosphate, calcium chloride, calcium hydroxide, calcium lactate, calcium carbonate, calcium bicarbonate, and other calcium salts.

In some embodiments, the tablet described herein comprises a preservative. Preservatives include anti-microbials, anti-oxidants, and agents that enhance sterility. Exemplary preservatives include ascorbic acid, ascorbyl palmitate, BHA, BHT, citric acid, erythorbic acid, fumaric acid, malic acid, propyl gallate, sodium ascorbate, sodium bisulfate, sodium metabisulfite, sodium sulfite, parabens (methyl-, ethyl-, butyl-), benzoic acid, potassium sorbate, vanillin, and the like.

In some embodiments, the tablet described herein comprises a coloring agent for identity and/or aesthetic purposes of the resultant liquid form. Suitable coloring agents illustratively include FD&C Red No. 3, FD&C Red No. 20, FD&C Red No. 40, FD&C Yellow No. 6, FD&C Blue No. 2, D&C Green No. 5, D&C Orange No. 5, caramel, ferric oxide and mixtures thereof.

Additional excipients are contemplated in the tablet embodiments. These additional excipients are selected based on function and compatibility with the tablet compositions described herein and may be found, for example in Remington: The Science and Practice of Pharmacy, Nineteenth Ed (Easton, PA: Mack Publishing Company, 1995); Hoover, John E., Remington's Pharmaceutical Sciences, (Easton, PA: Mack Publishing Co 1975); Liberman, H. A. and Lachman, L., Eds., Pharmaceutical Dosage Forms (New York, NY: Marcel Decker 1980); and Pharmaceutical Dosage Forms and Drug Delivery Systems, Seventh Ed (Lippincott Williams & Wilkins 1999), herein incorporated by reference in their entirety.

In further embodiments, the tablets described herein are coated tablets, such as enteric-coated tablets, sugar-coated, or film-coated tablets.

In one embodiment, the individual unit dosages also include film coatings, which disintegrate upon oral ingestion or upon contact with diluent. In one embodiment, these formulations are manufactured by conventional techniques.

Compressed tablets are solid dosage forms prepared by compacting the bulk blend formulations described above. In various embodiments, compressed tablets which are designed to dissolve in the mouth will include one or more flavoring agents. In other embodiments, the compressed tablets will include a film surrounding the final compressed tablet. In some embodiments, the film coating aids in patient compliance (e.g., Opadry® coatings or sugar coating). Film coatings comprising Opadry® typically range from about 1% to about 5% of the tablet weight. In other embodiments, the compressed tablets include one or more excipients.

Provided herein are film-coated tablets forms, which comprise: a combination of an active ingredient (e.g. Compound A-HCl) and one or more tableting excipients to form a tablet core and subsequently coating the core. The tablet cores are produced using conventional tableting processes and with subsequent compression and coating.

Enteric-coatings are coatings that resist the action of stomach acid but dissolve or disintegrate in the intestine.

In one aspect, the oral solid dosage form disclosed herein include an enteric coating(s). Enteric coatings include one or more of the following: cellulose acetate phthalate; methyl acrylate-methacrylic acid copolymers; cellulose acetate succinate; hydroxy propyl methyl cellulose phthalate; hydroxy propyl methyl cellulose acetate succinate (hypromellose acetate succinate); polyvinyl acetate phthalate (PVAP); methyl methacrylate-methacrylic acid copolymers; methacrylic acid copolymers, cellulose acetate (and its succinate and phthalate version); styrol maleic acid co-polymers; polymethacrylic acid/acrylic acid copolymer; hydroxyethyl ethyl cellulose phthalate; hydroxypropyl methyl cellulose acetate succinate; cellulose acetate tetrahydrophtalate; acrylic resin; shellac.

An enteric coating is a coating put on a tablet, pill, capsule, pellet, bead, granule, particle, etc. so that it doesn't dissolve until it reaches the small intestine.

Sugar-coated tablets are compressed tablets surrounded by a sugar coating, which may be beneficial in covering up objectionable tastes or odors and in protecting the tablets from oxidation.

Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets. In some embodiments, tablets are coated with water soluble, pH independent film coating which allows for immediate disintegration for fast, active release (e.g. Opadry products).

### Dosage in the Tablet

In some embodiments, the amount of Compound A-HCl, or solvate thereof, in the tablet is between about 5 mg and about 100 mg. In some embodiments, the amount of Compound A-HCl, or solvate thereof, in the tablet is between about 5 mg and about 80 mg. In some embodiments, the amount of Compound A-HCl, or solvate thereof, in the tablet is between about 5 mg and about 60 mg. In some embodiments, the amount of Compound A-HCl, or solvate thereof, in the tablet is between about 10 mg and about 40 mg.

In some embodiments, the amount of Compound A-HCl, or solvate thereof, in the tablet is about 10 mg. In some embodiments, the amount of Compound A-HCl, or solvate thereof, in the tablet is about 20 mg. In some embodiments, the amount of Compound A-HCl, or solvate thereof, in the tablet is about 30 mg. In some embodiments, the amount of Compound A-HCl, or solvate thereof, in the tablet is about 40 mg. In some embodiments, the amount of Compound A-HCl, or solvate thereof, in the tablet is about 50 mg. In some embodiments, the amount of Compound A-HCl, or solvate thereof, in the tablet is about 60 mg. In some embodiments, the amount of Compound A-HCl, or solvate thereof, in the tablet is about 70 mg. In some embodiments, the amount of Compound A-HCl, or solvate thereof, in the tablet is about 80 mg.

### Methods of Dosing and Treatment Regimens

In one embodiment, the pharmaceutical compositions disclosed herein are used as medicaments for the treatment of diseases or conditions in a mammal that would benefit from modulation of somatostatin activity. Methods for treating any of the diseases or conditions described herein in a mammal in need of such treatment, involves administration of pharmaceutical compositions that include Compound A, or a pharmaceutically acceptable salt thereof, in therapeutically effective amounts to said mammal.

In certain embodiments, the compositions containing Compound A described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In general, however, doses employed for adult human treatment are typically in the range of about 10 mg to about 100 mg per day of Compound A. In one embodiment, the desired dose is conveniently presented in a single dose or in divided doses administered simultaneously or at appropriate intervals, for example as two, three, four or more sub-doses per day.

In one aspect, Compound A, or a pharmaceutically acceptable salt thereof (e.g. Compound A-monohydrochloride), is administered daily to humans in need of therapy with a SST2 agonist. In some embodiments, Compound A, or a pharmaceutically acceptable salt thereof (e.g. Compound A-monohydrochloride), is administered once a day. In some embodiments, Compound A, or a pharmaceutically acceptable salt thereof (e.g. Compound A-monohydrochloride), is administered twice a day. In some embodiments, Compound A, or a pharmaceutically acceptable salt thereof (e.g. Compound A-monohydrochloride), is administered every other day.

In some embodiments, Compound A, or a pharmaceutically acceptable salt thereof (e.g. Compound A-monohydrochloride), is administered orally to the human on a continuous dosing schedule. In some embodiments, Compound A, or a pharmaceutically acceptable salt thereof (e.g. Compound A-monohydrochloride), is administered to the human on a continuous daily dosing schedule.

The term “continuous dosing schedule” refers to the administration of a particular therapeutic agent at regular intervals. In some embodiments, continuous dosing schedule refers to the administration of a particular therapeutic agent at regular intervals without any drug holidays from the particular therapeutic agent. In some other embodiments, continuous dosing schedule refers to the administration of a particular therapeutic agent in cycles. In some other embodiments, continuous dosing schedule refers to the administration of a particular therapeutic agent in cycles of drug administration followed by a drug holiday (for example, a wash out period or other such period of time when the drug is not administered) from the particular therapeutic agent. For example, in some embodiments the therapeutic agent is administered once a day, twice a day, three times a day, once a week, twice a week, three times a week, four times a week, five times a week, six times a week, seven times a week, every other day, every third day, every fourth day, daily for a week followed by a week of no administration of the therapeutic agent, daily for a two weeks followed by one or two weeks of no administration of the therapeutic agent, daily for three weeks followed by one, two or three weeks of no administration of the therapeutic agent, daily for four weeks followed by one, two, three or four weeks of no administration of the therapeutic agent, weekly administration of the therapeutic agent followed by a week of no administration of the therapeutic agent, or biweekly administration of the therapeutic agent followed by two weeks of no administration of the therapeutic agent. In some embodiments, daily administration is once a day. In some embodiments, daily administration is twice a day. In some embodiments, daily administration is three times a day.

The term “continuous daily dosing schedule” refers to the administration of a particular therapeutic agent every day at roughly the same time each day. In some embodiments, daily administration is once a day.

In certain embodiments wherein improvement in the status of the disease or condition in the human is not observed, the daily dose of a Compound A, or a pharmaceutically acceptable salt thereof (e.g. Compound A-monohydrochloride), is increased. In some embodiments, the frequency of administration is increased in order to provide repeat high Cₘₐₓ levels on a more regular basis (e.g. once-a-day dosing schedule is changed to a twice-a-day dosing schedule). In some embodiments, the frequency of administration is increased in order to provide maintained or more regular exposure to Compound A, or a pharmaceutically acceptable salt thereof (e.g. Compound A-monohydrochloride). In some embodiments, the frequency of administration is increased in order to provide repeat high Cₘₐₓ levels on a more regular basis and provide maintained or more regular exposure to Compound A, or a pharmaceutically acceptable salt thereof (e.g. Compound A-monohydrochloride).

Generally, a suitable dose of Compound A, or a pharmaceutically acceptable salt thereof (e.g. Compound A-monohydrochloride) for administration to a human will be in the range of from about 0.01 mg/day to about 100 mg/day; from about 10 mg/day to about 100 mg/day; or from about 10 mg/day to about 80 mg/day. In other embodiments, a suitable dose of Compound A, or a pharmaceutically acceptable salt thereof (e.g. Compound A-monohydrochloride) for administration to a human will be about 10 mg/day, about 20 mg/day, about 30 mg/day, about 40 mg/day, about 50 mg/day, about 60 mg/day, about 70 mg/day, about 80 mg/day, about 90 mg/day, or about 100 mg/day. In some embodiments, a suitable dose of Compound A, or a pharmaceutically acceptable salt thereof (e.g. Compound A-monohydrochloride) for administration to a human will be about 10 mg/day. In some embodiments, a suitable dose of Compound A, or a pharmaceutically acceptable salt thereof (e.g. Compound A-monohydrochloride) for administration to a human will be about 20 mg/day. In some embodiments, a suitable dose of Compound A, or a pharmaceutically acceptable salt thereof (e.g. Compound A-monohydrochloride) for administration to a human will be about 30 mg/day. In other embodiments, a suitable dose of Compound A, or a pharmaceutically acceptable salt thereof (e.g. Compound A-monohydrochloride) for administration to a human will be about 40 mg/day. In other embodiments, a suitable dose of Compound A, or a pharmaceutically acceptable salt thereof (e.g. Compound A-monohydrochloride) for administration to a human will be about 50 mg/day. In other embodiments, a suitable dose of Compound A, or a pharmaceutically acceptable salt thereof (e.g. Compound A-monohydrochloride) for administration to a human will be about 60 mg/day. In other embodiments, a suitable dose of Compound A, or a pharmaceutically acceptable salt thereof (e.g. Compound A-monohydrochloride) for administration to a human will be about 70 mg/day. In other embodiments, a suitable dose of Compound A, or a pharmaceutically acceptable salt thereof (e.g. Compound A-monohydrochloride) for administration to a human will be about 80 mg/day. In some embodiments, dosages are administered once per day. In some embodiments, the amounts referenced above refer to the amount of Compound A-monohydrochloride.

In some embodiments, Compound A is administered at least 30 minutes before a meal. In some embodiments, Compound A is administered at least 60 minutes before a meal. In some embodiments, Compound A is administered at least 180 minutes after a meal. In some embodiments, Compound A is administered at least 60 minutes before a meal and at least 180 minutes after a meal. In some embodiments, Compound A is administered on an empty stomach. In some embodiments, Compound A is administered with a glass of water on an empty stomach at least 30 minutes before a meal. In some embodiments, Compound A is administered before bedtime. In some embodiments, Compound A is administered in a dosage regimen that comprises one or more of the aforementioned steps of administration.

In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the disease or condition to be treated, the mode of administration, the requirements of the subject, the severity of the disease or condition being treated, the identity (e.g., weight) of the human, and the particular additional therapeutic agents that are administered (if applicable), and the judgment of the practitioner.

### Treatment Based on Biomarker Detection

Both the GH and IGF-1 plasma concentrations are typically increased in active acromegaly and will decrease during effective treatment. Both biomarkers are therefore used in clinical practice to monitor biochemical control in acromegaly and to determine treatment effectiveness. As a result of the biological mechanisms underlying acromegaly, the most recent guidelines, by the Endocrine Society and the American Association of Clinical Endocrinologists (AACE), for the diagnosis and treatment monitoring in acromegaly focus on three key biomarkers; (a) IGF-1, (b) (mean) GH and (c) level of suppression of GH concentrations during an oral glucose tolerance test (OGTT).

In clinical practice, a main treatment goal is a reduction in insulin-like growth factor-1 (IGF-1) concentrations to the clinically accepted ‘normal’ values for age and sex, which has been associated with improved/normalized mortality. In the guidelines, the upper limit of normal (ULN) was introduced as a surrogate for ‘safe’ IGF-1 levels which can be used to monitor the biochemical control of an individual acromegaly patient. This ULN is commonly defined by 2× the standard deviation (SD) of normal values, for age and sex, where age related changes have the largest impact on IGF-1 concentrations. Additionally, ULN corrected values have the added benefit that it can be used as a comparable measure of IGF-1 concentrations between individuals.

The primarily pathologically affected hormone in acromegaly is growth hormone (GH). A random GH measurement is therefore performed to provide an indication of the actual endogenous 24 h GH profile. However, the use of random GH levels, or the mean of multiple samples, to monitor treatment effectiveness has many challenges (e.g. highly pulsatile profile of GH, assay variability, lack of a safe range) but requires minimal clinical effort to obtain, compared to a full 24 h GH profile with short sampling intervals which is not feasible in clinical practice. Therefore, IGF-1 is generally considered as a better and more stable biomarker.

Clinical manifestations due to excess GH and IGF-I concentrations include cardiovascular, cerebrovascular, and respiratory diseases and metabolic abnormalities. GH opposes the effects of insulin on carbohydrate metabolism, and impaired glucose tolerance and diabetes mellitus are frequent complications reported in up to 50% of patients with acromegaly. It is well documented that lowering GH levels and normalization of IGF-I concentrations in patients with acromegaly improves glucose homeostasis. Somatostatin analogs alter glucose homeostasis by inhibiting GH and insulin secretion. Pegvisomant is a specific GH receptor antagonist that does not directly affect insulin secretion. In two pilot studies (one in normal volunteers and one in patients with acromegaly), glucose homeostasis tended to deteriorate with octreotide treatment (Parkinson C, et al., J. Clin. Endocrinol. Metab. 87:1797-1804, 2002; Quabbe H J, et al, J. Clin. Endocrinol. Metab. 68:873-881, 1989), whereas treatment with pegvisomant had a neutral or positive effect. In patients with acromegaly, the presence of diabetes has been observed to contribute to premature mortality, and impaired glucose tolerance has been found to correlate with the severity of acromegalic cardiomyopathy.

An oral glucose tolerance test (OGTT) is performed as a test to differentiate between healthy individuals and patients with active acromegaly. Furthermore, an OGTT can be performed already 1 week after surgery to assess successful reduction of GH secretion. In healthy individuals, the increase in plasma glucose levels suppresses GH secretion to well below 1 ng/ml. Insufficient suppression of GH is indicative for disruption in the regulation of the hypothalamus-pituitary-somatotropic axis. A ‘standard’ OGTT is performed using 75 g of orally administered glucose and the monitoring of blood samples for GH concentrations every 30 min for 2 h.

In a previous Phase 1 clinical trial evaluating repeated daily administration of Compound A-HCl capsules in healthy volunteers over 7 days (5 mg) and 10 days (10 mg, 20 mg, and 30 mg), two treatment emergent adverse effects (TEAEs) of asymptomatic hyperglycemia occurred. Both events occurred 1 hour post oral glucose and resolved by 2 hours post load during the scheduled OGTT following the completion of 10 days of Compound A dosing. One subject that received the 20 mg doses experienced a peak glucose concentration of 12.2 mmol/L (220 mg/dL) 1 hour post oral glucose challenge which normalized to 5.8 mmol/L (104 mg/dL) by 2 hours post challenge. One subject that received the 10 mg doses experienced a peak glucose concentration of 12.4 mmol/L (223 mg/dL) 1 hour post oral glucose challenge that normalized to 4.3 mmol/L (77 mg/dL) by 2 hours post-challenge. No symptoms of hyperglycemia or other sequelae were reported in these cases. Compared to baseline, there was a small but significant incease in glucose AUC_{0-3 hr} upon 75 g oral glucose challenge at the highest multiple dose of 30 mg (3.2 h·mmol/L; 95% CI 1.8-4.6) compared to a smaller and not significant change in placebo (1.5 h·mmol/L; 95% CI—1.5-4.6). Fasting plasma glucose wasn't significantly changed.

In some embodiments, hyperglycemia comprises peak serum glucose concentrations >100 mg/dL, >110 mg/dL, >120 mg/dL, >130 mg/dL, >140 mg/dL, >150 mg/dL, >160 mg/dL, >170 mg/dL, >180 mg/dL, >190 mg/dL, >200 mg/dL, >210 mg/dL, >220 mg/dL, >230 mg/dL, >240 mg/dL, or >250 mg/dL. In some embodiments, hyperglycemia comprises peak serum glucose concentrations >100 mg/dL, >110 mg/dL, >120 mg/dL, >130 mg/dL, >140 mg/dL, >150 mg/dL, >160 mg/dL, >170 mg/dL, >180 mg/dL, >190 mg/dL, or >200 mg/dL. In some embodiments, hyperglycemia comprises peak serum glucose concentrations >100 mg/dL, >110 mg/dL, >120 mg/dL, >130 mg/dL, >140 mg/dL, >150 mg/dL, >160 mg/dL, >170 mg/dL, >180 mg/dL, >190 mg/dL, or >200 mg/dL, as measured by OGTT.

Modest postprandial hyperglycemia demonstrated via oral glucose tolerance, without changes in fasting glucose or HbA1c, has also been previously described with this pharmacologic class (Mazziotti G, et al., (2009) J. Clin. Endocrinol. Metab. 94(5):1500-1508). This is in contrast to the less selective pasireotide which has potent activity for SST5 receptor and is causally associated with decrease in insulin secretion and hyperglycemia (Henry R R, et al., (2013) J. Clin. Endocrinol. Metab. 98(8):3446-3453).

In some embodiments, hypoglycemia or hyperglycemia may occur during treatment with Compound A. In some embodiments, hypoglycemia may occur during treatment with Compound A. In some embodiments, hyperglycemia may occur during treatment with Compound A. In some embodiments, glucose monitoring is recommended, and antidiabetic treatment may need adjustment or need to be initiated when Compound A is administered as described herein.

In some embodiments, blood glucose levels are monitored when treatment with Compound A is initiated, or when the dose of Compound A is altered. Anti-diabetic treatment should be initiated or adjusted accordingly.

In some embodiments, blood glucose levels are monitored when treatment with Compound A is initiated, or when the dose of Compound A is altered and anti-diabetic treatment is adjusted or initiated accordingly. In some embodiments, hyperglycemia is treated with insulin and/or insulin sensitizers. In some embodiments, hyperglycemia is treated with an agent used in the treatment of diabetes. In some embodiments, hyperglycemia is treated with: metformin, insulin, a sulfonylurea, a GLP-1 receptor agonist, a thiazolidinedione, glinide, a SGLT2 inhibitor, a DPP-4 inhibitor, an alpha-glucosidase inhibitor, pramlintide, or combinations thereof. In some embodiments, hyperglycemia is treated by adjusting the diet consumed by the subject.

In some embodiments, the administration of pharmaceutical compositions that include Compound A, or a pharmaceutically acceptable salt thereof (e.g. Compound A-monohydrochloride) is based on the patient's serum insulin-like growth factor-1 (IGF-1) value, serum growth hormone (GH) value, or both. In some embodiments, the administration of pharmaceutical compositions that include Compound A, or a pharmaceutically acceptable salt thereof (e.g. Compound A-monohydrochloride) is based on the patient's serum IGF-1 value. In some embodiments, the administration of pharmaceutical compositions that include Compound A, or a pharmaceutically acceptable salt thereof (e.g. Compound A-monohydrochloride) is based on the patient's serum GH value. In some embodiments, the compositions that include Compound A, or a pharmaceutically acceptable salt thereof (e.g. Compound A-monohydrochloride) are administered to patients with abnormal serum IGF-1 value, serum GH value, or both, for the treatment of any of the diseases or conditions described herein.

In some embodiments, the administration of pharmaceutical compositions that include Compound A, or a pharmaceutically acceptable salt thereof (e.g. Compound A-monohydrochloride) is based on achieving a serum IGF-1 value comparable to a serum IGF-1 value possible with LA-SRL therapy. For many patients, parenteral administration of LA-SRL is painful and inconvenient and therapy with Compound A is desired. Such patients would prefer to switch from pain injection of a LA-SRL to oral administration with Compound A.

In clinical practice, a main treatment goal is a reduction in IGF-1 concentrations to the clinically accepted ‘normal’ values for age and sex, which has been associated with improved/normalized mortality. In the guidelines, the upper limit of normal (ULN) was introduced as a surrogate for ‘safe’ IGF-1 levels which can be used to monitor the biochemical control of an individual acromegaly patient. This ULN is commonly defined by 2× the standard deviation (SD) of normal values, for age and sex, where age related changes have the largest impact on IGF-1 concentrations. Additionally, ULN corrected values have the added benefit that it can be used as a comparable measure of IGF-1 concentrations between individuals.

In some embodiments, treatment with Compound A aims to achieve serum IGF-1 (×ULN) of less than about 2.5. In some embodiments, treatment with Compound A aims to achieve serum IGF-1 (×ULN) of less than about 2. In some embodiments, treatment with Compound A aims to achieve serum IGF-1 (×ULN) of less than about 1.8. In some embodiments, treatment with Compound A aims to achieve serum IGF-1 (×ULN) of less than about 1.5. In some embodiments, treatment with Compound A aims to achieve serum IGF-1 (×ULN) of less than about 1.3. In some embodiments, treatment with Compound A aims to achieve serum IGF-1 (×ULN) of less than about 1.2. In some embodiments, treatment with Compound A aims to achieve serum IGF-1 (×ULN) of greater than 0.5 and less than about 2.5. In some embodiments, treatment with Compound A aims to achieve serum IGF-1 (×ULN) of >1 and < about 2.5.

In some embodiments, Compound A, or a pharmaceutically acceptable salt thereof (e.g. Compound A-monohydrochloride) is administered via a titration schedule. In some embodiments, Compound A, or a pharmaceutically acceptable salt thereof (e.g. Compound A-monohydrochloride) is administered via a titration schedule to minimize adverse events associated with the administration of Compound A, or a pharmaceutically acceptable salt thereof (e.g. Compound A-monohydrochloride). In some embodiments, titration with Compound A, or a pharmaceutically acceptable salt thereof (e.g. Compound A-monohydrochloride), enables: a subject to tolerate Compound A, or a pharmaceutically acceptable salt thereof (e.g. Compound A-monohydrochloride); to minimize adverse events associated with the administration of Compound A, or a pharmaceutically acceptable salt thereof (e.g. Compound A-monohydrochloride); maximizes the likelihood that an optimized dose of Compound A, or a pharmaceutically acceptable salt thereof (e.g. Compound A-monohydrochloride), will be administered to the subject and tolerated; or a combination thereof. In some embodiments, titration comprises up-titration.

In some embodiments, treatment with Compound A, or a pharmaceutically acceptable salt thereof (e.g. Compound A-monohydrochloride) does not require titration.

As used herein, a subject is said to “tolerate” a dose of a compound if administration of that dose to that subject does not result in an unacceptable adverse event or an unacceptable combination of adverse events. One of skill in the art will appreciate that tolerance is a subjective measure and that what may be tolerable to one patient may not be tolerable to a different patient. For example, one subject may not be able to tolerate headache, whereas a second subject may find mild headache tolerable but is not able to tolerate moderate headache, whereas a third subject is able to tolerate moderate headache but not severe headache. In some embodiments, a patient cannot tolerate treatment with an injectable somatostatin analog due to injection site rejections. In some embodiments, a patient cannot tolerate treatment with an orally adminstered somatostatin analog due to the need to consume multiple dose of the somatostatin analog more than once per day. In some embodiments, a patient cannot tolerate treatment with an orally adminstered somatostatin analog due to contraindications, such as hypersensitivity to octreotide or anaphylactoid reactions, including anaphylactic shock, which have been reported in patients receiving octreotide.

As used herein, an “adverse event” is an untoward medical occurrence that is associated with treatment with Compound A, or a pharmaceutically acceptable salt thereof. In some embodiments, an adverse event is headache, fatigue, diarrhea, pain, or combination thereof.

As used herein, an “optimized dose” refers a therapeutic dose optimized to the needs of a specific subject and is the highest dose of Compound A, or a pharmaceutically acceptable salt thereof (e.g. Compound A-monohydrochloride) that is equivalent to the needed dose of Compound A, that elicits the biological or medicinal response in the subject that is being sought and that can be tolerated by the subject, as determined by the subject, optionally in consultation with the subject's healthcare practitioner.

As used herein, “up-titration” of a compound refers to increasing the amount of a compound until the subject does not tolerate the increased amount. Up-titration can be achieved in one or more dose increments, which may be the same or different. In some embodiments, the method comprises administering Compound A, or a pharmaceutically acceptable salt thereof (e.g. Compound A-monohydrochloride), at an initial dose once daily for an initial period of time followed by up-titration to a higher dose of Compound A, or a pharmaceutically acceptable salt thereof (e.g. Compound A-monohydrochloride), once daily thereafter. In some embodiments, the initial period of time comprises one day, about one week, about two weeks, about three weeks, about four weeks, about five weeks, about six weeks, about seven weeks, about eight weeks, about nine weeks, about ten weeks, about eleven weeks, or about twelve weeks. In some embodiments, this cycle is repeated until an optimized dose is achieved.

In some embodiments, the method of titration comprises administering Compound A, or a pharmaceutically acceptable salt thereof (e.g. Compound A-monohydrochloride), at an initial dose once daily for about one week, about two weeks, about three weeks, or about four weeks, followed by up-titration to a higher dose of Compound A, or a pharmaceutically acceptable salt thereof (e.g. Compound A-monohydrochloride), once daily thereafter. In some embodiments, this cycle is repeated until an optimized dose is achieved. In some embodiments, dose adjustments are made every week, every two weeks, every three weeks or every four weeks. In some embodiments, dose adjustments are made and/or repeated to achieve consistent IGF-1 suppression. In some embodiments, dose adjustments are made and/or repeated to achieve consistent IGF-1 suppression and IGF-1 (×ULN) of less than about 2.5.

In some embodiments, the method of titration comprises administering Compound A, or a pharmaceutically acceptable salt thereof (e.g. Compound A-HCl), at an initial dose once daily for about one week, about two weeks, about three weeks, about four weeks, about five weeks, about six weeks, about seven weeks, or about eight weeks, followed by up-titration to a higher dose of Compound A, or a pharmaceutically acceptable salt thereof (e.g. Compound A-HCl), once daily thereafter. In some embodiments, this cycle is repeated until an optimized dose is achieved. In some embodiments, the method comprises administering 10 mg of Compound A-HCl once daily for about one week, about two weeks, about three weeks, about four weeks, about five weeks, about six weeks, about seven weeks, or about eight weeks, followed by up-titration to about 20 mg of Compound A-HCl once daily thereafter.

In some embodiments, the method of titration comprises the up-titration, or down-titration followed by an optional re-up-titration of Compound A, or a pharmaceutically acceptable salt thereof (e.g. Compound A-HCl).

In some embodiments, the titration schedule comprises administering Compound A or a pharmaceutically acceptable salt thereof (e.g. Compound A-HCl), at an initial dose for about 1 week, 2 weeks, 3 weeks, or 4 weeks and, provided that the patient tolerates the initial dose, increasing the dose by an amount equal to an incremental value. In some cases, the incremental value for increasing the daily dose is equivalent to about 10 mg of Compound A-HCl.

In some embodiments, the initial dose is equivalent to about 10 mg to about 40 mg of Compound A-HCl. In some embodiments, the initial dose is equivalent to about 10 mg, about 20 mg, about 30 mg, or about 40 mg of Compound A-HCl. In some embodiments, the initial dose is equivalent to about 10 mg of Compound A-HCl. In some embodiments, the initial dose is equivalent to about 20 mg of Compound A-HCl.

In some embodiments, the titration schedule further comprises: administering Compound A, or a pharmaceutically acceptable salt thereof, at the increased dose for about one week, two weeks, three weeks, or four weeks and provided that the patient tolerates the increased dose, further increasing the dose by an incremental value. In some embodiments, the incremental value for increasing the daily dose is equivalent to about 10 mg/day of Compound A-monohydrochloride.

In some embodiments, the titration schedule is repeated until an optimized dose is obtained. An optimized dose provides efficacy of treatment while minimizes side effects with Compound A treatment. In some embodiments, treatment with Compound A, or a pharmaceutically acceptable salt thereof (e.g. Compound A-monohydrochloride) does not require titration. In some embodiments, an optimized dose is administered without titration.

In some embodiments, the optimized dose is equivalent to about 10 mg/day to about 80 mg/day of Compound A-monohydrochloride. In some embodiments, the optimized dose is equivalent to about 10 mg/day, about 20 mg/day, about 30 mg/day, about 40 mg/day, about 50 mg/day, about 60 mg/day, about 70 mg/day, or about 80 mg/day of Compound A-monohydrochloride. In some embodiments, the optimized dose is equivalent to about 30 mg/day, about 40 mg/day, about 50 mg/day, or about 60 mg/day of Compound A-monohydrochloride. In some embodiments, the optimized dose is equivalent to about 30 mg/day of Compound A-monohydrochloride. In some embodiments, the optimized dose is equivalent to about 40 mg/day of Compound A-monohydrochloride. In some embodiments, the optimized dose is equivalent to about 50 mg/day of Compound A-monohydrochloride. In some embodiments, the optimized dose is equivalent to about 60 mg/day of Compound A-monohydrochloride.

In phase 2 clinical trials of acromegaly patients previously treated with LA-SRLs, an evaluation of steady state IGF-1 changes as a function of Compound A-monohydrochloride dose showed that 10 and 20 mg per day resulted in IGF-1 levels that were above baseline, where baseline was the IGF-1 measured at the last LA-SRL injection. The switch to Compound A-monohydrochloride occurred 4 weeks after last LA-SRL injection. Doses of 30/day and 40 mg/day resulted in changes from baseline of near zero, indicating the 30/day and 40 mg/day doses were equally effective in suppressing IGF-1 as prior monotherapy with injected LA-SRLs.

In some embodiments, patients not achieving therapeutic targets with 40 mg/day of paltusotine monotherapy are administered an additional therapeutic agent. In some embodiments, achieving therapeutic targets with 40 mg/day of paltusotine monotherapy comprises IGF-1 levels at −1≤1×ULN. In some embodiments, achieving therapeutic targets with 40 mg/day of paltusotine monotherapy comprises IGF-1 levels lower than or about equal to prior injected LA-SRL monotherapy. In some embodiments, achieving therapeutic targets with 40 mg/day of paltusotine monotherapy comprises IGF-1 levels lower than prior injected LA-SRL monotherapy. In some embodiments, the combination therapy comprises Compound A-monohydrochloride and a dopamine agonist and/or growth hormone (GH) receptor antagonists. In some embodiments, the dopamine agonist is cabergoline, or a pharmaceutically acceptable salt thereof. In some embodiments, the GH receptor antagonist is pegvisomant. In some embodiments, the combination therapy comprises Compound A-monohydrochloride and cabergoline, or a pharmaceutically acceptable salt thereof. In some embodiments, cabergoline is administered at dose and frequency of about 0.5 mg/week to about 0.5 mg/day. In some embodiments, cabergoline is administered at a dose of about 0.5 mg/week, 0.5 mg every other day, 0.5 mg every third day, 0.5 mg every fourth day, 0.5 mg every fifth day, 0.5 mg every sixth day. In some embodiments, cabergoline is administered at a dose of about 0.5 mg/day.

A dose-response relationship was observed when evaluating the magnitude of the rise of IGF-1 during Compound A washout. Exposure-response modeling estimated the Compound A concentration at which 80% of maximal pharmacological response (EC80) is achieved. In some embodiments, administration of the SDD tablets described herein comprising about 30 mg/day to about 60 mg/day of Compound A-monohydrochloride provides daily trough concentrations that exceed EC80 and result in consistent IGF-1 suppression in patients with acromegaly.

In some embodiments, the trough blood plasma level of Compound A providing consistent IGF-1 suppression in patients with acromegaly is between about 20 ng/mL to about 150 ng/mL. In some embodiments, the trough blood plasma level of Compound A providing consistent IGF-1 suppression in patients with acromegaly is between about 25 ng/mL to about 80 ng/mL. In some embodiments, the trough blood plasma level of Compound A providing consistent IGF-1 suppression in patients with acromegaly is between about 30 ng/mL to about 80 ng/mL. In some embodiments, the trough blood plasma level of Compound A providing consistent IGF-1 suppression in patients with acromegaly is between about 30-60 ng/mL.

In some embodiments, the trough blood plasma level of Compound A providing consistent IGF-1 suppression in patients with acromegaly is greater than 10 ng/mL, greater than 15 ng/mL, greater than 20 ng/mL, greater than 25 ng/mL, greater than 30 ng/mL, greater than 35 ng/mL, greater than 40 ng/mL, greater than 45 ng/mL, greater than 50 ng/mL, greater than 55 ng/mL, greater than 60 ng/mL, greater than 65 ng/mL, greater than 70 ng/mL, greater than 75 ng/mL, or greater than 80 ng/mL.

In some embodiments, the trough blood plasma level of Compound A providing consistent IGF-1 suppression in patients with acromegaly is at least about 10 ng/mL, at least about 15 ng/mL, at least about 20 ng/mL, at least about 25 ng/mL, at least about 30 ng/mL, at least about 35 ng/mL, at least about 40 ng/mL, at least about 45 ng/mL, at least about 50 ng/mL, at least about 55 ng/mL, at least about 60 ng/mL, at least about 65 ng/mL, at least about 70 ng/mL, at least about 75 ng/mL, or at least about 80 ng/mL, at least about 85 ng/mL, at least about 90 ng/mL, at least about 95 ng/mL, or at least about 100 ng/mL.

In some embodiments, Compound A, or a pharmaceutically acceptable salt thereof (e.g. Compound A-monohydrochloride) is administered at doses sufficient to provide a trough concentration of Compound A that is about 20 ng/mL, about 21 ng/mL, about 22 ng/mL, about 23 ng/mL, about 24 ng/mL, about 25 ng/mL, about 26 ng/mL, about 27 ng/mL, about 28 ng/mL, about 29 ng/mL, about 30 ng/mL, about 31 ng/mL, about 32 ng/mL, about 33 ng/mL, about 34 ng/mL, about 35 ng/mL, about 36 ng/mL, about 37 ng/mL, about 38 ng/mL, about 39 ng/mL, about 40 ng/mL, about 41 ng/mL, about 42 ng/mL, about 43 ng/mL, about 44 ng/mL, about 45 ng/mL, about 46 ng/mL, about 47 ng/mL, about 48 ng/mL, about 49 ng/mL, about 50 ng/mL, about 51 ng/mL, about 52 ng/mL, about 53 ng/mL, about 54 ng/mL, about 55 ng/mL, about 56 ng/mL, about 57 ng/mL, about 58 ng/mL, about 59 ng/mL, about 60 ng/mL, about 61 ng/mL, about 62 ng/mL, about 63 ng/mL, about 64 ng/mL, about 65 ng/mL, about 66 ng/mL, about 67 ng/mL, about 68 ng/mL, about 69 ng/mL, about 70 ng/mL, about 71 ng/mL, about 72 ng/mL, about 73 ng/mL, about 74 ng/mL, about 75 ng/mL, about 76 ng/mL, about 77 ng/mL, about 78 ng/mL, about 79 ng/mL, about 80 ng/mL, about 81 ng/mL, about 82 ng/mL, about 83 ng/mL, about 84 ng/mL, about 85 ng/mL, about 86 ng/mL, about 87 ng/mL, about 88 ng/mL, about 89 ng/mL, about 90 ng/mL, about 91 ng/mL, about 92 ng/mL, about 93 ng/mL, about 94 ng/mL, about 95 ng/mL, about 96 ng/mL, about 97 ng/mL, about 98 ng/mL, about 99 ng/mL, about 100 ng/mL, or greater than about 100 ng/mL.

In some embodiments, Compound A, or a pharmaceutically acceptable salt thereof (e.g. Compound A-monohydrochloride) is administered at doses sufficient to provide a trough concentration of Compound A that is about 32 ng/mL. In some embodiments, Compound A, or a pharmaceutically acceptable salt thereof (e.g. Compound A-monohydrochloride) is administered at doses sufficient to provide a trough concentration of Compound A that of at least about 32 ng/mL. In some embodiments, Compound A, or a pharmaceutically acceptable salt thereof (e.g. Compound A-monohydrochloride) is administered at doses sufficient to provide a trough concentration of Compound A that is greater than about 32 ng/mL.

In some embodiments, once daily administration of the SDD tablets described herein at a total daily dose of about 20 mg to about 80 mg of Compound A-monohydrochloride provides a trough concentration of Compound A that is greater than 10 ng/mL, greater than 15 ng/mL, greater than 20 ng/mL, greater than 25 ng/mL, greater than 30 ng/mL, greater than 35 ng/mL, greater than 40 ng/mL, greater than 45 ng/mL, greater than 50 ng/mL, greater than 55 ng/mL, greater than 60 ng/mL, greater than 65 ng/mL, greater than 70 ng/mL, greater than 75 ng/mL, greater than 80 ng/mL, greater than 85 ng/mL, greater than 90 ng/mL, greater than 95 ng/mL, greater than 100 ng/mL, greater than 115 ng/mL, greater than 120 ng/mL, greater than 125 ng/mL, greater than 130 ng/mL, greater than 135 ng/mL, greater than 140 ng/mL, greater than 145 ng/mL, greater than 150 ng/mL, or greater than 150 ng/mL.

In some embodiments, once daily administration of the SDD tablets described herein at a total daily dose of about 20 mg to about 60 mg of Compound A-monohydrochloride provides a trough concentration of Compound A of about 10 ng/mL to about 150 ng/mL. In some embodiments, once daily administration of the SDD tablets described herein at a total daily dose of about 40 mg to about 60 mg of Compound A-monohydrochloride provides a trough concentration of Compound A of about 20 ng/mL to about 150 ng/mL. In some embodiments, once daily administration of the SDD tablets described herein at a total daily dose of about 40 mg of Compound A-monohydrochloride provides a trough concentration of Compound A of about 20 ng/mL to about 110 ng/mL. In some embodiments, once daily administration of the SDD tablets described herein at a total daily dose of about 40 mg of Compound A-monohydrochloride provides a trough concentration of Compound A of at least 30 ng/mL. In some embodiments, once daily administration of the SDD tablets described herein at a total daily dose of about 40 mg of Compound A-monohydrochloride provides a trough concentration of Compound A of at least 32 ng/mL. In some embodiments, once daily administration of the SDD tablets described herein at a total daily dose of about 40 mg of Compound A-monohydrochloride provides a trough concentration of Compound A that is about 20 ng/mL, about 21 ng/mL, about 22 ng/mL, about 23 ng/mL, about 24 ng/mL, about 25 ng/mL, about 26 ng/mL, about 27 ng/mL, about 28 ng/mL, about 29 ng/mL, about 30 ng/mL, about 31 ng/mL, about 32 ng/mL, about 33 ng/mL, about 34 ng/mL, about 35 ng/mL, about 36 ng/mL, about 37 ng/mL, about 38 ng/mL, about 39 ng/mL, about 40 ng/mL, about 41 ng/mL, about 42 ng/mL, about 43 ng/mL, about 44 ng/mL, about 45 ng/mL, about 46 ng/mL, about 47 ng/mL, about 48 ng/mL, about 49 ng/mL, about 50 ng/mL, about 51 ng/mL, about 52 ng/mL, about 53 ng/mL, about 54 ng/mL, about 55 ng/mL, about 56 ng/mL, about 57 ng/mL, about 58 ng/mL, about 59 ng/mL, about 60 ng/mL, about 61 ng/mL, about 62 ng/mL, about 63 ng/mL, about 64 ng/mL, about 65 ng/mL, about 66 ng/mL, about 67 ng/mL, about 68 ng/mL, about 69 ng/mL, about 70 ng/mL, about 71 ng/mL, about 72 ng/mL, about 73 ng/mL, about 74 ng/mL, about 75 ng/mL, about 76 ng/mL, about 77 ng/mL, about 78 ng/mL, about 79 ng/mL, or greater than about 80 ng/mL but less than 150 ng/mL.

In any of the aforementioned aspects are further embodiments comprising single administrations of the effective amount of the compound, including further embodiments in which the compound is administered once a day.

### EXAMPLES

The following examples are provided for illustrative purposes only and not to limit the scope of the claims provided herein.

### Example 1: Oral Capsules

Representative capsules are described below in Table 1.

TABLE 1

| | | Quantity per | % |
|---|---|---|---|
| Component Name | Function | Unit (mg) | w/w |
| Compound A-HCl^{a} | Drug substance | 10.00 | 5.00 |
| Microcrystalline Cellulose | Diluent | 85.5 | 42.77 |
| Mannitol | Diluent | 85.5 | 42.77 |
| Croscarmellose Sodium | Disintegrant | 7.4 | 3.70 |
| Vitamin E Polyethylene Glycol Succinate (TPGS) | Solubilizer | 8.5 | 4.27 |
| Colloidal Silicon Dioxide | Glidant | 2.0 | 1.00 |
| Sodium Stearyl Fumarate | Lubricant | 1.0 | 0.50 |
| Size 2 Gelatin Capsule^{c} | Capsule Shell | NA | NA |

| | | |
|---|---|---|
| Total | 200.00^{b} | 100.00 |

^{a}Amount corrected for assay and moisture, chloride and isopropyl alcohol content;
^{b}Capsule fill weight adjusted based upon blend assay;
^{c}Composed of red iron oxide, titanium dioxide and gelatin.

A representative description of the manufacturing process for the hot melt granulation capsules is as follows:

Stage 1: High Shear Wet Granulation: Melt the Vitamin E Polyethylene Glycol Succinate (TPGS). Compound A-HCl, mannitol, microcrystalline cellulose, croscarmellose sodium and silicon dioxide are charged into a high shear wet granulator and mixed. The melted Vitamin E TPGS is sprayed onto the granulation components.

Stage 2: Milling: The wet granulation is milled through a screening mill using an appropriately sized screen.

Stage 3: Blending: The sodium stearyl fumarate is sieved using an appropriately sized screen. The milled granulation is charged into the diffusion mixer (tumble) along with the sodium stearyl fumarate and blended.

Stage 4: Encapsulation: The 10 mg capsules were automatically encapsulated in Size 2 gelatin capsules.

### Example 2: Spray-Dried Solid Dispersions

Spray-dried solid dispersions were prepared with 15% by weight Compound A-HCl: 15/85 Compound A-HCl/HPMCAS-M and 15/85 Compound A-HCl/PVP VA64 formulations. The manufactures were completed using the BLD-150, the Bend Lab Dryer with 150 kg/hr drying gas capacity. The parameters varied were solution solids loading for the HPMCAS-M SDD formulation to help reduce nozzle bearding, and dryer outlet temperature for the PVP VA64 SDD to de-risk fluctuations that may occur during clinical manufacturing. The original process parameter screening plan specified manufacturing the decreased dryer outlet temperature condition with a larger orifice nozzle to produce larger particles, however based on the results of the first spray it was determined that the atomization pressure needed to achieve the desired solution flow rate would have been too low to fully atomize the solution with the larger orifice. As dryer outlet temperature tends to have more variability than solution flow rate, the parameter screen was shifted to focus on de-risking the dryer outlet temperature alone while ensuring fully atomized droplets. Dryer outlet temperature variation could affect residual solvent levels in the SDDs, which can affect physical and chemical stability. All sprays were completed successfully with good yields, and indicate a robust processing space for both formulations.

### 15/85 Compound A HPMCAS-M SDD Formulation Manufacturing Details

Three sub batches of 15/85 Compound A-HCl/HPMCAS-M SDDs were sprayed to explore the manufacture processing space and prepare for clinical trial manufacturing. A sub batch was sprayed first at 10 wt % solids, and significant nozzle bearding that appeared to affect the spray plume was observed after about 45 minutes on solution.

The solution was diluted to 8 wt %, and a sub batch was manufactured with a duration of 1 hour to ensure bearding was reduced. A very small amount of bearding was observed during this batch after about 50 minutes on solution, but did not appear to affect the atomization plume and the 8 wt % solids loading was selected.

Cooling water at 2 GPM and approximately 7° C. was run through the spray dryer lid throughout all sprays to keep the lid cool and prevent sticking and browning. No significant lid buildup or browning was seen throughout manufacturing. No cleaning was performed between sprays, and all sprays were completed from one solution with additional solvent added prior to manufacturing batches 2A and 2C. A summary of the manufacturing parameters used for all three sub batches is shown in Table 2.

TABLE 2
Manufacturing summary for 15/85 Compound A-HCl/HPMCAS-M SDDs.

| Parameter | Lot 2A | Lot 2B | Lot 2C |
|---|---|---|---|
| Bulk SDD collected (g) | 5316 | 2231 | 624 |
| Solids loading in solution (wt %) | 8 | 10 | 8 |
| Solvent | Methanol | | |
| Atomizer | Pressure swirl: Steinen A75 | | |
| Atomizing pressure (psig) | 370 | 301 | 345 |
| Solution flow (g/min) | 131 | 132 | 131 |
| Drying gas flow rate (g/min) | 1848 | 1848 | 1848 |
| Dryer inlet temperature (° C.) | 146 | 145 | 144 |
| Dryer outlet temperature (° C.) | 45 | 45 | 45 |
| Bulk secondary tray drying | 16 hours at 40° C./15% RH | 17 hours at 40° C./15% RH | |
| Dry yield (%)¹ | 96 | 96 | 93 |
| Spray duration (hours) | 8 | 3 | 1 |

| | |
|---|---|
| Calculated dryer relative saturation (%) | 11 |
| Calculated dew point (° C.) | 3 |

¹Dry yield is calculated as SDD samples and dry bulk collected divided by the amount of solids sprayed.

### 15/85 Compound A-HCl/PVP VA64 SDD Formulation Manufacturing Details

Process parameter screening sprays and an FPN demonstration batch were also completed for the 15/85 Compound A-HCl/PVP VA64 SDD formulation. The dryer outlet temperature was varied to de-risk process parameter variability to prepare for clinical trial manufacturing.

The process space was constrained by a maximum dryer outlet temperature, the dryer outlet temperature, and the minimum desired solution flow rate. A maximum inlet temperature of 160° C. was specified to avoid sticking or browning of SDD on the spray dryer lid, and the minimum flow rate was set at 100 g/min to ensure sufficient throughput. The minimum and maximum dryer outlet temperatures were chosen to be 40° C. and 65° C. respectively to ensure adequate particle drying and that the dryer outlet temperature will not be above the wet particle Tg.

The process parameter screening sprays for the PVP VA64 formulation explored the manufacturing space by varying the dryer outlet temperature. This allowed investigation of the effect of dryer relative saturation on particle residual solvent content, morphology, density, and stability.

Cooling water was run at 2 GPM and approximately 7° C. throughout all sprays to prevent lid buildup and browning, and neither were noted. No nozzle bearding was observed throughout all three manufactures. All sprays were completed from one solution. The manufacturing details of each sub batch are summarized in Table 3.

TABLE 3
15/85 Compound A-HCl*/PVP VA64 SDD process parameters

| Parameter | Lot 4A | Lot 4B | Lot 4C |
|---|---|---|---|
| Bulk SDD collected (g) | 5285 | 623 | 624 |
| Solids loading in solution (wt %) | 8 | | |
| Solvent | Methanol | | |
| Atomizer | Pressure swirl: Steinen A75 | | |
| Atomizing pressure (psig) | 422 | 449 | 445 |
| Solution flow (g/min) | 128 | 133 | 132 |
| Drying gas flow rate (g/min) | 1845 | 1848 | 1849 |
| Dryer inlet temperature (° C.) | 143 | 153 | 132 |
| Dryer outlet temperature (° C.) | 45 | 49 | 40 |
| Bulk secondary tray drying | 18 hours at 40° C./15% RH | 26 hours at 40° C./15% RH | |
| Dry yield (%)¹ | 92 | 86 | 91 |
| Spray duration (hours) | 8 | 1 | 1 |
| Calculated dryer relative saturation (%) | 11 | 9 | 14 |

| | |
|---|---|
| Calculated dew point (° C.) | 3 |

*Compound A-HCl, formulated on a basis of free base.
¹Dry yield is calculated as SDD samples and dry bulk collected divided by the amount of solids sprayed.

### SDD Characterization

Particle Properties: The particle size distribution and bulk and tapped densities were measured for each batch of Compound A-HCl SDD. The HPMCAS-M SDDs have larger particle sizes, which is expected because the HPMCAS-M solution is more viscous than the PVP VA64 solution which leads to larger droplets for a given nozzle configuration. The increased solids loading in solution of lot 2B led to larger particles than batches 2A and 2C, which is also due to the higher viscosity of the spray solution. The particle size distributions of all PVPVA-64 batches are similar, as expected.

The bulk and tapped densities of 2A and 2C are similar, while batch 2B has a slightly lower density potentially due to the larger particles. The bulk and tapped densities of all PVPVA-64 batches are similar, indicating a robust process with respect to the dryer outlet temperature effects on powder properties.

Residual Solvent and Water Content: The residual methanol and water of the six SDDs were measured using GC and KF respectively. All SDDs contained residual MeOH below ICH guideline of 0.3 wt % after secondary drying, suggesting adequate drying at 40° C./15% RH.

Morphology by SEM: The particle morphology of all six SDDs was evaluated via SEM. Each SDD showed typical morphology with no evidence of irregular particles suggesting adequate atomization for all conditions tested. The HPMCAS-M particles were primarily collapsed spheres while the PVP VA64 SDDs contain a larger fraction of spherical particles.

Crystallinity by PXRD: All six SDDs were evaluated for crystallinity using PXRD. All SDDs were amorphous by PXRD as evident in the absence of sharp diffraction peaks.

Thermal Characteristics by DSC: All six SDDs were characterized by modulated DSC. The results are tabulated in Table 4. The manufactured SDDs were all amorphous and homogenous by DSC as evident by the presence of a single glass transition in the reversing heat signal. Neither formulation shows signs of crystallization after the Tg suggesting low propensity for crystallization of Compound A at those temperatures for both formulations. Furthermore, both formulations showed high Tg relative to ambient temperatures suggesting low physical stability risks in dry conditions. Packaging to minimize humidity will be necessary for the PVP VA64 formulation.

TABLE 4
Tabulated thermal characteristics of the six batches as measured by DSC.

| | | Tg | | Delta Cp | |
|---|---|---|---|---|---|
| Sample | Lot | (° C.) | Std Dev* | (J/(g*° C.)) | Std Dev* |
| 15/85 Compound | 2A | 135.2 | 0.35 | 0.27 | 0.03 |
| A-HCl/HPMCAS-M | 2B | 134.7 | 0.36 | 0.25 | 0.02 |
| SDD | 2C* | 134.5 | 0.95 | 0.26 | 0.00 |
| 15/85 Compound | 4A* | 125.5 | 0.04 | 0.31 | 0.01 |
| A-HCl/PVP VA64 | 4B | 125.6 | 0.26 | 0.30 | 0.01 |
| SDD | 4C | 125.6 | 0.17 | 0.35 | 0.08 |

*Average is from n = 2 replicates. As a result, std deviation is actually calculated as range/2.

### SUMMARY

Physical stability observations: PVPVA64 SDD appeared to deliquesce upon storage with crystals observed at 3 months (40° C./75% RH open). Recommend storage with desiccant. HPMCAS-M SDD was physically stable through 6 months at 40° C./75% RH open.

Chemical Stability observations: Possible acid catalyzed degradation in the HPMCAS-M formulation on stability. Some degradation in the PVP VA64 formulation as well, but not as significant as the HPMCAS-M SDD. Packaging will be required for the PVPVA SDD which will be driven by physical stability concerns. The 15% w/w Compound A/PVP VA64 was selected as lead SDD formulation.

### 12-Month Stability: 15% Compound A-HCl/PVP-VA64 SDD

12-month SDD samples were held at 5° C., 25° C./60% RH, and 40° C./75% RH with desiccant. Samples for water analysis by Karl Fisher titration were prepared and analyzed immediately; the remaining samples were vacuum desiccated overnight to remove residual moisture and preserve the physical state of SDD's for further characterization. A list of the analytical tests performed for characterization included: appearance, water content by Karl Fisher titration, Powder x-ray diffraction (PXRD), scanning electron microscopy (SEM), thermal characterization by modulated differential scanning calorimetry (mDSC), dissolution performance by micro-centrifuge (MCT) test, assay and related substances by HPLC.

Conclusion from PXRD analysis on the 12-month SDD stability samples: There was no evidence of crystallinity in samples stored at 12 months at each of the stability conditions.

Conclusion from SEM analysis on the 12-month SDD stability samples: No particle fusion was observed across all stability conditions at 12 month. There was no evidence of crystallinity in samples stored at 12 months at each of the stability conditions.

Conclusion from SEM analysis on the 12-month SDD stability samples: No particle fusion is observed across all stability conditions at 12 months. There was no evidence of crystallinity in samples stored at 12 months at each of the stability conditions.

Conclusions from mDSC analysis on the 12-month SDD stability samples: Repeated analysis of the 12 month SDD sample held at 5° C. affords non-reproducible thermograms, the cause for this result is not known at this time. The 5° C. sample was determined to be physically stable by all other characterization techniques. The 12 month SDD samples held at 25° C./60% RH and 40° C./75% RH showed a single, reproducible Tg at 124-125° C., supporting the conclusion that the SDD is stable after 12 months of storage with desiccant at these conditions.

Conclusion from MCT dissolution analysis on the 12-month SDD stability samples: Non-sink dissolution performance of the 12 month stability samples is consistent with the initial (t₀) sample stored at −20° C.

### 35/65 Compound A-HCl/PVP VA64 SDD Formulation Manufacturing Details

Spray-dried solid dispersions were prepared with 35% by weight Compound A-HCl: 35/65 Compound A-HCl/PVP VA64 formulation. Manufacture of the SDD was completed using the SD-180 lab dryer. Secondary drying was completed using the Binder Convection Dryer. Manufacturing details are in Table 5. The spray was completed successfully with good yield.

TABLE 5
35/65 Compound A-HCl*/PVP VA64 SDD process parameters

| Parameter | Value |
|---|---|
| Bulk SDD collected (g) | 2796 |
| Solids loading in solution (wt %) | 10.8 |
| Solvent | Methanol |
| Atomizer | SD-90 with Pressure Swirl |
| Atomizing pressure (psig) | 320 |
| Solution flow (g/min) | 115 |
| Drying gas rate (acfm) | 80 |
| Dryer inlet temperature (° C.) | 100 |
| Dryer outlet temperature (° C.) | 45 |
| Condensore Setpoint (° C.) | −20 |
| Bulk secondary tray drying | 48 hours at 40° C./ambient pressure |

*Compound A-HCl, formulated on a basis of free base

### Example 3: Oral Tablets

Representative 10 mg, 20 mg, 30 mg, 40 mg, and 60 mg tablets are presented in Tables 6 to 13. Excipients used included: microcrystalline cellulose, mannitol, crospovidone, colloidal silicon dioxide, magnesium stearate, Opadry White 03K184116 (film coating).

TABLE 6
Representative 10 mg spray-dried dispersion tablets.

| | | | |
|---|---|---|---|
| Component Name | Function | Quantity per Unit (mg) | % w/w |

Spray-Dried Dispersion

| | | | |
|---|---|---|---|
| Compound A-HCl | Drug substance | 10.00 | 3.20 |
| PVP/VA 64 | Film Forming Agent | 56.67 | 18.15 |
| Methanol^{a} | Solvent | NA | NA |

Roller Compaction/Blending

| | | | |
|---|---|---|---|
| Microcrystalline Cellulose | Diluent | 130.31 | 41.74 |
| Mannitol | Diluent | 53.04 | 16.99 |
| Crospovidone | Disintegrant | 30.29 | 9.71 |
| Colloidal Silicon Dioxide | Glidant | 1.53 | 0.49 |
| Magnesium Stearate | Lubricant | 0.76 | 0.24 |

Blending/Compression

| | | | |
|---|---|---|---|
| Crospovidone | Disintegrant | 18.18 | 5.82 |
| Colloidal Silicon Dioxide | Glidant | 1.52 | 0.49 |
| Magnesium Stearate | Lubricant | 0.76 | 0.24 |

Film Coating

| | | | |
|---|---|---|---|
| Opadry ® White 03K18416^{b} | Film Coating Agent | 9.09 | 2.91 |
| Purified Water^{c} | Solvent | NA | NA |

| | | |
|---|---|---|
| Total | 312.15 | 100.0 |

^{a}Methanol removed on drying during the spray drying process.
^{b}Composed of Hypromellose 2910, titanium dioxide and triacetin.
^{c}Purified water removed on drying during the film coating process.

TABLE 7
Representative 20 mg spray-dried dispersion (HPMCAS-M) tablets.

| Formulation no. | A1 | A2 | A3 |
|---|---|---|---|
| Tablet Dose/Tablet Weight (mg/mg) | 20/400 | 20/400 | 20/400 |

| | | |
|---|---|---|
| Function | Ingredient | % w/w of Blend |

Pregranulation

| | | | | |
|---|---|---|---|---|
| Active | 15/85 Compound A-HCl/HPMCAS-M | 33.33% | 33.33% | 33.33% |
| Local PH Modifier | Citric Acid | — | — | 5.00% |
| Filler | Microcrystalline Cellulose (Avicel PH-102) | 38.11% | 40.11% | 36.78% |
| Filler | Mannitol (Mannogem EZ Spray Dried) | 19.06% | 20.06% | 18.39% |
| Disintegration Aid | Sodium Chloride (NaCl Powder) | — | — | — |
| Disintegrant | Sodium Starch Glycolate (Explotab) | 5.00% | — | — |
| Disintegrant | Crospovidone (PVP-XL) | — | 5.00% | 3.00% |
| Glidant | Silicon dioxide (Syloid 244 FP) | 0.50% | 0.50% | 0.50% |
| Lubricant | Magnesium Stearate | 0.25% | 0.25% | 0.25% |

Extragranular

| | | | | |
|---|---|---|---|---|
| Disintegrant | Sodium Starch Glycolate (Explotab) | 3.00% | — | — |
| Disintegrant | Crospovidone (PVP-XL) | — | — | 2.00% |
| Glidant | Silicon dioxide (Syloid 244 FP) | 0.50% | 0.50% | 0.50% |
| Lubricant | Magnesium Stearate | 0.25% | 0.25% | 0.25% |

TABLE 8
Representative 20 mg spray-dried dispersion (PVPVA 64) tablets.

| Formulation no. | B1 | B2 | B3 |
|---|---|---|---|
| Tablet Dose/Tablet Weight (mg/mg) | 20/400 | 20/400 | 20/400 |

| | | |
|---|---|---|
| Function | Ingredient | % w/w of Blend |

Pregranulation

| | | | | |
|---|---|---|---|---|
| Active | 15/85 Compound A-HCl/PVPVA 64 | 33.33% | 33.33% | 33.33% |
| Local PH Modifier | Citric Acid | 5.00% | — | — |
| Filler | Microcrystalline Cellulose (Avicel PH-102) | 43.17% | 33.17% | 33.17% |
| Filler | Mannitol (Mannogem EZ Spray Dried) | — | 20.00% | — |
| Disintegration Aid | Sodium Chloride (NaCl Powder) | 5.00% | — | 20.00% |
| Disintegrant | Sodium Starch Glycolate (Explotab) | 6.00% | — | — |
| Disintegrant | Crospovidone (PVP-XL) | — | 6.00% | 6.00% |
| Glidant | Silicon dioxide (Syloid 244 FP) | 0.50% | 0.50% | 0.50% |
| Lubricant | Magnesium Stearate | 0.25% | 0.25% | 0.25% |

Extragranular

| | | | | |
|---|---|---|---|---|
| Disintegrant | Sodium Starch Glycolate (Explotab) | 6.00% | — | — |
| Disintegrant | Crospovidone (PVP-XL) | — | 6.00% | 6.00% |
| Glidant | Silicon dioxide (Syloid 244 FP) | 0.50% | 0.50% | 0.50% |
| Lubricant | Magnesium Stearate | 0.25% | 0.25% | 0.25% |

TABLE 9
Additional representative 20 mg spray dried dispersion tablets.

| Formulation no. | A4 | B4 | B5 | B6 |
|---|---|---|---|---|
| Tablet Dose/Tablet Weight (mg/mg) | 20/400 | 20/606.1 | 20/400 | 20/606.1 |

| | | |
|---|---|---|
| Function | Ingredient | % w/w of Blend |

Pregranulation

| | | | | | |
|---|---|---|---|---|---|
| Active | 15/85 Compound A-HCl/HPMCAS-M | 33.33% | — | — | — |
| Active | 15/85 Compound A-HCl/PVPVA 64 | — | 22.00% | 33.33% | 22.00% |
| Local PH | Citric Acid | 5.00% | 5.00% | — | — |
| Modifier | | | | | |
| Filler | Microcrystalline Cellulose (Avicel PH-102) | 35.61% | 40.50% | 33.17% | 43.00% |
| Filler | Mannitol (Mannogem EZ Spray Dried) | 16.56% | 15.00% | 16.00% | 17.50% |
| Disintegrant | Crospovidone (PVP-XL) | 5.00% | 10.00% | 10.00% | 10.00% |
| Glidant | Silicon dioxide (Syloid 244 FP) | 0.50% | 0.50% | 0.50% | 0.50% |
| Lubricant | Magnesium Stearate | 0.25% | 0.25% | 0.25% | 0.25% |

Extragranular

| | | | | | |
|---|---|---|---|---|---|
| Disintegrant | Crospovidone (PVP-XL) | 3.00% | 6.00% | 6.00% | 6.00% |
| Glidant | Silicon dioxide (Syloid 244 FP) | 0.50% | 0.50% | 0.50% | 0.50% |
| Lubricant | Magnesium Stearate | 0.25% | 0.25% | 0.25% | 0.25% |

TABLE 10
Representative 30 mg and 40 mg spray-dried
dispersion (PVPVA 64) tablets.

| Formulation no. | | D1 | D2 |
|---|---|---|---|
| Tablet Dose/Tablet Weight (mg/mg) | | 30/909.2 | 40/1212.3 |
| Function | Ingredient | % w/w of Blend | |
| Active | 15/85 Compound A-HCl/ | 22.00 | 22.00 |
| | PVPVA 64 | | |
| Filler | Microcrystalline Cellulose | 43.00 | 43.00 |
| | (Avicel PH-101) | | |
| Filler | Mannitol (Parteck M100) | 17.50 | 17.50 |
| Disintegrant | Crospovidone (PVP-XL) | 10.00 | 10.00 |
| Glidant | Silicon dioxide (Syloid 244 FP) | 0.50 | 0.50 |
| Lubricant | Magnesium Stearate | 0.25 | 0.25 |
| Disintegrant | Crospovidone (PVP-XL) | 6.00 | 6.00 |
| Glidant | Silicon dioxide (Syloid 244 FP) | 0.50 | 0.50 |
| Lubricant | Magnesium Stearate | 0.25 | 0.25 |

TABLE 11
Additional representative 40 mg spray-dried dispersion (PVPVA 64) tablets.

| Formulation no. | E1 | E2 | E3 |
|---|---|---|---|
| Tablet Dose/Tablet Weight (mg/mg) | 40/500 | 40/600 | 40/600 |

| | | |
|---|---|---|
| Function | Ingredient | % w/w of Blend |

Pregranulation

| | | | | |
|---|---|---|---|---|
| Active | 35/65 Compound A-HCl/PVPVA 64 | 34.29% | 28.57% | 28.57% |
| Filler | Microcrystalline Cellulose (Avicel PH-101) | 32.14% | 28.18% | 22.18% |
| Filler | Mannitol (Parteck M100) | 16.07% | 14.09% | 11.09% |
| Disintegrant | Croscarmellose Sodium (Ac-Di-Sol) | 5.00% | 5.00% | 7.81% |
| Disintegrant | Crospovidone (PVP-XL) | 5.00% | 5.00% | 7.81% |
| Glidant | Silicon dioxide (Syloid 244 FP) | 0.50% | 0.50% | 0.50% |
| Lubricant | Magnesium Stearate | 0.25% | 0.25% | 0.25% |

Extragranular

| | | | | |
|---|---|---|---|---|
| Filler | Microcrystalline Cellulose (Avicel PH-101) | — | 11.67% | 11.67% |
| Disintegrant | Croscarmellose Sodium (Ac-Di-Sol) | 3.00% | 3.00% | 4.69% |
| Disintegrant | Crospovidone (PVP-XL) | 3.00% | 3.00% | 4.69% |
| Glidant | Silicon dioxide (Syloid 244 FP) | 0.50% | 0.50% | 0.50% |
| Lubricant | Magnesium Stearate | 0.25% | 0.25% | 0.25% |

TABLE 12
Representative 60 mg spray-dried dispersion (PVPVA 64) tablets.

| Formulation no. | | |
|---|---|---|
| C1 | C2 | C3 |
| Tablet Dose/Tablet Weight | | |
| (mg/mg) | | |
| 60/500 | 60/600 | 60/600 |

| | | |
|---|---|---|
| Function | Ingredient | % w/w of Blend |

Pregranulation

| | | | | |
|---|---|---|---|---|
| Active | 35/65 Compound A-HCl/PVPVA 64 | 34.29% | 28.57% | 28.57% |
| Filler | Microcrystalline Cellulose (Avicel PH-101) | 32.14% | 28.18% | 22.18% |
| Filler | Mannitol (Parteck M100) | 16.07% | 14.09% | 11.09% |
| Disintegrant | Croscarmellose Sodium (Ac-Di-Sol) | 5.00% | 5.00% | 7.81% |
| Disintegrant | Crospovidone (PVP-XL) | 5.00% | 5.00% | 7.81% |
| Glidant | Silicon dioxide (Syloid 244 FP) | 0.50% | 0.50% | 0.50% |
| Lubricant | Magnesium Stearate | 0.25% | 0.25% | 0.25% |

Extragranular

| | | | | |
|---|---|---|---|---|
| Filler | Microcrystalline Cellulose (Avicel PH-101) | — | 11.67% | 11.67% |
| Disintegrant | Croscarmellose Sodium (Ac-Di-Sol) | 3.00% | 3.00% | 4.69% |
| Disintegrant | Crospovidone (PVP-XL) | 3.00% | 3.00% | 4.69% |
| Glidant | Silicon dioxide (Syloid 244 FP) | 0.50% | 0.50% | 0.50% |
| Lubricant | Magnesium Stearate | 0.25% | 0.25% | 0.25% |

TABLE 13
Additional representative 60 mg spray-dried dispersion (PVPVA 64) tablets.

| Formulation no. | | |
|---|---|---|
| C4 | C5 | C6 |
| Tablet Dose/Tablet Weight | | |
| (mg/mg) | | |
| 60/600 | 60/600 | 60/600 |

| | | |
|---|---|---|
| Function | Ingredient | % w/w of Blend |

Pregranulation

| | | | | |
|---|---|---|---|---|
| Active | 35/65 Compound A-HCl/PVPVA 64 | 28.57% | 28.57% | 28.57% |
| Filler | Microcrystalline Cellulose (Avicel PH-101) | 15.08% | 18.41% | 18.41% |
| Filler | Mannitol (Parteck M100) | 7.54% | 9.21% | 9.21% |
| Disintegrant | Pregelatinized Starch (Starch 1500) | 20.00% | — | — |
| Disintegration Aid | Sodium Chloride (NaCl Powder) | — | 10.00% | — |
| Disintegration Aid | 1:1 Sodium Chloride:Potassiinn Chloride | — | — | 10.00% |
| Disintegrant | Croscarmellose Sodium (Ac-Di-Sol) | 5.00% | 5.00% | 5.00% |
| Disintegrant | Crospovidone (PVP-XL) | 5.00% | 5.00% | 5.00% |
| Glidant | Silicon dioxide (Syloid 244 FP) | 0.50% | 0.50% | 0.50% |
| Lubricant | Magnesium Stearate | 0.25% | 0.25% | 0.25% |

Extragranular

| | | | | |
|---|---|---|---|---|
| Filler | Microcrystalline Cellulose (Avicel PH-101) | 11.31% | 11.31% | 11.31% |
| Disintegrant | Croscarmellose Sodium (Ac-Di-Sol) | 3.00% | 3.00% | 3.00% |
| Disintegrant | Crospovidone (PVP-XL) | 3.00% | 3.00% | 3.00% |
| Glidant | Silicon dioxide (Syloid 244 FP) | 0.50% | 0.50% | 0.50% |
| Disintegration Aid | Sodium Chloride (NaCl Powder) | — | 5.00% | — |
| Disintegration Aid | 1:1 Sodium Chloride:Potassium Chloride | — | — | 5.00% |
| Lubricant | Magnesium Stearate | 0.25% | 0.25% | 0.25% |

TABLE 14
Exemplary spray-dried dispersion tablets.

| | | |
|---|---|---|
| | | % w/w |
| Function | Exemplary Ingredients | of Tablet |

Pregranulation

| | | | |
|---|---|---|---|
| Active (SDD) | 15/85 Compound A-HCl/HPMCAS-M, or | 20 to | 35% |
| | 15/85 Compound A-HCl/PVPVA 64, or | | |
| | 35/65 Compound A-HCl/PVPVA 64 | | |
| Filler (s) | Microcrystalline Cellulose, Mannitol | 20 to | 60% |
| Disintegrant(s) | Pregelatinized Starch, Croscarmellose | 5 to | 30% |
| | Sodium, Crospovidone | | |
| Disintegration | Sodium Chloride (NaCl Powder), | 0 to | 10% |
| Aid(s) | 1:1 Sodium Chloride:Potassium Chloride | | |
| Glidant | Silicon dioxide | 0.25 to | 1% |
| Lubricant | Magnesium Stearate | 0.25 to | 1% |

| | | |
|---|---|---|
| Total Pregranulation | 70% to | 85% |

Extragranular

| | | | |
|---|---|---|---|
| Filler (s) | Microcrystalline Cellulose | 0 to | 20% |
| Disintegrant(s) | Croscarmellose Sodium, Crospovidone | 3 to | 10% |
| Disintegration | Sodium Chloride (NaCl Powder), | 0 to | 5% |
| Aid(s) | 1:1 Sodium Chloride:Potassium Chloride | | |
| Glidant | Silicon dioxide | 0.25 to | 1% |
| Lubricant | Magnesium Stearate | 0.25 to | 1% |

| | | |
|---|---|---|
| Total Extragranular | 15 to | 30% |

A representative non-limiting description of the manufacturing process for the SDD tablets is as follows:

Stage 1: Spray Drying: Compound A-HCl and copovidone are dissolved in MeOH. The solution spray-dried. Spray-dried dispersion (Compound A-HCl SDD) is collected.

Stage 2: Roller Compaction: Granulation blend consisting of Compound A-HCl SDD, filler(s), disintegrant(s), glidant(s), and lubricant(s) are blended. In some embodiments, granulation blend consisting of Compound A-HCl SDD, mannitol, microcrystalline cellulose, crospovidone, colloidal silicon dioxide are prepared and blended. The intra-granular portion of the magnesium stearate is screened and added to the granulation blend. The resulting blend is blended. Granulation blend is charged into hopper of the roller compaction and compacted into ribbons. The ribbons are passed through mesh screen using in-line oscillating mill to break up the ribbons and mill into granules.

In some embodiments, the granulation blend comprises about 20% to about 35% (w/w of the final tablet weight) Compound A-HCl SDD. In some embodiments, the granulation blend comprises about 21%, about 22%, about 28%, 29%, about 33%, about 34% (w/w of the final tablet weight) Compound A-HCl SDD. In some embodiments, the Compound A-HCl SDD comprises a 15/85 Compound A-HCl/HPMCAS-M, 15/85 Compound A-HCl/PVPVA64, or 35/65 Compound A-HCl/PVPVA 64 SDD.

Stage 3: Blending: The intra-granular material is mixed with the extragranular excipients. Extragranular excipients comprise one or more excipients selected from: fillers, disintegrants, glidants, and lubricants. The extragranular components include microcrystalline cellulose, crospovidone, colloidal silicon dioxide. The extragranular lubricant, magnesium stearate, is sieved using an appropriately sized screen, then added to the blend and mixed.

Stage 4: Compression: The final blend is compressed into tablets.

Stage 5: Pan-Coating: Film coating suspension of Opadry White 03K18416. is prepared in purified water, and tablets are coated with Opadry White 03K18416 in a perforated coating pan.

### Example 4: Evaluation of Formulation Performance in Dogs

### Study Designs

Evaluated two conditions* in the dog: +Pg pretreatment (mimics human fasted stomach, pH 1-2) and −Pg pretreatment (mimics humans taking PPIs or antacids, pH 3-5). (*: 1-week washout between each condition; Pg=pentagastrin.)

### Compound A-HCl Solution

N=4 non-naïve dog. Vehicle: propylene glycol. Conditions: −Pg.

Compound A-HCl HMG capsule

N=4 non-naïve dog. Conditions: +Pg, −Pg.

### Compound A-HCl Spray-Dried Dispersion Tablets: PVPVA

2 groups of N=6 non-naïve male dogs. Conditions: +Pg, −Pg.

Results from this study is presented in Tables 15 and 16.

TABLE 15
Dog PK evaluation of formulations of Compound A-HCl

| | | 20 mg HMG capsule | | 20 mg PVPVA SDD tablets | |
|---|---|---|---|---|---|
| | Solution | Fasted | Fasted | Fasted | Fasted |
| PK Parameters | (−Pg) | +Pg | −Pg | +Pg | −Pg |
| Cₘₐₓ (ng/mL) | 191 ± 55.2 | 126 ± 99.6 | 14.2 ± 2.9 | 67.5 ± 41.3 | 125 ± 72.6 |
| AUC₀₋ₜ (ng*hr/mL) | 1390 ± 559 | 874 ± 623 | 85.2 ± 32.6 | 366 ± 243 | 454 ± 226 |

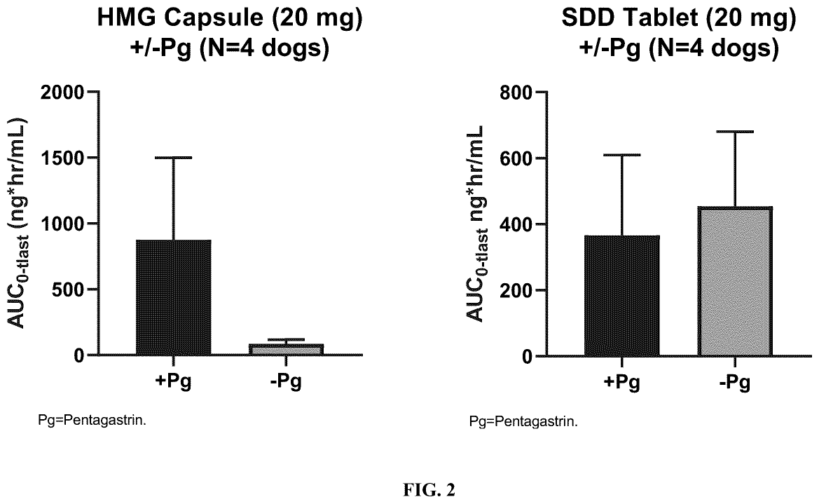

As shown in Table 15 and FIG. 2, the HMG capsule formulations performed poorly in dogs not pretreated with pentagastrin, whereas the spray-dried dispersions tablets performed better; For HMG capsule formulation, AUC without pentagastrin was only 11% of the AUC with pentagastrin (98.2 ng*hr/mL compared to 917 ng*hr/mL). In comparison, the AUC for the PVPVA SDD tablet formulations without pentagastrin was 185% and 124% of with pentagastrin condition, respectively. These data show that PVPVA SDD tablet formulations are superior under high gastric pH environment (e.g., as would be in subjects that are are taking PPI or antacids).

TABLE 16
Dog PK evaluation of 60 mg 35/65 PVP-VA SDD tablet of
Compound A-HCl

| | 35% SDD | 60 mg PVPVA SDD tablet | | |
|---|---|---|---|---|
| PK Parameters | Suspension | Tablet C3 | Tablet C4 | Tablet C5 |
| Cₘₐₓ (ng/mL) | 437 ± 140 | 377 ± 129 | 329 ± 144 | 357 ± 146 |
| AUC₀₋ₜ | 3630 ± 1990 | 3110 ± 1360 | 2540 ± 1120 | 2560 ± 1140 |
| (ng*hr/mL) | | | | |

### Example 5: A Double-Blind, Randomized, Placebo-Controlled, Single-and-Multiple-Dose Study to Evaluate the Safety, Pharmacokinetics, and Pharmacodynamics of Compound a in Healthy Volunteers

A phase 1, randomized, double-blind, placebo-controlled, single and multiple ascending dose study was performed with oral Compound A at a single study center. Inclusion criteria included age 18 to 50 years; body mass index of 18-30 kg/m²; and if female, postmenopausal by history or surgery. Exclusion criteria included presence of significant medical history.

Screening occurred within 28 days prior to the first administered dose (Day 1) in both single-dose and multiple-dose cohorts. Dosing of paltusotine or placebo commenced on Day 1 after baseline studies. Participants fasted overnight for ≥10 h prior to each dose administration and remained fasting until 4 h post-dose (after collection of the last GH sample after growth hormone-releasing hormone [GHRH] challenge). For the food effect cohort, the study drug was administered 30 min after a high-fat and high-calorie breakfast.

Study drug for administration was provided as either an oral solution (prepared on site at the Phase 1 unit) or as capsules (placebo, 5 mg, and 20 mg) of Compound A-HCl.

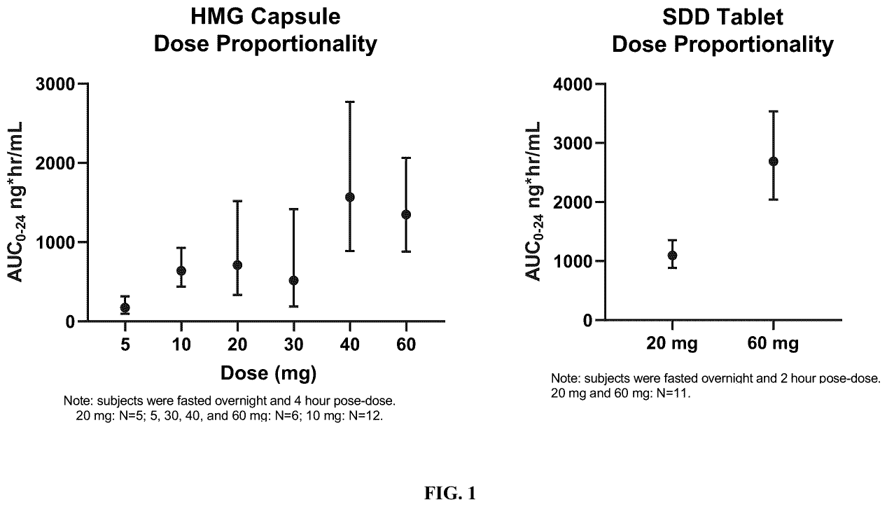

Participants received oral study drug or placebo according to the scheme shown in FIG. 1. In the single-dose phase, participants were randomized 6:2 to Compound A (1.25, 2.5, 5, 10, 20, 40, and 60 mg) or placebo in 7 cohorts (S1 to S7). Cohorts comparing oral bioavailability of solution versus capsules and the effect of a standardized high-fat, high-calorie breakfast on oral bioavailability of the capsule formulation employed the 10 mg dose level. In the multiple-dose phase, participants were randomized 6:3 to once-daily oral Compound A-HCl capsules or placebo at doses of 5 mg for 7 days (M1, Day Last=Day 7), and 10, 20, and 30 mg daily for 10 days (M2, M3, M4, Day Last=Day 10).

Pharmacokinetic sampling: Single-dose cohorts: Plasma samples were collected on Day 1 (pre-dose, every 15 min up to 90 min post-dose, then at 2, 3, 4, 6, 8, 10, 12, 18, 24, 48, 72, 96, 120, and 144 h post-dose). Multiple-dose cohorts: For the 5 mg cohort, plasma samples were collected on Day 1 (pre-dose, every 15 min up to 90 min post-dose, then at 2, 3, 4, 6, 8, 10, 12, and 18 h post-dose), Days 2-6 (pre-dose and 2 h post-dose), Day 7 (same time points as Day 1, followed by samples at 18, 24, 48, 72, 96, 120, and 144 h post-dose). For all other multiple-dose cohorts, plasma samples were collected on Day 1 (pre-dose, every 15 min up to 90 min post-dose, then at 2, 3, 4, 6, 8, 10, 12, and 18 h post-dose), Days 2-6 (pre-dose and 2 h post-dose), Day 10 (same time points as Day 1, followed by samples at 18, 24, 48, 72, 96, 120, 144, 192, and 240 h post-dose). The last time point at which blood sample was collected for pharmacokinetics and terminal elimination half-life (t_{1/2}) determination was 144 h post-dose for single-dose cohorts and cohort M1 and 240 h post-dose for cohorts M2-M4.

Assessment of GH: To assess the ability of paltusotine to suppress GHRH-stimulated GH secretion, a GHRH challenge was performed in single-dose cohorts S1, S2, S3, S4, and S5 (oral solution). Recombinant human GHRH(1-44)-NH2 (somatorelin; Ferring Pharmaceuticals, Parsippany, NJ) 50 μg was administered intravenously 2 h after paltusotine (or placebo) on Day 1 and at approximately the same time on Day −1 (±10 min). A blood sample for serum GH was collected at −60 min, −30 min, immediately before GHRH challenge, and at 15, 30, 45, 60, 90, and 120 minutes post-challenge. Serum GH was also assessed in the M1 cohort. Serum GH sample was collected on Day −1, on Days 1 and 7 (pre-dose, 0.5, 1, 2, 3, 4, 6, 8, 10, 12 and 18 h post-dose), Days 2-6 (pre-dose and 12 h post-dose), and Days 8, 10 and 14 (at approximately the same time as dosing).

Assessment of IGF-1: For assessment of serum IGF-1, in the single-dose cohorts, serum IGF-1 was collected on Day −1 and Day 1 (pre-dose to 18 h post-dose), and Day 2 and 7 (at approximately the same time GHRH was administered on Day 1). In the M1 cohort, IGF-1 samples were obtained pre-dose, 6 and 12 h post-dose on Days −1, 1 and 7; pre-dose and 12 h post-dose on Days 2-6; and at approximately the same time as dosing on Days 8, 10, and 14. For M2 and subsequent cohorts, IGF-1 samples were obtained pre-dose, 6 and 12 h post-dose on Days −1, 1 and 10; pre-dose and 12 h post-dose on Days 2-9; and at approximately the same time as dosing on Days 11, 14, and 21.

Safety assessments: Safety assessments included monitoring of clinical laboratory tests, cardiovascular safety monitoring (continuous Holter monitoring, ECG, telemetry), and physical examinations conducted at scheduled times throughout the study in the research units as well as post-discharge (up to 9 days after the single-dose, or up to 11 days after the last dose for multiple-dose cohorts). Adverse events and concomitant medications were recorded. Laboratory samples for clinical chemistry, hematology, coagulation tests, and urinalysis were collected under fasted conditions.

In the multiple-dose cohorts, baseline serum TSH, ACTH, and prolactin samples were collected on Day −2, and an oral glucose tolerance test (OGTT) was conducted on Day −7 or earlier. These assessments were repeated on Day 8 (M1) or Day 11 (M2 and subsequent cohorts).

Oral glucose tolerance test (75 g glucose load) was administered to healthy subjects at baseline and after repeat dosing with Compound A. No subject demonstrated abnormal blood glucose levels prior to glucose challenge at baseline (Day −7). Clinically significant hypoglycemia occurred in 1 subject in Cohort M3 (Compound A-HCl−10 days at 20 mg/day) at 2 and at 3 hours post-glucose challenge, and in 1 subject in Cohort M4 (Compound A-HCl−10 days at 30 mg/day) at 3 hours post-glucose challenge at the baseline assessment (Day −7).

Two TEAEs of asymptomatic hyperglycemia occurred. Both events occurred 1 hour post oral glucose and resolved by 2 hours post load during the scheduled OGTT following the completion of 10 days of Compound A dosing. One subject in Cohort M3 experienced a peak glucose concentration of 12.2 mmol/L (220 mg/dL) 1 hour post oral glucose challenge which normalized to 5.8 mmol/L (104 mg/dL) by 2 hours post challenge. One subject in Cohort M2 (Compound A-HCl−10 days at 10 mg/day) experienced a peak glucose concentration of 12.4 mmol/L (223 mg/dL) 1 hour post oral glucose challenge which normalized to 4.3 mmol/L (77 mg/dL) by 2 hours post-challenge. No symptoms of hyperglycemia or other sequelae were reported in these cases. The clinical significance of isolated 1 hour post oral glucose challenge hyperglycemia is unclear.

Treatment with up to 30 mg Compound A was associated with increases (up to 36% increase from baseline) serum glucose AUC_{0-3 hr}, compared to 18% increase from baseline for placebo. This effect was not dose dependent and was not associated with significant changes in fasting plasma glucose.

Differences in glucose AUC_{0-3 hr} from baseline to the day after the last administered dose of study drug was significant for cohorts M3 (Compound A-HCl −10 days at 20 mg/day; 95% CI 1.8, 8.4; p=0.0104) and M4 (Compound A-HCl −10 days at 30 mg/day; 95% CI 1.8, 4.6; p=0.0021), however, this finding is limited by the low number of subjects and observations (see the following Table).

Comparison of Oral Glucose Tolerance Test AUC (Multiple-Dosing) -
Safety Population

| Parameter | | | Difference | 95% CI for | |
|---|---|---|---|---|---|
| (unit) | Treatment Group | N | (SE) | Difference | p value |
| Glucose AUC | Compound A-HCl | 5 | 3.8 (4.00) | (−1.2, 8.8) | 0.1013 |
| (0-3 hr) | 7 days - 5 mg/day | | | | |
| (h*mmol/L) | Compound A-HCl | 6 | 2.4 (3.19) | (−1.0, 5.7) | 0.1294 |
| | 10 days - 10 mg/day | | | | |
| | Compound A-HCl | 6 | 5.1 (3.12) | (1.8, 8.4) | 0.0104 |
| | 10 days - 20 mg/day | | | | |
| | Compound A-HCl | 6 | 3.2 (1.34) | (1.8, 4.6) | 0.0021 |
| | 10 days - 30 mg/day | | | | |
| | All Placebo | 12 | 1.5 (4.85) | (−1.5, 4.6) | 0.2956 |

Note:
Paired t-test is used for within-group comparison, while Difference = (AUC at Day −7) minus (AUC at day after last Compound A dose).
SE = standard error of the mean.

Analytical methods: Serum was isolated, stored, and analyzed for IGF-1 and GH according to the instructions from the manufacturer (IGF-1 and human GH diagnostic assay, Siemens Immulite® 1000 systems).

Plasma pharmacokinetic sample analysis for Compound A was determined using validated procedures by CPR Pharma Services (Thebarton, Australia). Briefly, plasma Compound A concentrations were determined by high-pressure liquid chromatography (HPLC) coupled with tandem mass spectrometry detection (MS/MS). Deuterated Compound A was used as an internal standard. Supported liquid extraction was used to extract Compound A and the internal standard. The analyte was separated by HPLC using an ACE C18-AR column (Advanced Chromatography Technologies, Ltd), and the eluates were monitored by an API 4000™ LC-MS/MS System (Sciex, Framingham, MA) in positive multiple reaction monitoring mode. The extract was then assayed against a calibration curve. The data were acquired and integrated by the data acquisition software AnalystR (Sciex), linked directly to the API 4000™ LC-MS/MS System, and then processed using Watson LIMS™ software (Thermo Scientific), where applicable. The calibration range was 0.100 to 100 ng/mL using 100 μL of plasma, with a lower limit of quantification of 0.100 ng/mL. The inter-assay accuracy was 94% to 100%, and the inter-assay precision (% CV) was between 6.1% and 15%.

Statistical analysis: The study endpoints were effects of Compound A on GHRH-stimulated GH and IGF-1 (pharmacodynamics), pharmacokinetics (including formulation and food effects), safety, and tolerability. For the analysis of effects Compound A on GHRH-stimulated GH and IGF-1, the pharmacodynamic population included all randomized participants in the 1.25 mg, 2.5 mg, 5 mg, 10 mg, and 20 mg (S1, S2, S3, S4A, and S5) single-dose cohorts who received any amount of study drug and had at least one post-baseline pharmacodynamic assessment. The percent change in serum GH area under the curve (AUC) from 0 to 2 h (AUC₀₋₂) from baseline was compared using ANOVA between each dosing cohort and all participants who received placebo. The single dose pharmacodynamic population included 39 participants from the S1, S2, S3, S4, and S5 cohorts (paltusotine n=29; placebo n=10). One participant each in the M1, M3, and M4 cohorts, and two placebo-treated participants did not meet the criteria for inclusion in the pharmacodynamic population because they did not receive all doses. For multiple-dose cohorts, only participants who received all doses were included. A change in IGF-1 levels 12 h post last dose from baseline (average of 3 or 4 measurements) were compared by ANOVA between each dosing cohort and all participants who received placebo. The multiple dose pharmacodynamic population included 31 participants from the M1, M2, M3, and M4 cohorts (Compound A n=21; placebo n=10).

For assessing pharmacokinetics of Compound A, the single-dose pharmacokinetic population included all randomized participants who received any amount of study drug with sufficient plasma concentration data. For the multiple-dose cohorts, only participants who received all doses were included. The relative bioavailability of the capsule formulation was determined by comparing its pharmacokinetic parameters with those of the solution formulation at the 10 mg dose. Food effect was determined at the same dose by comparing pharmacokinetic parameters in the fasting state to those when Compound A was administered with a high-fat, high-calorie meal. For both formulation and food effect, ANOVA with treatment as a fixed effect and subject as a random effect was performed. Data for peak concentration (Cₘₐₓ), AUC₀₋ₗₐₛₜ, and AUC_{0-inf} were natural log-transformed.

### Example 6: A Phase 1, Multi-Cohort, Single Dose Study to Assess the Relative Bioavailability, Performance, and Safety of Two Formulations of Compound A

The study was conducted in up to 3 cohorts, each with a specific primary objective:
Cohort 1: To characterize performance of 10 mg tablets prepared by spray-dried dispersion (SDD) of Compound A-HCl salt.
Cohort 2: To evaluate the relative bioavailability of 10 mg SDD tablets compared to the Compound A-HCl hot melt granulation (HMG) formulation, 10 mg capsules. To determine the effect of timing of food administration on pharmacokinetics of low dose of the 10 mg SDD tablets.
Cohort 3: To determine the effect of timing of food administration on pharmacokinetics of SDD tablets and dose proportionality in doses higher than 20 mg. To determine the optimal dosing regimen that results in adequate systemic exposure with short post-dose fasting duration.
Cohort 4: To determine the effect of timing of food administration on pharmacokinetics of SDD tablets in doses higher than 20 mg. To determine the optimal dosing regimen of paltusotine that results in high systemic exposure with low post-dose fasting duration
Cohort 5: To determine the effect of the proton-pump inhibitor (PPI), lansoprazole, on the pharmacokinetics of a 60 mg dose of SDD tablets. To determine the effect of a low-fat meal on the pharmacokinetics of a 60 mg dose of SDD tablets.

### Study Design:

Up to thirty-six (36) healthy male and female subjects were enrolled. Cohorts 1-2 consisted of four periods each, and Cohort 3 consisted of three periods.

### Cohort 1:

The SDD tablets were evaluated. Up to twelve (12) healthy male and female subjects were enrolled in each cohort. Cohort 1 consisted of 4 periods: In Period 1, subjects were administered a proton-pump inhibitor (lansoprazole, 15 mg BID for 3 days (from Day −3), taken orally at least 30 min prior to a meal, once in the morning and once in the evening). On the fourth day (Day 1 of study), fasted subjects will take the last dose of lansoprazole (15 mg) 60 min prior to 20 mg Compound A (2×10 mg of the SDD tablets). In Period 2, fasted subjects were administered 20 mg Compound A (2×10 mg of the SDD tablets). In Period 3, fasted subjects were administered 20 mg Compound A (2×10 mg of the SDD tablets) with a high-fat, high-calorie meal. In Period 4, fasted subjects will take up to 80 mg Compound A (up to 8×10 mg of the SDD tablets). The actual dose was selected based on the pharmacokinetic data from Period 2.

For Period 1: In the evening before dosing (Day −1), subjects were administered their evening dose of 15 mg lansoprazole, provided an evening meal at least 30 min after administration of lansoprazole, and then were required to fast overnight (≥10 hr) on Day −1. On Day 1, they were administered the morning dose (last dose) of 15 mg lansoprazole at least 60 min prior to administration of Compound A (2×10 mg SDD tablets). Subjects continued to fast for 2 hr after Compound A, after which they were allowed to ingest a standard meal.

For Period 2: Subjects were required to fast overnight (≥10 hr) on Day 7. On Day 8, 20 mg Compound A (2×10 mg SDD tablets) was administered orally. Subjects continued to fast for 2 hr after Compound A, after which they were allowed to ingest a standard meal.

For Period 3: Subjects were required to fast overnight (≥10 hr) on Day 14. On Day 15, they were allowed to ingest a high-fat, high-calorie meal within 30 min. Upon completion of the ingestion of the meal, Compound A (2×10 mg SDD tablets) was administered (no more than 30 min after the start of the meal). No additional food was provided for at least 4 hr after administration of Compound A.

Subjects were not allowed to perform strenuous exercise of >30 min/day 3 days prior to Day −1 and throughout the study.

PK and safety assessments including adverse event (AE) monitoring, clinical laboratory tests, vital sign measurements, 12-lead ECGs, Holter and telemetry monitoring (Period 4 only), and physical examinations were conducted at scheduled times throughout the study.

### Cohort 2:

The cohort consisted of four periods. In each period, a single dose of 20 mg Compound A (2×10 mg SDD) was administered orally.

For Period 1: Subjects were required to fast overnight (≥10 hr) on Day −1. On Day 1, a low-fat meal was given 2 hours after administration of 20 mg Compound A (2×10 mg HMG capsules; reference formulation).

For Period 2: Subjects were required to fast overnight (≥10 hr) on Day 7. On Day 8, they were given a low-fat meal 2 hr after administration of 20 mg Compound A (2×10 mg SDD tablets; test formulation).

For Period 3: Subjects were required to fast overnight (≥10 hr) on Day 14. On Day 15, they were given a low-fat meal 1 hr after administration of 20 mg Compound A (2×10 mg SDD tablets).

For Period 4: Subjects were required to fast overnight (≥10 hr) on Day 21. On Day 22, they were given a low-fat meal 0.5 hr after administration of 20 mg Compound A (2×10 mg SDD tablets).

The final study visit occurred on Day 29. Subjects were not allowed to perform strenuous exercise of >30 min/day, 3 days prior to Day −1 and throughout the study. PK and safety assessments including adverse event (AE) monitoring, clinical laboratory tests, vital sign measurements, 12-lead ECGs, and physical examinations were conducted at scheduled times throughout the study.

### Cohort 3:

The cohort consisted of three periods. In each period, a single dose of Compound A SDD (40, 60, or 80 mg) was administered orally (4×10 mg SDD tablets, 6×10 mg SDD tablets, or 8×10 mg SDD tablets). There was a washout period of at least 10 days between each dose of Compound A.

For Period 1: Subjects were required to fast overnight (≥10 hr) on Day −1. On Day 1, they were given a standard meal 1 hr after administration of 40 mg Compound A (4×10 mg SDD tablets).

For Period 2: Subjects were required to fast overnight (≥10 hr) on Day 10. On Day 11, they were given a standard meal 1 hr or 2 hr after administration of 80 Compound A (8×10 mg SDD tablets). The timing of the meal (1 hr or 2 hr post administration of Compound A) was dependent on the mean AUC₀₋₂₄ determined in Period 1.

For Period 3: Subjects were required to fast overnight (≥10 hr) on Day 20. On Day 21, they were given a standard meal 1 hr or 4 hr after administration of 60 or 80 mg Compound A (6×10 mg SDD tablets or 8×10 mg SDD tablets). The dose and timing of the standard meal was dependent on the mean AUC₀₋₂₄ determined in Period 2.

The final study visit occurred on Day 29. Subjects were not allowed to perform strenuous exercise of >30 min/day, 3 days prior to Day −1 and throughout the study. PK and safety assessments including adverse event (AE) monitoring, clinical laboratory tests, vital sign measurements, 12-lead ECGs, and physical examinations were conducted at scheduled times throughout the study.

### Cohort 4:

Cohort 4 consisted of 3 periods as follows:

In Period 1, fasted subjects were given a standard meal 1 hour after administration of 40 mg (4×10 mg) paltusotine SDD tablets.

In Period 2, fasted subjects were given a standard meal 1 hour after administration of 80 mg (8×10 mg) paltusotine SDD tablets.

In Period 3, fasted subjects were given a standard meal 4 hours after administration of 80 mg (8×10 mg) paltusotine SDD tablets.

### Cohort 5:

The cohort consisted of three periods. The PK of 60 mg paltusotine administered as 3×20 mg SDD tablets was assessed using the following criteria: effect of a low-fat meal on the PK of paltusotine (Period 1 and Period 3) and effect of the PPI lansoprazole on the PK of paltusotine (Period 2) as follows:

In Period 1, fasted subjects were given a low-fat meal 1 hour after administration of 60 mg (3×20 mg) paltusotine SDD tablets. Low-fat meal: 400-500 calories such that 25% of the calories are derived from fat (11-14 grams).

In Period 2, subjects were administered lansoprazole (15 mg BID for 3 days, taken orally at least 30 min prior to a meal). On the fourth day, fasted subjects were administered the last dose of lansoprazole (15 mg) 60 min prior to administration of 60 mg paltusotine (3×20 mg SDD tablets). Subjects continued to fast for 1 hour after paltusotine administration, after which they were provided a low-fat meal. Subjects remained fasted until 4 hours following dose administration.

In Period 3, fasted subjects ingested a low-fat meal within 30 minutes. Upon completion of ingestion of the meal (but no more than 30 min after the start of the meal), subjects were dosed with 60 mg paltusotine (3×20 mg SDD tablets). Subjects remained fasted until 4 hours following dose administration

### Study Population:

Up to 36 healthy male or female subjects, between the ages of 18 to 55 years, inclusive, were enrolled. For Cohort 2 only, male and female subjects 18 to 65 years of age, inclusive, at the time of screening.

### Inclusion Criteria

Each subject had to meet all of the following inclusion criteria to be enrolled in the study: Male and female subjects 18 to 55 years of age, inclusive, at the time of screening. For cohort 2 only, male and female subjects 18 to 65 years of age, inclusive, at the time of screening. Body mass index (BMI) of 18 to 30 kg/m², inclusive. Willing to refrain from strenuous, unaccustomed exercise and sports, defined as greater than 30 minutes per day, 3 days prior to Day −1 and throughout the study. If the subject was a heterosexual or bisexual female, she had to be of non-childbearing potential OR must agree to use a highly effective or two clinically acceptable methods of contraception.

### Exclusion Criteria

A healthy subject meeting any of the following criteria was to be excluded from the study: Prior treatment with Compound A. Any uncontrolled or active major systemic disease which makes study participation unsafe or could interfere with evaluation of the endpoints of the study. History or presence of malignancy except adequately treated basal cell or squamous cell carcinomas of the skin within the past 5 years. Active acute or chronic infection. Use of any investigational drug within the past 60 days or 5 half-lives, whichever is longer, prior to the first dosing of study drug. Use of tobacco and/or nicotine-containing products, recreational drugs, or alcohol for 48 hr prior to admission and agreement to refrain from use throughout the study. History of or current alcohol abuse and/or other drug addiction <1 year prior to screening. Used any prescription or over-the-counter (OTC) medication or alternative medicinal products within 14 days of Day −1. Use of caffeine-containing beverages or food for 48 hr prior to Day −1 and for 48 hr prior to each check-in day for all subsequent periods. Have ingested foods containing poppy seeds within 7 days before screening until completion of the study assessments. Taking moderate or strong CYP3A4 inhibitors or inducers. Strenuous exercise for >30 min/day, 3 days prior to Day −1 and throughout the study. Had a blood loss ≥500 mL or donated blood within 3 months prior to admission. Have amylase and/or lipase levels >2×ULN, alanine aminotransferase (ALT) and/or aspartate aminotransferase (AST) >2×ULN, total bilirubin >1.5×ULN (except in the case of known Gilbert's syndrome), and/or serum creatinine above the upper limit of normal. History of hypersensitivity reactions to any excipients in the study drug. Tested positive at screening for human immunodeficiency virus (HIV), hepatitis B surface antigen (HBsAg), or hepatitis C antibody (HCV-Ab), or has a history of a positive result. Female subjects who have a positive serum pregnancy test or are breastfeeding. For Cohort 1 only, subjects that are classified as CYP2C19 poor metabolizers or ultra-rapid metabolizers.

### Test Product, Dose, and Mode of Administration:

10 mg tablets (SDD). Multiple tablets were swallowed with water depending on dose specified for a given period/cohort.

20 mg tablets (SDD). Multiple tablets were swallowed with water depending on dose specified for a given period/cohort.

### Reference Therapy, Dose, and Mode of Administration:

10 mg HMG capsule formulation served as the reference formulation. Multiple capsules were swallowed with water depending on dose specified for a given period/cohort.

### Plasma Pharmacokinetic Parameters:

Blood PK samples were collected to evaluate Compound A plasma concentrations.

PK parameters were calculated for Compound A and the following are shown in tables below: Area under the plasma concentration curve from 0 to 24 hours (AUC₀₋₂₄); Maximum plasma concentration (Cₘₐₓ); Time to achieve maximum plasma concentration (Tₘₐₓ).

### Results

Results from this clinical trial showed that the co-administration of proton pump inhibitors only had a small effect on the pharmacokinetics observed with the administration of the SDD tablets, shorter fasting times are realized with the SDD tablets and the SDD tablets provide better dose proportional pharmacokinetics.

Results from Cohort 1 is presented in Table 17.

TABLE 17
Results from Cohort 1

| | | |
|---|---|---|
| Mean | SD | CV % |

Period 1 (SDD 20 mg + PPI)

| Tₘₐₓ (hr) | 2 (1.5-3) | | |
|---|---|---|---|
| Cₘₐₓ (ng/mL) | 92.5 | 21.6 | 23.3 |
| AUC₀₋₂₄ (hr*ng/mL) | 891 | 240 | 26.9 |

Period 2 (SDD 20 mg alone)

| Tₘₐₓ (hr) | 3 (1.5-6) | | |
|---|---|---|---|
| Cₘₐₓ (ng/mL) | 114 | 40.6 | 35.5 |
| AUC₀₋₂₄ (hr*ng/mL) | 1140 | 337 | 29.5 |

Period 3 (SDD 20 mg + food)

| Tₘₐₓ (hr) | 1.8 (1.3-4) | | |
|---|---|---|---|
| Cₘₐₓ (ng/mL) | 16.7 | 6.82 | 40.9 |
| AUC₀₋₂₄ (hr*ng/mL) | 155 | 46.3 | 29.9 |

Period 4 (SDD 60 mg)

| Tₘₐₓ (hr) | 3 (1-6) | | |
|---|---|---|---|
| Cₘₐₓ (ng/mL) | 305 | 139 | 45.4 |
| AUC₀₋₂₄ (hr*ng/mL) | 2900 | 1240 | 42.6 |

P4/P2 Ratio

| | |
|---|---|
| Cₘₐₓ | 3.0 (1.7-3.4) |
| AUC₀₋₂₄ | 2.8 (1.6-3.7) |

Median and range are reported for Tₘₐₓ. Mean and range are reported for P4/P2 ratios. All dose administered with overnight fast and 2 h post dose fast. Food: high fat meal.

Cohort 1 (SDD 10 mg×2 under different conditions): the observed exposures with and without PPI are fairly comparable. Cohort 1 (SDD 10 mg×2 vs. 10 mg×6): Relatively dose proportional increases in exposures were observed.

SDD tablets exhibited dose proportional increase in total systemic exposure (AUC) up to a dose of 80 mg.

In comparison to the SDD tablets, relatively dose proportional increases in exposures were not observed with the HMG capsules. See FIG. 1. Dose proportionality data for the HMG capsules obtained from a previous clinical study is presented in Table 18.

TABLE 18
Comparative data: Dose Proportionality Observed for HMG Capsule
Formulations after single dose (4 h post dose fast).

| Parameter | | 5 mg | 10 mg | 20 mg | 30 mg | 40 mg | 60 mg |
|---|---|---|---|---|---|---|---|
| (Mean) | Day | (n = 5) | (n = 6) | (n = 5) | (n = 5) | (n = 6) | (n = 6) |
| Tmax (hr) | 1 | 1.2 ± 0.11 | 1.8 ± 0.94 | 2.4 ± 0.82 | 1.4 ± 0.91 | 3.4 ± 1.1 | 3.0 ± 1.2 |
| Cₘₐₓ | 1 | 16.8 ± 7.22 | 78.7 ± 45.3 | 88.7 ± 43.3 | 78.2 ± 60.7 | 185 ± 118 | 154 ± 77.6 |
| (ng/mL) | | | | | | | |
| AUC₀₋₂₄ | 1 | 167 ± 73.4 | 661 ± 340 | 811 ± 409 | 578 ± 411 | 1770 ± 888 | 1450 ± 656 |
| (ng hr/mL) | | | | | | | |

Data shown are mean ± standard deviation

Results from Cohort 2 is presented in Table 19.

TABLE 19
Results from Cohort 2

| | | |
|---|---|---|
| Mean | SD | CV % |

Period 1 (HMG 2 h fast)

| Tₘₐₓ (hr) | 2 (0.75-3) | | |
|---|---|---|---|
| Cₘₐₓ (ng/mL) | 123 | 54.4 | 44.4 |
| AUC₀₋₂₄ (hr*ng/mL) | 1060 | 462 | 43.6 |

Period 2 (SDD 2 h fast)

| Tₘₐₓ (hr) | 2 (1-3) | | |
|---|---|---|---|
| Cₘₐₓ (ng/mL) | 97.2 | 32.8 | 33.7 |
| AUC₀₋₂₄ (hr*ng/mL) | 877 | 320 | 36.5 |

Period 3 (SDD 1 h fast)

| Tₘₐₓ (hr) | 1.4 (0.75-4) | | |
|---|---|---|---|
| Cₘₐₓ (ng/mL) | 89.3 | 37.0 | 41.4 |
| AUC₀₋₂₄ (hr*ng/mL) | 721 | 319 | 44.2 |

Period 4 (SDD 30 min fast)

| Tₘₐₓ (hr) | 1.3 (0.75-4) | | |
|---|---|---|---|
| Cₘₐₓ (ng/mL) | 81.3 | 26.8 | 32.9 |
| AUC₀₋₂₄ (hr*ng/mL) | 630 | 212 | 33.6 |

P3/P2 Ratio (%)

| | |
|---|---|
| Cₘₐₓ | 92% |
| AUC₀₋₂₄ | 82% |

All dose administered with overnight fast.

Cohort 2 (SDD 10 mg×2 vs. HMG and under different post-dose fasting durations): SDD tablets did not appear to have belier exposures than HMG capsules and the two formulations were relatively comparable. For the SDD tablets the AUC₀₋₂₄ (a measure of extent of absorption) after a 1 hr post dose fast was decreased to 82% of that observed with 2 hr post-dose fast, which is a relatively small decrease in exposure.

In comparison to the performance of the SDD tablets under different post-dose fasting duration scenarios, the HMG capsules performed poorly under different post-dose fasting duration scenarios in a previously completed clinical study. Pharmacokinetic data obtained after administering a 20 mg dose (10 mg HMG capsule ×2) in 12 subjects (N=4 male, N=8 Female) is presented in Table 20.

TABLE 20
Comparative Data: HMG capsule Performance Under Different Post-Dose
Fasting Durations

| Post-Dose Fasting | | | AUC₀₋₂₄ |
|---|---|---|---|
| Duration | Tₘₐₓ (Hr) | Cₘₐₓ (ng/mL) | (Hr* ng/mL) |
| 4 hour | 0.75-6 | 134 (48.9) | 1280 (344) |
| 2 hour | 0.75-3 | 104 (35.5) | 930 (293) |
| 1 hour | 0.75-3 | 92.2 (30.6) | 654 (244) |
| Ratio (%): 2 hr/1 hr | — | 89% | 70% |

Cmax and AUC₀₋₂₄ data show are mean (standard deviation). Range is shown for Tmax.

With the HMG capsule formulations, approximately 30% loss in extent of absorption was noted with a 1-hour post-dose fasting vs. a 2-hour post-dose fast.

HMG capsules with a 2 h fast was evaluated in Phase 2 clinical studies. A 1 hour fast is more desirable than a 2 hour fast. Only 18% loss of AUC₍₀₋₂₄₎ was observed with 1 hour fast compared to a 2 hour fast. SDD 1 h fast will be utilized for Phase 3. Importantly, SDD tablets appeared to have better dose proportionality than HMG capsules, allowing for 3.0× dose (i.e., 60 mg) to be administered in Phase 3 clinical studies.

Results from Cohort 5 is Presented in Table 21.

TABLE 21
Results from Cohort 5 (PPI)

| | | | Cₘₐₓ | AUC_{0-inf} | Tₘₐₓ |
|---|---|---|---|---|---|
| Period | Treatment | N | (ng/mL) | (hr*ng/mL) | (hr) |
| 1 | 60 mg | 12 | 250 | 4050 | 2.0 |
| | | | (47.5%) | (46.4%) | [1.0-4.0] |
| 2 | 60 mg + PPI | 12 | 153 | 2450 | 2.0 |
| | | | (48.8%) | (58.2%) | [1.3-6.0] |

Mean (CV %), except Tₘₐₓ where median [range] are reported.

The exposures (Cₘₐₓ and AUC) of Compound A appeared to be reduced by ˜40% when administered with PPI.

Additional results from Cohort 5 is presented in Table 22.

TABLE 22
Results from Cohort 5 (Low-fat meal)

| | | | Cₘₐₓ | AUC_{0-inf} | Tₘₐₓ |
|---|---|---|---|---|---|
| Period | Treatment | N | (ng/mL) | (hr*ng/mL) | (hr) |
| 1 | 60 mg | 11 | 256 | 4140 | 2.0 |
| | | | (47.8%) | (46.8%) | [1.0-4.0] |
| 3 | 60 mg + Low-fat | 11 | 104 | 1650 | 2.0 |
| | meal | | (63.9%) | (55.1%) | [1.3-4.0] |

Mean (CV %), except Tₘₐₓ where median [range] are reported.

The exposures (Cₘₐₓ and AUC) of Compound A appeared to be reduced by ˜60% when administered with low-fat meal, compared to >80% reduction when administered with high-fat meal.

### Example 7: A Phase 2, Open Label Exploratory Study to Evaluate the Safety, Pharmacokinetics and Efficacy of Compound a in Patients with Acromegaly Treated with Somatostatin Analogue Based Treatment Regimens (ACROBAT Edge)

An open label exploratory study designed to evaluate the safety, efficacy, and pharmacokinetics of Compound A (also known as paltusotine) in subjects with acromegaly that are treated with somatostatin analogue (SSA) based treatment regimens.

### Study Design:

Intervention Model: Single Group Assignment. Masking: None (Open Label). Primary Purpose: Treatment.

### Test Product, Dose, and Mode of Administration:

5 mg or 10 mg HMG capsule formulation. Multiple capsules were swallowed with water depending on dose specified for a given period/cohort. Enrolled patients began treatment with oral paltusotine 10 mg/day. The dose was up-titrated in a double-blind manner in 10-mg increments to a maximum 40 mg/day at weeks 4, 7 and 10 according to IGF-1 levels measured at weeks 2, 5, and 8, respectively. Up-titrations were performed if the investigator confirmed that the patient was tolerating the current dose and the unblinded central IGF-1 reader determined that IGF-1 was >0.9×ULN at week 2 and 5 or >1.0×ULN at week 8. Dose decreases to the prior dose level (eg, 20 mg to 10 mg, 10 mg to 5 mg) were to occur if patients did not tolerate the current dose. No dose changes were made if IGF-1 suppression into the normal range was achieved with a tolerated dose

### Outcome Measures

Primary Outcome Measures: Change from baseline (mean of Screening values) in insulin-like growth factor-1 (IGF-1) level [Time Frame: 13 Weeks].

Secondary Outcome Measures: 1) Proportion of subjects with their last IGF-1 measurement ≤upper limit of normal (ULN) [Time Frame: 13 Weeks]. 2) Proportion of subjects with their last IGF-1 measurements ≤1.5×ULN [Time Frame: 13 Weeks].

### Eligibility Criteria

### Inclusion Criteria

Male and female subjects 18 to 70 years of age. Confirmed diagnosis of acromegaly with either a partial or complete response to protocol defined somatostatin analogue therapy regimens. Females must be non-pregnant and non-lactating, and either surgically sterile, post-menopausal, or using effective method(s) of birth control. Willing to provide informed consent.

### Exclusion Criteria

Treatment naïve acromegaly subjects. Prior treatment with paltusotine. Pituitary surgery within 6 months prior to Screening. Subjects receiving radiation therapy may be eligible with some restrictions. History or presence of malignancy except adequately treated basal cell and squamous cell carcinomas of the skin within the past 5 years. Use of any investigational drug within the past 30 days or 5 half-lives, whichever is longer. Positive test at Screening for HIV, hepatitis B surface antigen (HBsAg) or hepatitis C antibody (HCV-Ab) or has a history of a positive result. History of alcohol or substance abuse in the past 12 months. Any condition that in the opinion of the investigator would jeopardize the subject's appropriate participation in this study. Cardiovascular conditions or medications associated with prolonged QT or those which predispose subjects to heart rhythm abnormalities. Subjects with symptomatic cholelithiasis. Subjects with clinically significant abnormal findings during the Screening Period, and any other medical condition(s) or laboratory findings that, in the opinion of the Investigator, might jeopardize the subject's safety or ability to complete the study. Subjects taking octreotide LAR at a dose higher than 40 mg, or lanreotide depot at a dose higher than 120 mg, or pasireotide LAR at a dose higher than 60 mg. Subjects who usually take octreotide LAR or lanreotide depot less frequently than every 4 weeks (e.g. every 6 weeks or 8 weeks).

### Patient Reported Outcomes

Patients recorded daily acromegaly symptoms using a nine-item Acromegaly Symptom Diary (ASD). The ASD is a 9-item patient-reported measure of symptoms associated with acromegaly, including headache, joint pain, sweating, fatigue, leg weakness, swelling, numbness/tingling, sleep difficulties, and short-term memory difficulties. Symptoms experienced in the previous 24 hours are rated on an 11-point scale, from 0 (no symptom) to 10 (worst symptom). Patient Global Impression of Improvement (PGI-I) scale was completed at week 13 (end of treatment).

Acromegaly symptom scores and global impressions of symptom improvement were observed to be maintained or improved with paltusotine treatment. The final version of the Acromegaly Symptom Diary (ASD) was completed by 20 patients in the study. Out of a maximally symptomatic score of 70, median baseline total ASD scores were low [median (IQR)=12.00 (4.21, 23.93) n=20], suggesting low symptom burden at baseline in this study population. Scores showed a slight reduction from Baseline to week 13/EoT [median (IQR)=−0.43 (−5.0, 2.4) n=18. After paltusotine washout there were numeric increases in total ASD scores compared to EoT, however the changes on treatment and during washout were not statistically significant.

Responses to the PGI-I indicated at week 13/EoT, 11 (23.4%) patients had “very much improved” or “much improved”, whereas 26 (55.3%) patients had “minimally improved” or reported “no change” compared to before starting the study, while treated with depot SRLs. No patients reported any degree of worsening.

### Example 8: A Phase 2, Study to Evaluate the Safety and Efficacy of Compound a for the Treatment of Acromegaly (ACROBAT Evolve)

A Phase 2 double-blind, placebo-controlled, randomized withdrawal study designed to evaluate the safety, efficacy, and pharmacokinetics of Compound A in subjects with acromegaly that are responders to octreotide LAR or lanreotide depot.

### Study Design:

Allocation: Randomized. Intervention Model: Parallel Assignment. Masking: Triple (Participant, Care Provider, Investigator). Primary Purpose: Treatment.

### Test Product, Dose, and Mode of Administration:

10 mg HMG capsule formulation. Multiple capsules were swallowed with water depending on dose specified for a given period/cohort.

### Outcome Measures

Primary Outcome Measures: Proportion of subjects who meet responder criteria (based on the mean of two consecutive insulin-like growth factor-1 [IGF-1] measurements ≤upper limit of normal [ULN]) (Time Frame: 13 Weeks).

Secondary Outcome Measures: 1) Change in IGF-1 levels [Time Frame: From Week 10 to Week 13]. 2) Change in growth hormone (GH) levels [Time Frame: From Week 8 to Week 13]. 3) Change in patient assessed symptoms of acromegaly [Time Frame: From Week 10 to Week 13]. Total score computed by adding each of the individual acromegaly symptom intensities (headache pain, joint pain, sweating, fatigue, weakness in legs, swelling, numbness or tingling).

### Eligibility Criteria

### Inclusion Criteria

Male and female subjects 18 to 70 years of age. Confirmed diagnosis of acromegaly that is controlled on stable doses of octreotide LAR or lanreotide depot. Females must be non-pregnant and non-lactating, and either surgically sterile, post-menopausal, or using effective method(s) of birth control. Willing to provide signed informed consent.

### Exclusion Criteria

Treatment naïve acromegaly subjects. Prior treatment with Compound A. Pituitary surgery within 6 months prior to Screening or radiation therapy at any time prior to the study entry. Pituitary radiation therapy (within 3 to 4 years or more than 4 years prior to study entry) with recently documented elevated IGF-1 may be eligible. History or presence of malignancy except adequately treated basal cell and squamous cell carcinomas of the skin within the past 5 years. Use of any investigational drug within the past 30 days or 5 half-lives, whichever is longer. Positive test at Screening for HIV, hepatitis B surface antigen (HBsAg) or hepatitis C antibody (HCV-Ab) or has a history of a positive result. History of alcohol or substance abuse in the past 12 months. Any condition that in the opinion of the investigator would jeopardize the subject's appropriate participation in this study. Cardiovascular conditions or medications associated with prolonged QT or those which predispose subjects to heart rhythm abnormalities. Subjects with symptomatic cholelithiasis. Subjects with clinically significant abnormal findings during the Screening Period, and any other medical condition(s) or laboratory findings that, in the opinion of the Investigator, might jeopardize the subject's safety or ability to complete the study. Subjects who have been taking the following prior medications: pegvisomant (within the last 3 months), dopamine agonists (within the last 3 months) and pasireotide LAR (within the last 6 months). Subjects taking octreotide LAR at a dose higher than 40 mg or lanreotide depot at a dose higher than 120 mg. Subjects who usually take octreotide LAR or lanreotide depot less frequently than every 4 weeks (e.g., every 6 weeks or 8 weeks).

Results from the Phase 2 Trials

25 patients were enrolled: patients treated with octreotide or lanreotide & baseline IGF-1 (×ULN): >1 and <2.5.

Prespecified Primary Analysis Population: patients treated with SRL (octreotide or lanreotide) with elevated IGF-1 at baseline—representing the majority of patients in clinical practice. The primary hypothesis was that the group would show no change in the median IGF-1 at Week 13 versus baseline.

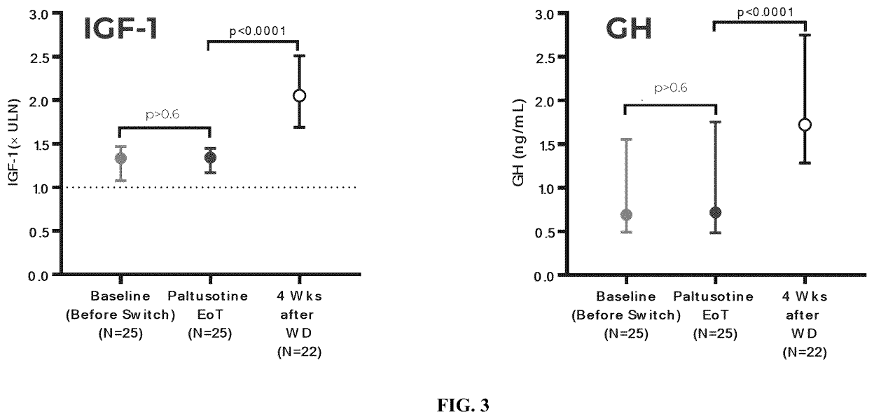

As shown in FIG. 3, paltusotine maintained IGF-1 and GH levels after switching from injected SRL peptide depots (data presented are median (Interquartile Range [IQR]: 25th percentile, 75th percentile) EoT=End of Treatment defined as Week 13 (Visit 14) or last on treatment value carried forward (LOCF). Wks after WD is defined as Week 17 or result at least 22 days after last dose. Note: p-values are based on non-parametric Wilcoxon Sign Rank test of whether the median change is different from zero). IGF-1 levels after 13 weeks of paltusotine treatment did not significantly change from baseline in patients previously treated with injected SRL depots. Rise in IGF-1 after withdrawal (within 2 weeks) which characterized the magnitude of therapeutic activity for oral paltusotine. GH levels after 13 weeks of paltusotine treatment did not significantly change from baseline levels when patients were previously treated with injected SRL depots. Rise in GH after withdrawal characterized the magnitude of therapeutic activity of oral paltusotine.

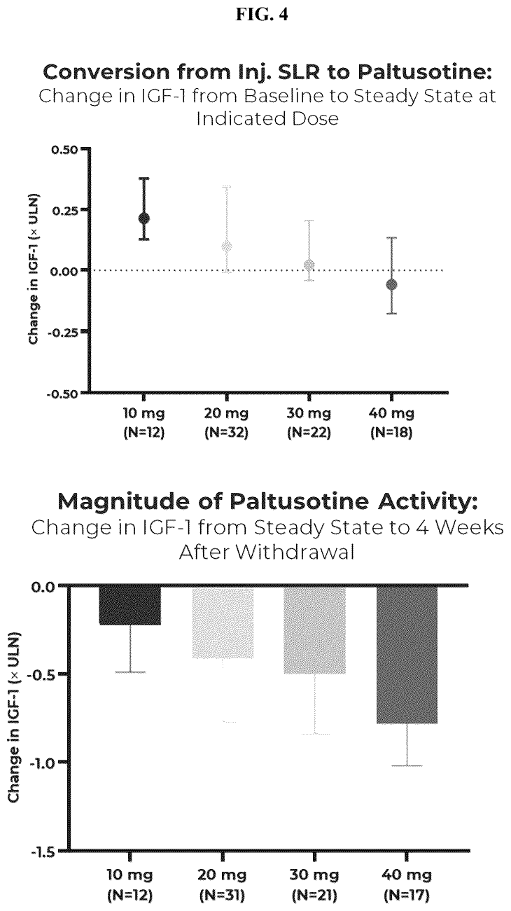

FIG. 4 illustrates the evidence of a dose response observed from the Acrobat Edge and Evolve trials. However, the HMG capsule formulations that were used lacked dose proportional pharmacokinetics above 40 mg.

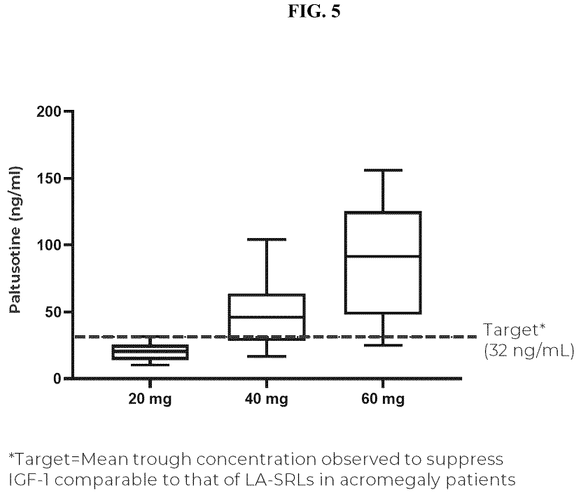

As shown in FIG. 5, administration of the SDD tablets, 40 mg/day and 60 mg/day, is projected to yield trough concentrations consistently in therapeutic range.

In some embodiments, a trough concentration of paltusotine required to provide therapeutic effect comparable to that of long acting SRLs in acromegaly patients is greater than 20 ng/mL, greater than 25 ng/mL, greater than 30 ng/mL, greater than 35 ng/mL, or greater than 40 ng/mL. In some embodiments, a trough concentration of paltusotine required to provide therapeutic effect comparable to that of long acting SRLs in acromegaly patients is at least about 20 ng/mL, at least about 25 ng/mL, at least about 30 ng/mL, at least about 35 ng/mL, at least about 40 ng/mL, or at least about 50 ng/mL. In some embodiments, paltusotine is administered at doses sufficient to provide a trough concentration of paltusotine that is at least about 20 ng/mL, at least about 21 ng/mL, at least about 22 ng/mL, at least about 23 ng/mL, at least about 24 ng/mL, at least about 25 ng/mL, at least about 26 ng/mL, at least about 27 ng/mL, at least about 28 ng/mL, at least about 29 ng/mL, at least about 30 ng/mL, at least about 31 ng/mL, at least about 32 ng/mL, at least about 33 ng/mL, at least about 34 ng/mL, at least about 35 ng/mL, at least about 36 ng/mL, at least about 37 ng/mL, at least about 38 ng/mL, at least about 39 ng/mL, or at least about 40 ng/mL. In some embodiments, paltusotine is administered at doses sufficient to provide a trough concentration of paltusotine that is at least about 32 ng/mL.

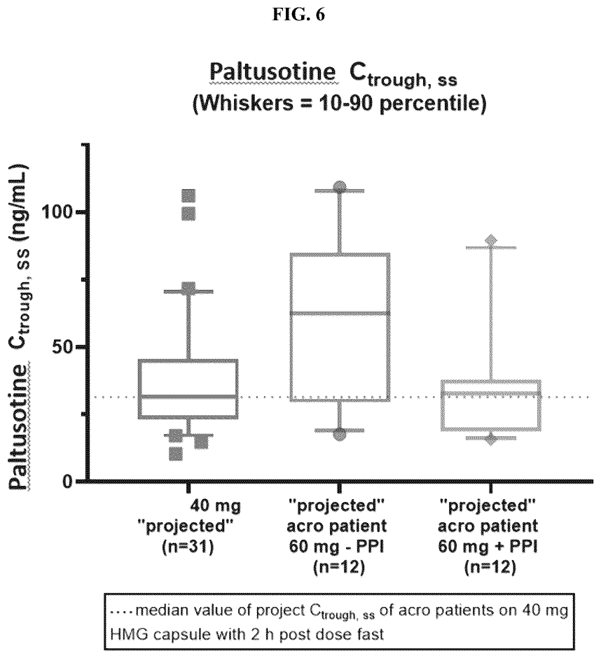

As shown in FIG. 6, the median “projected” steady state trough concentrations of acromegaly patients taking PPI on 60 mg SDD (1 h post dose fast) is similar to the median steady state trough concentrations of acromegaly patients on 40 mg HMG (2 h post dose fast).

### Example 9: Long-Term Study to Evaluate the Safety and Efficacy of Compound A-HCl (Paltusotine) for the Treatment of Acromegaly (ACROBAT Advance)

ACROBAT Advance is an ongoing open-label, long-term (5 year) safety and efficacy study of paltusotine in patients rolling-over from the ACROBAT Evolve & Edge studies.

Eligible patients completing ACROBAT Edge and Evolve entered ACROBAT Advance either immediately on completion of a washout period in the parent study or after a gap during which they reverted to their routine standard of care treatment. Paltusotine therapy: initiated at 10 mg/day and titrated up to maximum dose of 40 mg/day based on IGF-1 and tolerability. Patients were administered 10 mg HMG capsules. Combination therapy was allowed for patients not reaching therapeutic targets with 40 mg/day of paltusotine monotherapy.

IGF-1 measured centrally with IDS-ISYS assay calibrated to WHO recombinant reference standard 02/254.

Baseline characteristics of ACROBAT Advance patients: 41 patients were enrolled. For these patients, it was 129.7 (79.8) months since diagnosis (Mean (SD)). The mean (SD) age was 53.2 (11.5). 23 of the patients were females. 35 of the patients had prior pituitary surgery.

Pre-trial medical treatment (pre-trial is defined as prior to parent trial for direct rollovers and prior to ACROBAT Advance for delayed rollovers) included:

| Pre-trial medical treatment | Number of patients (n) |
|---|---|
| Lanreotide, n - 60/90/120 mg/month | 1/2/13 |
| Octreotide, n - 20/30/40 mg/month | 3/16/3 |
| Pasireotide, n - 40/60 mg/month | 1/1 |
| SRL + Cabergoline, n | 10 |
| Pegvisomant, n - 20 mg/week | 1 |

Cabergoline as administered at a frequency of about 0.5 mg/week to about 0.5 mg/day.

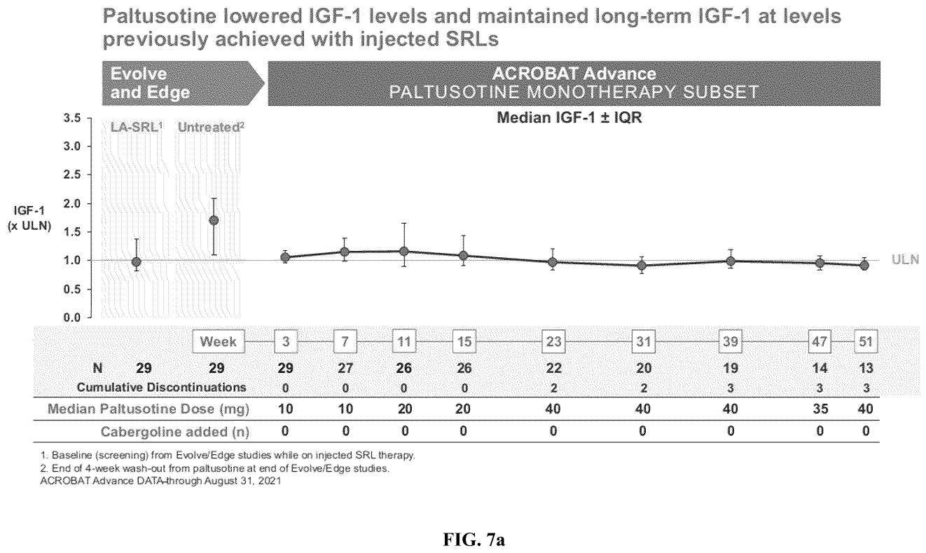

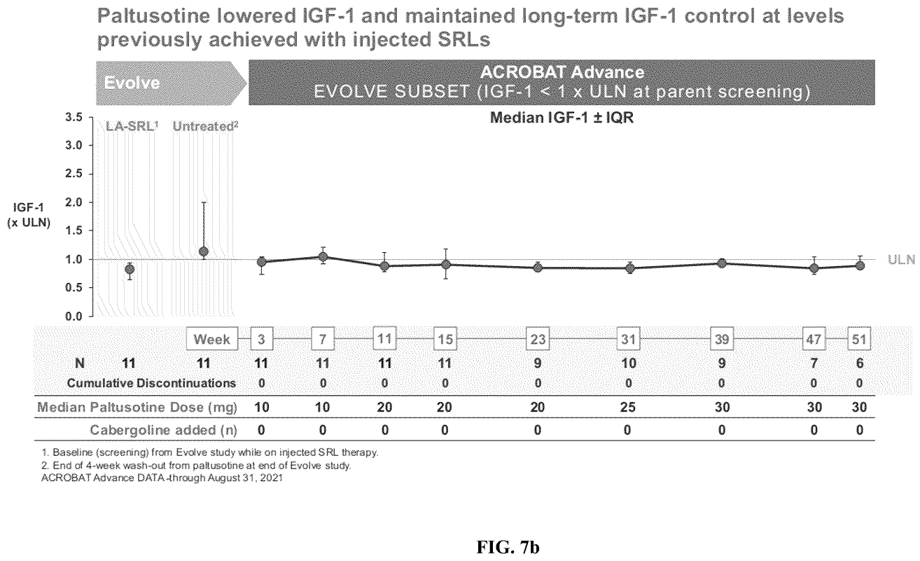

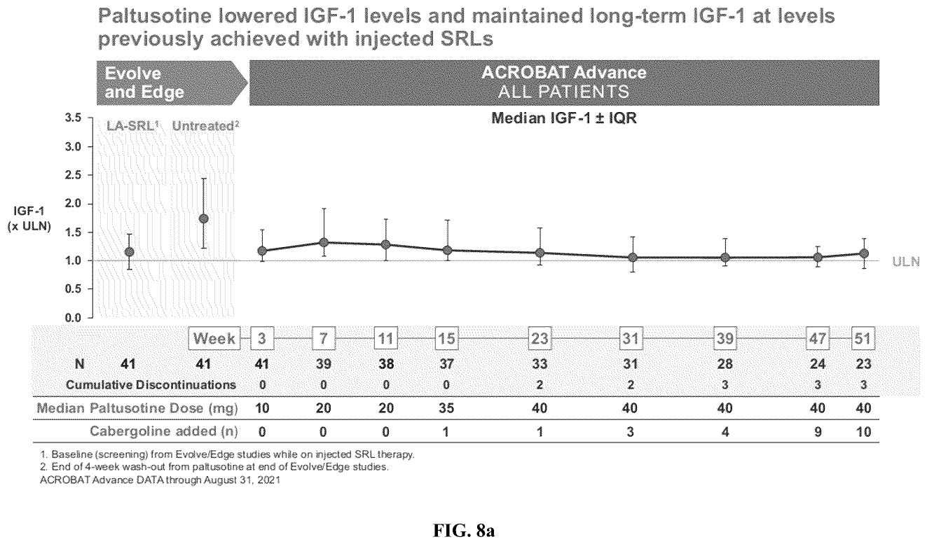

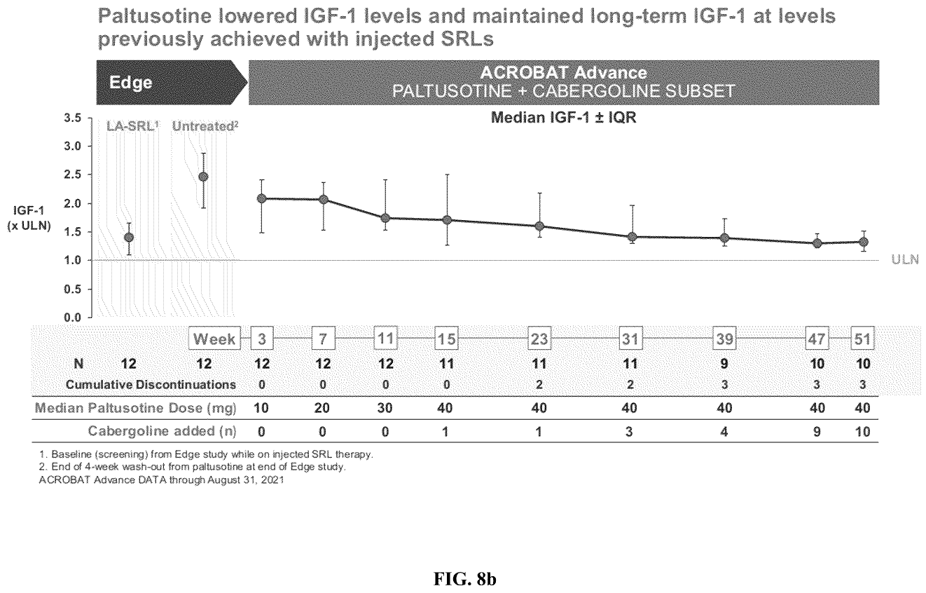

Results from the Phase 2 Trials are shown in FIGS. 7a, 7b, 8a, and 8b.

Once daily, oral paltusotine lowers and maintains IGF-1 at levels comparable to prior injected SRL therapy for up to 51 weeks

Paltusotine is well tolerated with a safety profile similar to that of SRLs, including when used in combination with cabergoline.

### Example 10: A Phase 3 Study to Evaluate the Safety and Efficacy of Compound a for the Treatment of Acromegaly in Subjects with Acromegaly Treated with Long-Acting Somatostatin Receptor Ligands

This is a randomized, controlled, multi-center study, to evaluate the safety and efficacy of Compound A (also known as paltusotine) in subjects with acromegaly treated with long-acting somatostatin receptor ligands

Subjects will be switched from a stable regimen of octreotide/lanreotide to 40 mg paltusotine and their dose titrated up to 60 mg based on biochemical status and tolerability. Down-titration will be allowed based on tolerability. It is expected that by the end of titration period (Week 24) the concentrations of residual pre-trial octreotide or lanreotide will be too low to contribute to efficacy.

### Overall Design

The study includes a Screening Period of 15 to 19 weeks. At the beginning of the Screening Period subjects will receive sponsor-provided long-acting octreotide or lanreotide. At the end of the Screening Period, if subjects are eligible, they will be randomly assigned in a 1:1 ratio to receive either paltusotine or placebo. At the end of the Treatment Period, subjects, who in the opinion of the Investigator, may benefit from treatment with paltusotine, may be enrolled in a long-term open-label extension (OLE) for up to 96 weeks. During the OLE, all subjects will receive paltusotine.

### Inclusion Criteria

Adults ≥18 years of age with medically stable, confirmed-active acromegaly and on an approved, stable dose of long-acting octreotide or lanreotide, for at least 12 weeks prior to Screening. Continuous treatment with octreotide or lanreotide monotherapy must be at least 12 weeks prior to Screening. The following prior treatment regimens are allowed for inclusion into this study: long-acting octreotide: 10, 20, 30, 40 mg every 4 weeks or long-acting lanreotide: 60, 90, 120 mg every 4 weeks or 120 mg every 6 or 8 weeks.

Previous diagnosis of acromegaly. This requires evaluable documentation of a pituitary tumor diagnosed by pituitary imaging or histopathologic confirmation of a pituitary adenoma at least 24 weeks prior to Screening.

For subjects who have had pituitary surgery, there must be documentation of IGF-1 concentration ≥1.3×ULN at least 12 weeks after last pituitary surgery. The surgery must have been performed ≥24 weeks prior to Screening. Subjects who have not had pituitary surgery must have documentation of IGF-1 concentration ≥1.3×ULN performed ≥24 weeks prior to Screening.

### Screening and Testing Evaluations

IGF-1 must be ≤1.2×ULN for the subject to start treatment with sponsor-provided medication. At the end of the Screening Period (Weeks −8 and −4), subjects will undergo 2 IGF-1 tests to reconfirm eligibility. The average of the 2 measurements must meet the IGF-1 criteria for eligibility. If a third IGF-1 measurement is needed (Week −2), the average of the 3 measurements must meet the IGF-1 criteria for eligibility.

### Exclusion Criteria

Treatment-naïve or treatment-withdrawn acromegaly subjects. History of pituitary radiation therapy. Subjects with adrenal insufficiency who are not receiving adequate adrenal replacement therapy at the time of Screening.

High risk pituitary tumor pattern as defined by: compression of the optic chiasm or invasion of adjacent brain structures (other than sphenoid sinus or cavernous sinus); history of tumor growth within 1 year after surgery (unless it occurred during a period of medical therapy interruption); anticipated requirement for neurosurgical intervention or radiation therapy within the time course of the study; pituitary carcinoma currently or at any time in the past.

History of major surgery/surgical therapy for any cause within 4 weeks prior to Screening.

Active malignant disease within the last 5 years.

Use of the following medications: pasireotide LAR (within 24 weeks prior to Screening), pegvisomant (within 12 weeks before Screening), dopamine agonists (within 12 weeks before Screening), or short acting somatostatin analogs (SA-SSAs) within last 12 weeks before the first dose of study drug. Withdrawal of these medications should be part of the subject's medical care plan prior to Screening.

Current or recent use of medications that would interefere with the study.

### Screening

The study includes a Screening Period of 15 to 19 weeks. IGF-1 must be ≤1.2×ULN for the subject to start treatment with sponsor-provided long-acting octreotide or lanreotide. The dosing interval of lanreotide/octreotide injections used prior to the study (every 4 weeks, every 6 weeks, or every 8 weeks) should be maintained during the Screening period. This administration will be done in the investigator site. If IGF-1 is not ≤1.2×ULN the subject is considered a screening failure.

Towards the end of the Screening Period (Weeks −8 and −4), subjects will undergo 2 IGF-1 tests to reconfirm eligibility. The average of the 2 measurements must meet the IGF-1 criteria for eligibility. If a third IGF-1 measurement is needed (Week −2), the average of the 3 measurements must meet the IGF-1 criteria for eligibility.

Dosing intervals for sponsor-provided octreotide/lanreotide are presented below.

### Sponsor-Provided Lanreotide/Octreotide Dosing During Screening

Sponsor-provided Lanreotide/Octreotide Dosing During Screening

| Lanreotide/Octreotide | |
|---|---|
| Dosing Interval | Injection Timing |
| Every 4 weeks | 3 injections: first injection Week −12, second injection Week −8, |
| | third injection Week −4 |
| | The interval between the third injection and Day 1 = 4 weeks ± 3 days |
| Every 6 weeks | 2 injections: first injection Week −12, second injection Week - 6 |
| | The interval between the second injection and Day 1 = 6 weeks ± 3 days |
| Every 8 weeks | 2 injections: first injection Week −16, second injection Week −8 |
| | The interval between the second injection and Day 1 = 8 weeks ± 3 days |

### Treatment Period (Randomized, Controlled Phase [RC Phase])

Subjects meeting all eligibility criteria and an average Screening Period IGF-1 ≤1.0×ULN will be randomized (1:1) to either paltusotine (n=26) or placebo (n=26).

The octreotide/lanreotide injection is the key temporal event. Day 1 (start of treatment) must be 4 weeks (±3 days) or 6 weeks (+3 days) after the last dose of lanreotide if a subject is administered his/her octreotide/lanreotide every 4 weeks or 6 weeks, respectively. Additionally, if a subject is administering his/her lanreotide every 8 weeks, Day 1 (start of treatment) must be 8 weeks (+3 days) after the last dose of lanreotide.

The Treatment Period will be approximately 36 weeks with 11 planned visits.

Subjects will not be administered sponsor-provided octreotide/lanreotide on Day 1. Instead, subjects will receive their first dose of study drug on site.

If rescue therapy is required during the RC phase, subjects will discontinue paltusotine or placebo, initiate rescue therapy, and continue the study for the remainder of the 36-week RC phase.

### Open Label Extension Phase

Subjects who complete the 36-week RC phase can continue participation in the OLE phase if the subject is willing to participate and the subject would benefit from continued participation and treatment with paltusotine. The OLE Treatment Period is 96 weeks with 11 planned visits. Data from the EOR visit of the RC phase will be used as baseline of the OLE, unless otherwise specified.

### Objectives and Endpoints for the Controlled Part of the Study

### Primary Objectives

To evaluate the effect of paltusotine versus placebo on IGF-1 response. Endpoints for this objective include the proportion of subjects who maintain biochemical response in IGF-1 (≤1.0× the upper limit of normal [ULN]) at the End of the Randomized Control Phase (EOR).

### Secondary Objectives

To evaluate the effect of paltusotine versus placebo on IGF-1 level. Endpoints for this objective include the change from baseline in IGF-1, in units of ULN, to EOR.

To evaluate the effect of paltusotine versus placebo on GH response. Endpoints for this objective include the proportion of subjects with GH<1.0 ng/mL at Week 34, out of those who had GH<1.0 ng/mL at Screening.

To evaluate the effect of paltusotine versus placebo on acromegaly symptoms. Endpoints for this objective include the change from baseline in Total Acromegaly Symptoms Diary (ASD) score to EOR.

### Exploratory Objectives

To evaluate the effect of paltusotine versus placebo on GH level. Endpoints for this objective include the change from baseline to EOR in GH.

To evaluate the effect of paltusotine versus placebo on the need for rescue therapy. Endpoints for this objective include the proportion of subjects who receive rescue therapy.

To evaluate the effect of paltusotine versus placebo on biochemical response. Endpoints for this objective include the proportion of subjects who achieve IGF-1≤1.3×ULN at EOR; the proportion of subjects who achieve GH<2.5 ng/mL at Week 34, out of those who had GH<2.5 ng/mL at Screening.

### Safety Objectives

To evaluate the safety and tolerability of paltusotine versus placebo in subjects with acromegaly. Endpoints for this objective include: the incidence of treatment-emergent adverse events (TEAEs), including serious adverse events (SAEs) and TEAEs that lead to discontinuation; change in safety parameters; incidence of clinically significant changes in abdominal (gallbladder) ultrasound compared with baseline; change from baseline in residual pituitary tumor volume.

### Endpoints for the Open-Label Extension

Safety endpoints: incidence of TEAEs, including serious adverse events (SAEs) and TEAEs leading to discontinuation of study drug; change in quantitative safety parameters; change from baseline in residual pituitary tumor volume.

Additional efficacy endpoints: change from baseline in IGF-1 and GH levels; proportion of subjects with IGF-1≤1.0×ULN; proportion of subjects with IGF-1≤1.3×ULN; proportion of subjects who receive permitted adjunctive standard acromegaly treatment.

### Number of Subjects

The total sample size is 52 subjects (26 in the paltusotine arm, 26 in the placebo arm).

### Duration and Intervention Groups

The study comprises 2 phases: an RC phase and long-term OLE phase. Subjects who participate in both phases will complete approximately 144 weeks (almost 3 years) of treatment and observation. The study will consist of:
Screening Period: 15 or 19 weeks; Week −19/−15 through Day −1
RC phase (36 weeks): Treatment Period: Day 1 through Week 36; the end of Week 36 is defined as EOR. Week 1 through Week 24 will be the dose Titration Period.
OLE (96 weeks): Eligible subjects who complete the RC phase may enter the OLE and continue or initiate treatment with paltusotine until end of treatment (EOT); the end of 96 weeks of the OLE phase is the planned EOT.

### Study Drug

During the RC and OLE phases, paltusotine will be provided as 20 mg tablets. Matching placebo tablets will be identical in appearance. The starting dose will be 40 mg (2×20 mg tablets of active paltusotine or matching placebo) once daily for oral self-administration.

### Dose Adjustment During the RC Phase

The dose of study drug can be titrated up to 60 mg once daily (QD). Dose levels may increase from 40 mg (2×20 mg tablets) to 60 mg (3×20 mg tablets) based on IGF-1 response. A fixed or stable dose is considered to be established when IGF-1 level is ≤0.9×ULN, confirmed by 2 successive IGF-1 measurements collected approximately 4 weeks apart.

Dose up-titration will be based on 2 criteria:
Acceptable tolerability at the current dose, as evaluated by the Investigator, and
The subject's most recent IGF-1 result is >0.9×ULN

| Dose | | |
|---|---|---|
| Titration/Adjustment | IGF-1 Criteria | Tolerability Criteria |
| 40 mg to 60 mg | Subject’s most recent IGF-1 | Increase allowed due to acceptable |
| | results on 40 mg is >0.9 × ULN | tolerability on 40 mg |
| 40 mg to 20 mg | | Dose reduction required due to |
| | | unacceptable tolerability on 40 mg |
| 20 mg to 40 mg | Subject’s most recent IGF-1 | Tolerability is acceptable on 20 mg |
| | results on 20 mg is >0.9 × ULN | |
| 60 mg to 40 mg | | Dose reduction required due to |
| | | unacceptable tolerability on 60 mg |

A subject's dose may be reduced during the RC phase due to poor tolerability. In general, a TEAE of severe intensity for which there is a reasonable possibility it is caused by (related to) study drug would be expected to result in study drug dose reduction. If toleration improves following a dose reduction, the dose may increase in 20 mg increments, if needed, based on protocol-specified dose titration criteria to a maximum of 60 mg (3×20 mg tablets). When the study medication is not tolerated, the study medication may be held for up to a total of 14 days per year, but no more than 7 consecutive days per year during the study, followed by resumption of the study medication at the same or reduced dose as appropriate.

### RC Phase Rescue Criteria

During the RC phase, acromegaly treatments other than study drug are prohibited during the study. Criteria for rescue with pre-trial acromegaly medication (injected long-acting octreotide or lanreotide) consist of the following: 1) significant worsening of 1 or more acromegaly symptoms or the development of a new acromegaly symptom, at the highest dose (60 mg) for at least 2 weeks, (“significant worsening” may be defined as symptoms requiring a substantial increase in level of clinical care or substantial clinical deterioration; and 2) IGF-1 value ≥1.3×ULN at the highest dose (60 mg) measured at 2 successive visits.

### Dosage and Dose Adjustment During the Open-Label Extension

The starting dose regimen for all subjects who participate in the OLE will be paltusotine 40 mg QD. Dose escalation in the OLE will generally occur once the first IGF-1 result is available. At that time, the Investigator can determine whether dose can be up-titrated to 60 mg.

### Open-Label Extension Adjunctive Treatments

During the OLE, non-SRL acromegaly treatments, such as oral dopamine agonists (e.g., cabergoline) or GH receptor antagonist pegvisomant are permitted and may be initiated as an adjunct when paltusotine is at the highest tolerated dose for at least 4 weeks and IGF-1 is persistently ≥1.3×ULN or otherwise not at the subject's therapeutic target in the judgement of the Investigator.

If it is determined that adjunctive medication is required, then use of a dopamine agonist (e.g., cabergoline) would be the first-line adjunct. If after an adequate period of assessment, study medication combined with a dopamine agonist does not achieve therapeutic targets, then pegvisomant may be added as a second-line adjunct. Pegvisomant may be added to the paltusotine+dopamine agonist drug combination. If there was no evidence of a therapeutic response to combined paltusotine+dopamine agonist, the dopamine agonist should be stopped and pegvisomant added to paltusotine therapy

### Example 11: A Phase 3, Study to Evaluate the Safety and Efficacy of Compound a for the Treatment of Acromegaly in Subjects with Non-Pharmacologically Treated Acromegaly

This is a randomized, controlled, multicenter study to evaluate the safety and efficacy of Compound A (also known as paltusotine) in subjects with non-pharmacologically treated acromegaly.

### Overall Design

This is a Phase 3, multicenter, randomized, double-blind, placebo-controlled study where subjects with non-pharmacologically treated acromegaly will be randomly allocated to receive either paltusotine or placebo. A total of 70 subjects will targeted for enrollment.

The Screening Period for this study may be approximately 4, 8, or 12-16 weeks, depending on prior treatment. After the Screening Period, subjects will be enrolled in a 12-week Randomized Controlled (RC) phase and randomly assigned in a 1:1 ratio to receive Compound A or matching placebo stratified by prior treatment (medically naïve or previously treated versus washout). At the end of the RC phase (EOR), subjects may be enrolled in a long-term open-label extension (OLE) study, during which they will receive paltusotine for up to 96 weeks.

### Inclusion Criteria

Subjects ≥18 years of age at the time of Screening, who have had at least 1 pituitary surgery but have persistent active acromegaly and who fall into 1 of the following 3 groups are eligible to participate in the study.
Medically naïve group: Those who have not been previously treated with acromegaly medications (including long-acting somatostatin receptor ligands [LA-SRLs]) who at Screening have IGF-1>1.1× the upper limit of normal (ULN).
Previously Treated group (no treatment within previous 4 months): Subjects who have last been treated with acromegaly medications at least 4 months prior to Screening and who have IGF-1>1.1×ULN at Screening.
Washout group: Subjects at Screening who are receiving stable treatment (no change in dose for 3 months prior to Screening) with octreotide monotherapy, who have IGF-1≤1.0×ULN at Screening Visit 1, and are willing to washout of their medication during the Screening Period. Any form of pre-trial octreotide monotherapy (long- or short-acting octreotide [subcutaneous (SC) or oral]) can be washed out and will determine the duration of the Screening Period. After informed consent is provided, the subject should not receive further pre-trial acromegaly medication. IGF-1 must rise at least 30% from the first Screening Visit to the last Screening Visit and to >1.1×ULN to qualify for enrollment.

Previous diagnosis of acromegaly. This requires evaluable documentation of a pituitary adenoma at least 24 weeks prior to Screening. In addition, there must be documentation of IGF-1 concentration >1.1×ULN at least 12 weeks after last pituitary surgery.

### Exclusion Criteria

History of ineffectiveness or significant intolerance of octreotide or lanreotide treatment.

History of pituitary radiation therapy within 3 years of Screening. Subjects with adrenal insufficiency who are not receiving adequate adrenal replacement therapy at the time of Screening, as determined by the Investigator. High risk pituitary tumor pattern as defined by: compression of the optic chiasm or invasion of adjacent brain structures; history of tumor growth within 1 year after surgery or radiation; anticipated requirement for neurosurgical intervention or radiation therapy within the time course of the study. Pituitary carcinoma currently or at any time in the past.

History of major surgery/surgical therapy for any cause within 4 weeks of Screening.

Active malignant disease within the last 5 years.

Use of the following medications: Lanreotide (within 16 weeks before Screening), pasireotide LAR (within 24 weeks prior to Screening), pegvisomant (within 16 weeks before Screening), dopamine agonists (within 16 weeks before Screening), any combination of 2 or more acromegaly medications at Screening, proton pump inhibitors (from start of Screening) until the end of the study. Withdrawal of these medications should be part of the subject's medical care plan prior to Screening.

Current or recent use of medications that would interefere with the study.

### Treatment Period (Randomized, Controlled (RC) Phase)

Once all screening assessments are completed, the subject will be randomized to treatment on Day 1 of the Treatment Period. The Treatment Period will be approximately 12 weeks. Subjects will receive their first dose of study drug on Day 1 (paltusotine or placebo).

If rescue is required during the RC phase, subjects will discontinue paltusotine or placebo, and will be considered non-responders. They will undergo the Early Termination (ET) visit, and will be offered open-label extension (OLE) participation. If the subject does not participate in the OLE, the subject will return to standard acromegaly care.

Open-label Extension (approximately 100 weeks, including a 4-week follow-up)

Subjects who complete the 12-week RC phase or who meet rescue criteria can continue participation in the OLE phase if the subject may benefit from continued participation and treatment with paltusotine and the subject is willing to participate. The OLE Treatment Period is 96 weeks.

### Study Drug

Paltusotine will be provided as 20 mg tablets. Matching placebo tablets will be identical in appearance to paltusotine tablets. The starting daily dose will be 20 mg (1×20 mg tablet of active paltusotine or matching placebo) for oral self-administration.

Study drug will be swallowed in the morning, with at least 8 ounces (237 mL) of water, after an overnight fast of at least 6 hours. No food or drink (except for water), or another medication will be allowed for at least 1 hour after drug administration.

Special precaution for antacid treatment: famotidine cannot be taken later than dinner time. Other H2 blockers and antacids can be taken at bedtime. The purpose being to minimize the increase in gastric pH the next morning because this may decrease absorption of paltusotine.

The last dose of study drug for the RC phase will be self-administered the day prior to the Week 12/EOR visit. The first dose of the OLE phase will be administered during the Week 12/EOR visit for those continuing into the OLE.

### Dose Titration and Adjustment

Beginning with the Week 6 visit and prior to the Week 11 visit, dose up-titration will be based on 2 criteria:
Acceptable tolerability at the current dose, as evaluated by the Investigator, and
The subject's most recent IGF-1 result is >0.9×ULN

| DoseTitration/ | | |
|---|---|---|
| Adjustment | IGF-1 Criteria | Tolerability Criteria |
| 20 mg to 40 mg | | Increase required after 1 week of dosing |
| | | (Day 8) if acceptable tolerability on 20 mg |
| 40 mg to 60 mg | Week 4 IGF-1 > 0.9 × ULN or at | Acceptable tolerability on 40 mg |
| | any time prior to Week 11 visit | |
| 60 mg to 40 mg | | Dose reduction required due to |
| | | unacceptable tolerability on 60 mg |
| 40 mg to 20 mg | | Dose reduction required due to |
| | | unacceptable tolerability on 40 mg |

If rescue treatment is required for worsening acromegaly during the RC phase, subjects will discontinue paltusotine or placebo, and either enter the OLE or discontinue from the study. Rescue criteria are as follows:
Significant worsening of 1 or more acromegaly symptoms or the development of a new acromegaly symptom, at the highest dose (60 mg) for at least 2 weeks (“significant worsening” may be defined as symptoms requiring a substantial increase in level of clinical care or substantial clinical deterioration); and
IGF-1 value increased from baseline by >30% at the highest dose (60 mg) measured twice, at a planned visit or at an unscheduled visit if an earlier result is needed; and

If rescue is required during the RC phase, subjects will discontinue paltusotine or placebo, and will be considered non-responders.

### Dosage and Dose Adjustment During the Open-Label Extension

The starting daily dose regimen for all subjects who participate in the OLE will be paltusotine 20 mg (1×20 mg tablet). Subjects will be instructed to take 1 tablet per day for the first week and then if tolerated, increase to 40 mg QD (2×20 mg tablets) until the next study visit.

Further up-titration (e.g., to 60 mg QD (3×20 mg tablets)) in the OLE may occur once the first IGF-1 result is available.

### Open-Label Extension Adjunctive Treatments

During the OLE, non-SRL acromegaly treatments, such as oral dopamine agonists (e.g., cabergoline) or GH receptor antagonist (i.e., pegvisomant) are permitted and may be initiated as an adjunct when paltusotine is at the highest tolerated dose for at least 4 weeks and IGF-1 is persistently ≥1.3×ULN, or otherwise not at the subject's therapeutic target.

If it is determined that adjunctive medication is required, then use of a dopamine agonist (e.g., cabergoline) would be the first-line adjunct. If after an adequate period of assessment, study medication combined with a dopamine agonist does not achieve therapeutic targets, then pegvisomant may be added as a second-line adjunct. Pegvisomant may be added to the paltusotine+dopamine agonist drug combination. If there was no evidence of a therapeutic response to combined paltusotine+dopamine agonist, the dopamine agonist should be stopped and pegvisomant added to paltusotine therapy.

### Prohibited Medicine

Prohibited medications include: proton pump inhibitors; use of famotidine in the evenings; oral estrogen, except for monophasic estrogen-progestin oral contraception or stable estrogen replacement; strong inducers of the drug metabolizing enzyme CYP3A4; any standard acromegaly drug during the RC phase of the study; octreotide (any form), lanreotide, or pasireotide during the OLE; any other investigational drug.

### Primary Objectives

To evaluate the effect of paltusotine versus placebo on IGF-1 response. Endpoints for this objective include the proportion of subjects who achieve biochemical response in IGF-1 (1.0×ULN) at the End of the Randomized Controlled phase (EOR).

### Secondary Objectives

To evaluate the effect of paltusotine versus placebo on IGF-1 levels. Endpoints for this objective include the change from baseline in IGF-1, in units of ULN, to EOR.

To evaluate biochemical response by various definitions following treatment with paltusotine. Endpoints for this objective include the proportion of subjects who achieve IGF-1 ≤1.3×ULN at EOR; proportion of subjects with GH<2.5 ng/mL at EOR; proportion of subjects with GH<1 ng/mL at EOR.

To evaluate the effect of paltusotine versus placebo on symptoms of acromegaly. Endpoints for this objective include the change from baseline to EOR in Acromegaly Symptoms Diary (ASD) total scores.

### Exploratory Objectives

To evaluate the effect of paltusotine versus placebo on GH level. Endpoints for this objective include the change from baseline to EOR in GH.

To evaluate the effect of paltusotine versus placebo on the need for rescue treatment. Endpoints for this objective include the proportion of subjects who need rescue treatment from start of treatment through EOR.

### Safety Objectives

To evaluate the safety and tolerability of paltusotine versus placebo. Endpoints for this objective include: the incidence of treatment-emergent adverse events (TEAEs), including serious TEAEs and TEAEs leading to discontinuation; change from baseline in safety parameters; incidence of clinically significant changes in abdominal (gallbladder) ultrasound compared with baseline

### Endpoints for the Open-Label Extension (OLE)

Safety endpoints: Incidence of TEAEs, including serious adverse events (SAEs), and TEAEs leading to discontinuation of study drug; change in safety parameters; change from baseline in residual pituitary tumor volume.

Additional efficacy endpoints: change from baseline in IGF-1 and GH levels; Proportion of subjects with IGF-1≤1.0×ULN; proportion of subjects who discontinue RC (through rescue) and enter OLE, that then achieve IGF-1≤1.0×ULN during OLE; proportion of subjects who receive permitted adjunctive standard acromegaly treatment.

### Subject-Reported Assessments

Subject-reported assessments include: (1) Acromegaly Symptoms Diary (ASD), (2) Acromegaly Quality of Life (AcroQoL), (3) EQ-5D-5L, (4) Patient Global Impression of Severity (PGI-S), (5) Patient Global Impression of Improvement (PGI-I), and (6) Treatment Preference Questionnaire. These assessments will be completed using an electronic device (either through an application on the subject's electronic device or by a device provided by the Sponsor).

### Acromegaly Symptoms Diary

Subjects will be asked to complete the ASD, a brief symptom diary, daily at home beginning approximately 2 weeks prior to study drug dosing for the duration of the randomized controlled phase. The ASD should be completed at approximately the same time of day, as consistently as possible.

The ASD consists of 9 items (headache pain; joint pain; sweating; fatigue; weakness in legs; swelling; numbness or tingling; difficulty sleeping; and a question on short-term memory), each ranked in intensity from 0-10. The total ASD score will be computed by adding the individual symptom intensities for headache pain; joint pain; sweating; fatigue; weakness in legs; swelling; and numbness or tingling, therefore total ASD score can range from 0-70. All individual item scores, including difficulty sleeping and the question on short-term memory will be collected and analyzed in individual item scoring.

### Acromegaly Quality of Life Questionnaire

The AcroQoL assesses health-related quality of life in people with acromegaly. The questionnaire consists of 22 questions in 2 domains: physical aspects (8 questions) and psychological aspects (14 questions). The psychological aspects domain is divided into 2 sub-scales addressing physical appearance (7 questions) and the impact of the disease on personal relationships (7 questions). Each of the question is answered on a 1 to 5 scale (always, most of the time, sometimes, rarely, or never). Higher scores indicate a better quality of life.

### EQ-5D-5L

The EQ-5D-5L (5 severity levels EQ-5D), developed by the EuroQoL, is a standardized instrument to be completed by the subject for use as a measure of health outcomes applicable to a wide range of health conditions. It comprises 5 dimensions of health: mobility, ability to self-care, ability to undertake usual activities, pain and discomfort, and anxiety and depression.

Based on qualitative and quantitative studies conducted by the EuroQoL Group, there are 5 options (levels) under each domain: ‘no problems’, ‘slight problems’, ‘moderate problems’, ‘severe problems’ and ‘unable to/extreme problems’. The responses to all 5 dimensions, can be converted to a single summary index, utility (range: 0 to 1), by using value sets. Higher index values represent better health states.

### Global Impressions of Improvement and Severity

The PGI-I is a 7-point scale designed to measure symptomatic change at a specific time as compared with baseline. Treatment response with the PGI-I ranges from a score of 1 “very much improved” to 7 “very much worse”. Lower scores indicate improvement. The PGI-S is also rated on a 7-point Likert scale ranging 1 “normal, not all ill to 7 “among the most extremely ill”. Higher scores indicate more severe disease.

The examples and embodiments described herein are for illustrative purposes only and various modifications or changes suggested to persons skilled in the art are to be included within the spirit and purview of this application and scope of the appended claims.

What is claimed is:

1. A method of treating acromegaly in a human comprising orally administering to the human a pharmaceutical composition comprising 3-[4-(4-amino-piperidin-1-yl)-3-(3,5-difluoro-phenyl)-quinolin-6-yl]-2-hydroxy-benzonitrile (Compound A), or a pharmaceutically acceptable salt thereof; wherein the human was previously treated with, and responded to and tolerated treatment with, a somatostatin analog.

2. The method of claim 1, wherein administration of the pharmaceutical composition comprising Compound A, or a pharmaceutically acceptable salt thereof, to the human maintains IGF-1 levels in the human at levels comparable to prior treatment with the somatostatin analog.

3. The method of claim 1, wherein administration of the pharmaceutical composition comprising Compound A, or a pharmaceutically acceptable salt thereof, to the human maintains a normal serum IGF-1 level in the human.

4. The method of claim 1, wherein administration of the pharmaceutical composition comprising Compound A, or a pharmaceutically acceptable salt thereof, to the human maintains serum IGF-1 at ≤1.0× the upper limit of normal (ULN) in the human.

5. The method of claim 2, wherein administration of the pharmaceutical composition comprising Compound A, or a pharmaceutically acceptable salt thereof, to the human maintains IGF-1 levels in the human for up to 51 weeks.

6. The method of claim 1, wherein an amount of Compound A, or a pharmaceutically acceptable salt thereof, that is equivalent to 40 mg of Compound A is administered to the human once daily.

7. The method of claim 1, wherein an amount of Compound A, or a pharmaceutically acceptable salt thereof, that is equivalent to 60 mg of Compound A is administered to the human once daily.

8. The method of claim 1, wherein an amount of Compound A-monohydrochloride that is equivalent to 40 mg of Compound A is administered to the human once daily.

9. The method of claim 1, wherein an amount of Compound A-monohydrochloride that is equivalent to 60 mg of Compound A is administered to the human once daily.

10. The method of claim 1, wherein the somatostatin analog is octreotide, lanreotide, or pasireotide.
